(19) United States
(12) Patent Application Publication
(10) Pub. No.: US 2010/0167933 A1
(43) Pub. Date: Jul. 1, 2010

(54) Piperazine Compounds With Herbicidal Action

(76) Inventors: Eike Hupe, Mannheim (DE); Thomas Seitz, Viernheim (DE); Matthias Witschel, Bad Durkheim (DE); Dschun Song, Mannheim (DE); William Karl Moberg, Hassloch (DE); Liliana Parra Rapado, Offenburg (ES); Frank Stelzer, Mannheim (DE); Andrea Vescovi, Barcelona (ES); Trevor William Newton, Neustadt (DE); Robert Reinhard, Limburgerhof (DE); Bernd Sievernich, Hassloch (DE); Klaus Grossmann, Neuhofen (DE); Thomas Ehrhardt, Speyer (DE); Michael Rack, Eppelheim (DE); Elmar Kibler, Hassloch (DE)

Correspondence Address: BRINKS, HOFER, GILSON & LIONE, P.O. BOX 1340, MORRISVILLE, NC, 27560, US

(21) Appl. No.: 12/663,756

(22) PCT Filed: Jun. 20, 2008
(86) PCT No.: PCT/EP08/57830

§ 371 Date: Dec. 9, 2009

(30) Foreign Application Priority Data
Jun. 22, 2007 (EP) 07110913.6

(51) Int. Cl. A01N 43/60; C07D 241/04; A01P 13/00

(52) U.S. Cl. 504/235; 544/383

(57) ABSTRACT

The present invention relates to piperazine compounds of the general formula I defined below and to their use as herbicides. Moreover, the invention relates to compositions for crop protection and to a method for controlling unwanted vegetation.

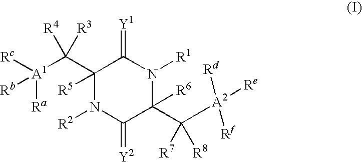

In formula I, A¹, A² independently of one another are aryl or heteroaryl, where R^{a} is attached in the ortho-position to the point of attachment of A¹ to a carbon atom or a nitrogen atom of A¹, Y¹, Y² are oxygen, sulfur or a group NR^{y1}, and the variables R^{a}, R^{b}, R^{c}, R^{d}, R^{e}, R^{f}, R¹, R², R³, R⁴, R⁵, R⁶, R⁷, and R⁸ have the meanings mentioned in the claims and the description.

The present invention relates to piperazine compounds of the general formula I defined below and to their use as herbicides. Moreover, the invention relates to compositions for crop protection and to a method for controlling unwanted vegetation.

The thaxtomins A and B (King R. R. et al., J. Agric. Food Chem. (1992) 40, 834-837), which are produced by the plant pathogen S. scabies, are natural products having a central piperazine-2,5-dione ring which carried a 4-nitroindol-3-ylmethyl radical in the 3-position and an optionally OH-substituted benzyl radical in the 2-position. Because of their plant-damaging activity, this class of compounds was also examined for a possible use as herbicides (King R. R. et al., J. Agric. Food Chem. (2001) 49, 2298-2301).

EP-A 181152 and EP-A 243122 describe piperazine compounds of a similar structure and their use as antagonists of the platelet activating factor.

WO 99/48889, WO 01/53290 and WO 2005/011699 describe 2,5-diketopiperazine compounds having in one of the 3- and 6-positions a 4-imidazolyl radical which is attached via a methylene or methyne group and in the other 3- or 6-position a benzyl or benzylidene radical. These compounds have antitumor activity.

US 2003/0171379 A1 describes the use of mactanamide, a fungistatic diketopiperazine of the formula A

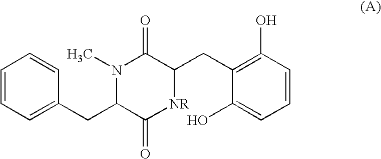

in which R is H or methyl, as an antiinflammatory in medicine.

In the context of synthetic investigations into the preparation of thaxtomin A and B, J. Gelin et al., J. Org. Chem. 58, 1993, pp. 3473-3475, and J. Moyroud et al., Tetrahedron 52, 1996, pp. 8525-8543 describe dehydrothaxtomin derivatives. Described are, inter alia, compounds of the formula

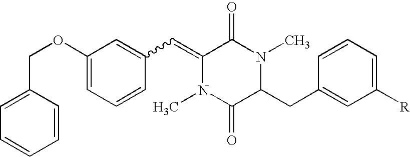

in which R is hydrogen or NO₂.

N. Saito et al., J. Chem. Soc. Perkin Trans 1997, pp. 53-69 describe, inter alia, compounds of the formula below

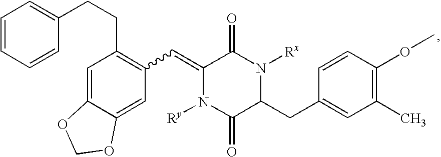

in which R^{y} is hydrogen or benzyl and R^{x} is hydrogen, acetyl or isopropyloxycarbonyl as precursors for the preparation of ecteinascidins.

In the context of synthetic investigations into the preparation of phthalascidin, Z. Z. Liu et al., Chinese Chem. Lett. 13(8) 2002, pp. 701-704 describe an intermediate of the formula below, in which Bn is benzyl:

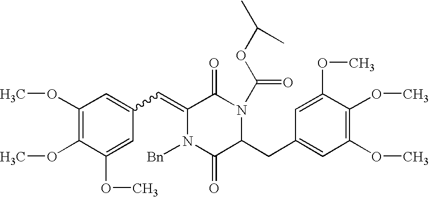

J. Bryans et al., Journal of Antibiotics 49(10), 1996, pp. 1014-1021 describe the compound of the formula below:

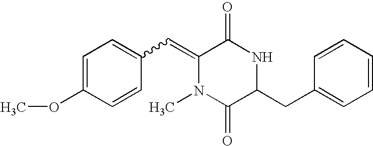

The earlier patent application PCT/EP2007/050067 (=WO 2007/077247) describes 2,5-diketopiperazine compounds which have an aryl or hetaryl radical attached via a methyne group in the 3-position and an aryl or hetaryl radical attached via a methylene group in the 6-position.

The earlier patent application PCT/EP2006/070271 (=WO 2007/077201) describes 2,5-diketopiperazine compounds which have an aryl or hetaryl radical attached via a methylene group both in the 3-position and in the 6-position.

It is an object of the present invention to provide compounds having herbicidal action. To be provided are in particular compounds which have high herbicidal activity, in particular even at low application rates, and who are sufficiently compatible with crop plants for commercial utilization.

These and further objects are achieved by the compounds of the formula I, defined below, and by their agriculturally suitable salts.

Accordingly, the present invention provides piperazine compounds of the general formula I

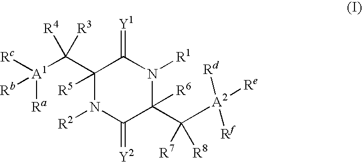

in which
A¹, A² independently of one another are aryl or heteroaryl where R^{a} is attached in the ortho-position to the point of attachment of A¹ to a carbon atom or a nitrogen atom of A¹,
Y¹ is oxygen, sulfur or a group NR^{y1} in which R^{y1} is selected from the group consisting of hydrogen, C₁-C₆-alkyl, C₃-C₆-alkenyl, C₃-C₆-alkinyl, C₃-C₆-cycloalkyl, C₃-C₆-cycloalkylmethyl, OH, C₁-C₆-alkoxy, C₃-C₆-alkenyloxy, C₃-C₆-alkinyloxy, C₃-C₆-cycloalkoxy and C₃-C₆-cycloalkylmethoxy;
Y² is oxygen, sulfur or a group NR^{y2} in which R^{y2} is selected from the group consisting of hydrogen, C₁-C₆-alkyl, C₃-C₆-alkenyl, C₃-C₆-alkinyl, C₃-C₆-cycloalkyl, C₃-C₆-cycloalkylmethyl, OH, C₁-C₆-alkoxy, C₃-C₆-alkenyloxy, C₃-C₆-alkinyloxy, C₃-C₆-cycloalkoxy and C₃-C₆-cycloalkylmethoxy;
   where the abovementioned aliphatic or cyclic moieties of the substituents Y¹ and Y² are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R^{a} is selected from the group consisting of halogen, cyano, nitro, SF₅, C₁-C₆-alkyl, C₃-C₆-cycloalkyl, C₂-C₆-alkenyl, C₅-C₈-cycloalkenyl, C₅-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₄-C₁₀-alkadienyl, C₂-C₆-alkynyl, [tri-(C₁-C₆)-alkylsilyl]-(C₂-C₆)-alkynyl, tri-(C₁-C₆)-alkylsilyl, C₇-C₈-cycloalkynyl, aryl, phenyl-(C₁-C₆)-alkyl, phenyl-(C₂-C₆)-alkenyl, phenyl-(C₂-C₆)-alkynyl, phenylsulfonyl-(C₁-C₆)-alkyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxy-carbonyl]-(C₁-C₆)-alkyl, Z¹P(O)(OR⁹)₂, Z¹P(O)(OR⁹)(R^{9a}), Z²B(OR¹⁰)₂, Z³COR¹¹, Z⁴NR¹²R¹³, Z⁵CH═N—O—R¹⁴, Z⁶OR¹⁵, Z⁷SR¹⁶, Z⁷S(O)R¹⁶ and Z⁷SO₂R¹⁶;
   where the abovementioned aliphatic, cyclic or aromatic moieties of the substituent R^{a} are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R^{b}, R^{c}, R^{d}, R^{e} and R^{f} are each independently of one another hydrogen or have one of the meanings given for R^{a}; and
   where two radicals R^{a}, R^{b} or R^{c} attached to adjacent ring atoms of A¹ or two radicals R^{d}, R^{e} or R^{f} attached to adjacent ring atoms of A² may also be straight-chain C₃-C₆-alkylene which may be partially or fully halogenated and may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy, in which a CH₂ group in C₃-C₆-alkylene may be replaced by a carbonyl group, thiocarbonyl group or sulfonyl group and in which one or two non-adjacent CH₂ groups in C₃-C₆-alkylene may in each case be replaced by oxygen, sulfur or a group NR³⁴, where R³⁴ has one of the meanings given for R¹².

R¹ and R² independently of one another are selected from the group consisting of:
cyano, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cyclo-alkenyl-(C₁-C₆)-alkyl, C₅-C₈-cycloalkynyl-(C₁-C₆)-alkyl, C₃-C₆-cycloalkyl-(C₂-C₆-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R²¹, NR²²R²³, OR²⁴, SR²⁴, S(O)R²⁵, SO₂R²⁵ and Si(R^{25a})₃;
where R¹ may additionally be hydrogen and
where the abovementioned aliphatic, cyclic or aromatic moieties of the substituents R¹ and R² independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R³ is halogen, cyano, nitro or a radical R²⁶, OR²⁷, SR²⁸, S(O)R²⁸, SO₂R²⁸, NR²⁹R³⁰ or N(OR³¹)R³²;
R⁴ is hydrogen, halogen, cyano, C₁-C₆-alkyl, C₃-C₆-cycloalkyl, C₂-C₆-alkenyl, C₅-C₈-cycloalkenyl, C₂-C₆-alkynyl, C₅-C₈-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-(C₁-C₆-alkyl or a radical COR²¹, OR²⁷, SR²⁸, S(O)R²⁸, SO₂R²⁸, NR²⁹R³⁰ or N(OR³¹)R³²,
   where the abovementioned aliphatic, cyclic or aromatic moieties of the substituent R⁴ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R⁵ is hydrogen, halogen, cyano, nitro, hydroxyl, C₁-C₈-alkyl, C₂-C₈-alkenyl, C₃-C₈-cycloalkyl, C₅-C₈-cycloalkenyl, C₂-C₈-alkynyl, C₄-C₈-alkadienyl, C₇-C₈-cycloalkynyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, tri-(C₁-C₆)-alkylsilyl, [tri-(C₁-C₆)-alkylsilyl]-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R⁶¹, Z⁸NR⁶²R⁶³, Z¹¹CH═N—O—R⁶⁴, OR⁶⁵, Z⁹SR^{65a}, Z⁹S(O)R⁶⁶, Z⁹S(O)₂R⁶⁶ or Z¹⁰P(O)(OR⁶⁷)₂)₂; or
R³ together with R⁵ is a chemical bond;
R⁶ is halogen, cyano, nitro, C₂-C₈-alkenyl, C₃-C₈-cycloalkyl, C₅-C₈-cycloalkenyl, C₂-C₈-alkynyl, C₄-C₈-alkadienyl, C₇-C₈-cycloalkynyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkyl, C₅-C₈-cycloalkenyl-(C₂-C₆)-alkynyl, C₅-C₈-cycloalkynyl-(C₂-C₆)-alkynyl, tri-(C₁-C₆)-alkylsilyl, [tri-(C₁-C₆)-alkylsilyl]-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R⁶¹, Z⁸NR⁶²R⁶³, Z¹¹CH═N—O—R⁶⁴, OR⁶⁵, Z⁹SR^{65a}, Z⁹S(O)R⁶⁶, Z⁹S(O)₂R⁶⁶ or Z¹⁰P(O)(OR⁶⁷)₂;
   where the abovementioned aliphatic, cyclic or aromatic moieties of the substituents R⁴, R⁵ and R⁶ independently of one another may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R⁷ is halogen, cyano, nitro or a radical R²⁶, OR²⁷, SR²⁸, S(O)R²⁸, SO₂R²⁸, NR²⁹R³⁰ or N(OR³¹)R³²;
R⁸ has one of the meanings given for R⁴;
R⁹, R¹⁰ and R⁶⁷ are each independently of one another hydrogen or C₁-C₆-alkyl and the radicals R¹⁰ in Z²B(OR¹⁰)₂ may together form a C₂-C₄-alkylene chain; or
R^{9a} is C₁-C₆-alkyl;
R¹¹, R⁶¹ independently of one another are hydrogen, C₁-C₆-alkyl, C₃-C₆-cycloalkyl, C₂-C₆-alkenyl, C₅-C₈-cycloalkenyl, C₂-C₆-alkynyl, C₇-C₈-cycloalkynyl, hydroxyl, C₁-C₆-alkoxy, C₃-C₆-alkenyloxy, C₃-C₆-alkynyloxy, amino, C₁-C₆-alkylamino, di-(C₁-C₆-alkyl)amino, C₁-C₆-alkoxyamino, di-(C₁-C₆-alkoxy)amino, C₁-C₆-alkylsulfonylamino, C₁-C₆-alkylaminosulfonylamino, [di-(C₁-C₆-alkyl)amino]sulfonylamino, C₃-C₆-alkenylamino, C₃-C₆-alkynylamino, N-(C₂-C₆-alkenyl)-N—(C₁-C₆-alkyl)amino, N—(C₂-C₆-alkynyl)-N—(C₁-C₆-alkyl)-amino, N—(C₁-C₆-alkoxy)-N—(C₁-C₆-alkyl)amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy, phenylamino, naphthyl or heterocyclyl;
R¹² and R⁶² independently of one another are hydrogen, C₁-C₆-alkyl, C₁-C₈-alkoxy, C₃-C₆-cycloalkyl, C₃-C₆-cycloalkyloxy, C₃-C₆-alkenyl, C₃-C₆-alkenyloxy, C₅-C₈-cycloalkenyl, C₃-C₆-alkynyl, C₃-C₆-alkynyloxy, C₇-C₈-cycloalkynyl, C₁-C₆-alkylcarbonyl, C₃-C₆-cycloalkylcarbonyl, di-(C₁-C₆-alkyl)aminocarbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, di-(C₁-C₆-alkyl)amino-sulfonyl, phenylcarbonyl, phenylaminocarbonyl, phenylsulfonyl, phenylsulfonylaminocarbonyl or heterocyclylcarbonyl;
R¹³ and R⁶³ independently of one another are hydrogen, C₁-C₆-alkyl, C₃-C₆-cycloalkyl, C₃-C₆-alkenyl, C₅-C₈-cycloalkenyl, C₃-C₆-alkynyl, C₃-C₆-alkynyloxy, C₇-C₈-cycloalkynyl, C₁-C₆-alkylcarbonyl, C₃-C₆-cycloalkylcarbonyl, di-(C₁-C₆-alkyl)aminocarbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, di-(C₁-C₆-alkyl)aminosulfonyl, phenylcarbonyl, phenylaminocarbonyl, phenylsulfonyl, phenylsulfonylaminocarbonyl or heterocyclylcarbonyl;
R¹⁴, R⁶⁴ independently of one another are hydrogen, C₁-C₆-alkyl, C₂-C₆-alkenyl or phenyl;
R¹⁵, R^{65a} independently of one another are hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl, phenyl-(C₁-C₆)-alkyl, hetaryl or hetaryl-(C₁-C₆)-alkyl;
R⁶⁵ is C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl or phenyl-(C₁-C₆)-alkyl;
R¹⁶, R⁶⁶ independently of one another are C₁-C₆-alkyl, C₁-C₆-alkoxy, phenyl or phenoxy;
Z¹, Z², Z³, Z⁴, Z⁵, Z⁶, Z⁷, Z⁸, Z⁹, Z¹⁰ and Z¹¹ independently of one another are a bond, —CH₂—, —CH₂—CH₂—, —O—CH(R¹⁷)—, —S—CH(R¹⁸)—, —S(O)—CH(R¹⁹)— or —SO₂CH(R²⁰—, in which R¹⁷, R¹⁸, R¹⁹ and R²⁰ independently of one another are hydrogen or C₁-C₆-alkyl;
R²¹ is hydrogen, C₁-C₆-alkyl, C₃-C₆-cycloalkyl, C₂-C₆-alkenyl, C₅-C₈-cycloalkenyl, C₂-C₆-alkynyl, C₇-C₈-cycloalkynyl, hydroxyl, C₁-C₆-alkoxy, C₃-C₆-alkenyloxy, C₃-C₆-alkynyloxy, amino, C₁-C₆-alkylamino, di-(C₁-C₆-alkyl)amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)-amino, N—(C₁-C₆-alkoxy)-N—(C₁-C₆-alkyl)-amino, N—(C₂-C₆-alkenyl)-N—(C₁-C₆-alkoxy)-amino, N—(C₂-C₆-alkynyl)-N—(C₁-C₆-alkoxy)-amino, phenyl, phenylamino, phenoxy, naphthyl or heterocyclyl; or
R²² and R²³ independently of one another are hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl or C₁-C₆-alkylcarbonyl; or

R²⁴ is hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl or phenyl-(C₁-C₆)-alkyl; or
R²⁵ is C₁-C₆-alkyl, C₁-C₆-alkoxy, phenyl or phenoxy;
R^{25a} is C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, phenyl or phenyl-(C₁-C₆)-alkyl; or
   where the abovementioned aliphatic, cyclic or aromatic moieties of the substituents R⁹, R^{9a}, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R^{25a}, R⁶¹, R⁶², R^{62a}, R⁶³, R⁶⁴, R⁶⁵, R^{65a}, R⁶⁶ and R⁶⁷ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R²⁶, R²⁷, R²⁸, R²⁹ and R³² independently of one another are hydrogen, C₁-C₆-alkyl, C₃-C₆-cycloalkyl, C₃-C₆-alkenyl, C₃-C₆-alkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxy-(C₁-C₆)-alkyl, C₁-C₆-alkoxycarbonyl, C₂-C₆-alkenyloxycarbonyl, C₃-C₆-alkynyloxycarbonyl, C₁-C₆-alkylaminocarbonyl, C₃-C₆-alkenylaminocarbonyl, C₃-C₆-alkynylaminocarbonyl, C₁-C₆-alkyl-sulfonylaminocarbonyl, C₁-C₆-alkylaminocarbonyl, di-(C₁-C₆-alkyl)-aminocarbonyl, N—(C₃-C₆-alkenyl)-N—(C₁-C₆-alkyl)-aminocarbonyl, N—(C₃-C₆-alkynyl)-N—(C₁-C₆-alkyl)-aminocarbonyl, N—(C₁-C₆-alkoxy)-N—(C₁-C₆-alkyl)-aminocarbonyl, N—(C₃-C₆-alkenyl)-N—(C₁-C₆-alkoxy)-aminocarbonyl, N—(C₃-C₆-alkynyl)-N—(C₁-C₆-alkoxy)-aminocarbonyl, di-(C₁-C₆-alkyl)-aminothiocarbonyl, C₁-C₆-alkylcarbonyl-C₁-C₆-alkyl, C₁-C₆-alkoxyimino-C₁-C₆-alkyl, N—(C₁-C₆-alkylamino)-imino-C₁-C₆-alkyl, N-(di-C₁-C₈-alkylamino)-imino-C₁-C⁶-alkyl or [tri-(C₁-C₄)-alkyl]silyl, where the abovementioned aliphatic or isocyclic moieties of the substituents may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl, C₁-C₄-alkylcarbonyloxy, phenyl, phenyl-C₁-C₆-alkyl, phenylcarbonyl, phenylcarbonyl-C₁-C₆-alkyl, phenoxycarbonyl, phenylaminocarbonyl, phenylsulfonylaminocarbonyl, N-(C₁-C₆-alkyl)-N-(phenyl)-aminocarbonyl, phenyl-C₁-C₆-alkylcarbonyl, heterocyclyl, heterocyclyl-C₁-C₆-alkyl, heterocyclylcarbonyl, heterocyclylcarbonyl-C₁-C₆-alkyl, heterocyclyloxycarbonyl, heterocyclylaminocarbonyl, heterocyclylsulfonylaminocarbonyl, N—(C₁-C₆-alkyl)-N-(heterocyclyl)-aminocarbonyl, or heterocyclyl-C₁-C₆-alkylcarbonyl, where the phenyl or heterocyclyl moieties of the substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   S(O)ₙR³³, where n is 1 or 2;
R³⁰ and R³¹ independently of one another are hydrogen, C₁-C₆-alkyl, C₃-C₆-cycloalkyl, C₂-C₆-alkenyl or C₂-C₆-alkynyl, where aliphatic or isocyclic moieties of the substituents R³⁰ and R³¹ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy,
   are phenyl, phenyl-C₁-C₆-alkyl, heterocyclyl or heterocyclyl-C₁-C₆-alkyl, where the phenyl or heterocyclyl moieties of the substituents may be partially or fully halogenated and/or may carry one to three of the following groups: nitro, cyano, C₁-C₄-alkyl, C₁-C₄-haloalkyl, C₁-C₄alkoxy or C₁-C₄-haloalkoxy; and
R³³ is C₁-C₆-alkyl, C₁-C₆-haloalkyl or phenyl, where the phenyl substituent may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; and
where one or 2 of the following provisions may also be met:
a) R¹ together with the radical R² or the radical R⁵ is a 1-, 2-, 3- or 4-membered carbon chain in which one carbon atom may be replaced by O, S or a group NR^{A}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
b) R¹ together with a radical R^{d}, which is attached in the ortho-position to the point of attachment of A² to a carbon atom or nitrogen atom of A², is a covalent bond or a 1-, 2-, 3- or 4-membered carbon chain in which one carbon atom may be replaced by O, S or a group NR^{B}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
c) R¹ together with the radical R⁸ or the radical R^{y1}, if present, is a 2-, 3- or 4-membered carbon chain in which one carbon atom may be replaced by O, S or a group NR^{C}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
d) R¹ together with the radical R⁶ is a 3-, 4- or 5-membered carbon chain in which one carbon atom may be replaced by O, S or a group NR^{D}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
e) R² together with the radical R⁶ is a 1-, 2-, 3- or 4-membered carbon chain in which one carbon atom may be replaced by O, S or a group NR^{E}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-alkoxy and C₁-C₄-haloalkoxy;
f) R² together with one of the radicals R^{a} or R^{b} is a covalent bond or a 1-, 2-, 3- or 4-membered carbon chain in which one carbon atom may be replaced by O, S or a group NR^{F}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
g) R² together with the radical R⁴ or the radical R^{y2}, if present, is a 2-, 3- or 4-membered carbon chain in which one carbon atom may be replaced by O, S or a group NR^{G}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
h) R² together with the radical R⁵ is a 3-, 4- or 5-membered carbon chain in which one carbon atom may be replaced by O, S or a group NR^{H}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
i) R³ together with the radical R⁵ is a 1-, 2-, 3- or 4-membered carbon chain in which one carbon atom may be replaced by O, S or a group NR^{I}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
k) R³ together with the radical R⁴ is a 2-, 3-, 4- or 5-membered carbon chain in which one carbon atom may be replaced by O, S or a group NR^{K}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
l) R⁴ together with the radical R^{a} is a 2-, 3-, 4- or 5-membered carbon chain in which one carbon atom may be replaced by O, S or a group NR^{L}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-alkoxy and C₁-C₄-haloalkoxy;
m) R⁵ together with the radical R^{a} is a 2-, 3-, 4- or 5-membered carbon chain in which one carbon atom may be replaced by O, S or a group NR^{M}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
n) R⁵ together with the radical R⁶ is a 1-, 2-, 3-, 4- or 5-membered carbon chain in which one carbon atom may be replaced by O, S or a group NR^{N}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
o) R⁶ together with a radical R^{d}, which is attached in the ortho-position to the point of attachment of A² to a carbon atom or nitrogen atom of A², is a 1-, 2-, 3- or 4-membered carbon chain in which one carbon atom may be replaced by O, S or a group NR^{o}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
p) R⁶ together with a radical R^{y2}, if present, is a 2-, 3-, 4- or 5-membered carbon chain in which one carbon atom may be replaced by O, S or a group NR^{P}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
q) R⁶ together with the radical R⁷ is a 1-, 2-, 3- or 4-membered carbon chain in which one carbon atom may be replaced by O, S or a group NR^{Q}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
r) R⁷ together with the radical R⁸ is a 2-, 3-, 4- or 5-membered carbon chain in which one carbon atom may be replaced by O, S or a group NR^{R}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
s) R⁸ together with a radical R^{d}, which is attached in the ortho-position to the point of attachment of A² to a carbon atom or nitrogen atom of A², is a 2-, 3-, 4- or 5-membered carbon chain in which one carbon atom may be replaced by O, S or a group NRS, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
t) R⁸ together with a radical R^{y2}, if present, is a 2-, 3-, 4- or 5-membered carbon chain in which one carbon atom may be replaced by O, S or a group NR^{T}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
   in which R^{A}, R^{B}, R^{C}, R^{D}, R^{E}, R^{F}, R^{G}, R^{H}, R^{I}, R^{K}, R^{L}, R^{M}, R^{N}, R^{O}, R^{P}, R^{Q}, R^{R}, R^{S} and R^{T} independently of one another are selected from the group consisting of hydrogen, cyano, C₁-C₄-alkyl, C₁-C₄-haloalkyl, phenyl and benzyl, in which the phenyl ring in phenyl or benzyl may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy;
u) R³ and R⁴ together form a keto group or a group NR^{3a} in which R^{3a} is selected from the group consisting of hydrogen, C₁-C₆-alkyl, C₁-C₆-haloalkyl, C₃-C₆-alkenyl, C₃-C₆-alkynyl, C₃-C₆-cycloalkyl, C₃-C₆-cycloalkylmethyl, OH, C₁-C₆-alkoxy, C₁-C₆-haloalkoxy, C₃-C₆-alkenyloxy, C₃-C₆-alkynyloxy, C₃-C₆-cycloalkoxy and C₃-C₆-cycloalkylmethoxy;
v) R⁷ and R⁸ together form a keto group or a group NR^{7a} in which R^{7a} is selected from the group consisting of hydrogen, C₁-C₆-alkyl, C₁-C₆-haloalkyl, C₃-C₆-alkenyl, C₃-C₆-alkynyl, C₃-C₆-cycloalkyl, C₃-C₆-cycloalkylmethyl, OH, C₁-C₆-alkoxy, C₁-C₆-haloalkoxy, C₃-C₆-alkenyloxy, C₃-C₆-alkynyloxy, C₃-C₆-cycloalkoxy and C₃-C₆-cycloalkylmethoxy;
where R⁶ may also be hydrogen, OH or C₁-C₆-alkyl which may be partially or fully halogenated and/or may carry one to three of the following groups:
   cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄-alkyl)amino]carbonyl or C₁-C₄-alkylcarbonyloxy;
if
i) at least one of the conditions a) to c), f) to m) or r) to v) is met, and/or
ii) at least one of the two groups Y¹, Y² is a group different from oxygen, and/or
iii) R⁵ is a radical different from hydrogen, hydroxyl or C₁-C₆-alkyl, where C₁-C₆-alkyl is unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄-alkyl)amino]carbonyl or C₁-C₄-alkylcarbonyloxy;
and/or
iv) one or both of the radicals R⁷, R⁸ is/are a radical different from hydrogen, hydroxyl, C₁-C₆-alkyl, C₁-C₆-alkoxy, where C₁-C₆-alkyl and C₁-C₆-alkoxy are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄-alkyl)amino]carbonyl or C₁-C₄-alkylcarbonyloxy;
and/or
v) one or both of the radicals R¹, R² is/are SR²⁴, S(O)R²⁵, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl-(C₂-C₆)-alkenyl, phenyl-(C₂-C₆)-alkynyl, heterocyclyl-(C₂-C₆)-alkenyl or heterocyclyl-(C₂-C₆)-alkynyl, where the abovementioned aliphatic, cyclic or aromatic moieties of the substituents R¹ and R² independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
and/or
vi) R^{a} is SF₅, Z¹P(O)(OR⁹)(R^{9a}), C₃-C₆-cycloalkyl-(C₁-C₆)-alkyl, C₅-C₈-cyclo-alkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl-(C₂-C₆)-alkynyl, heterocyclyl-(C₂-C₆)-alkenyl or heterocyclyl-(C₂-C₆)-alkynyl, where the abovementioned aliphatic, cyclic or aromatic moieties of the substituent R^{a} independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
and where R⁶ is not C₁-C₆-alkoxy which may be partially or fully halogenated and/or may carry one to three of the following groups:
   cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄-alkyl)amino]carbonyl or C₁-C₄-alkylcarbonyloxy;
if R³ together with R⁵ is a chemical bond;
   and their salts.

The present invention also provides the use of piperazine compounds of the general formula I or the agriculturally useful salts of piperazine compounds of the formula I as herbicides, i.e. for controlling harmful plants.

The present invention also provides compositions comprising at least one piperazine compound of the formula I or an agriculturally useful salt of I and auxiliaries customary for formulating crop protection agents.

The present invention furthermore provides a method for controlling unwanted vegetation where a herbicidally effective amount of at least one piperazine compound of the formula I or an agriculturally useful salt of I is allowed to act on plants, their seeds and/or their habitat.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

Depending on the substitution pattern, the compounds of the formula I may comprise one or more centers of chirality, in which case they are present as enantiomer or diastereomer mixtures. The invention provides both the pure enantiomers or diastereomers and their mixtures.

If R³ together with R⁵ is a chemical bond, compounds of the formula I may be present as E isomer or Z isomer with respect to the exocyclic double bond thus formed. The invention provides both the pure E isomers and Z isomers and their mixtures.

The compounds of the formula I may also be present in the form of their agriculturally useful salts, the nature of the salt generally being immaterial. Suitable salts are, in general, the cations or the acid addition salts of those acids whose cations and anions, respectively, have no adverse effect on the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents of the compounds according to the invention are collective terms for individual enumerations of the specific group members. All hydrocarbon chains, such as
alkyl, haloalkyl, and also the alkyl moieties in cyanoalkyl, alkoxy, haloalkoxy, alkylthio, haloalkylthio, alkylsulfinyl, haloalkylsulfinyl, alkylsulfonyl, haloalkylsulfonyl, N-alkylaminosulfonyl, N,N-dialkylaminosulfonyl, dialkylamino, N-alkylsulfonylamino, N-haloalkylsulfonylamino, N-alkyl-N-alkylsulfonylamino, N-alkyl-N-haloalkylsulfonylamino, alkylcarbonyl, haloalkylcarbonyl, alkoxycarbonyl, haloalkoxycarbonyl, alkylcarbonyloxy, alkylaminocarbonyl, dialkylaminocarbonyl, dialkylaminothiocarbonyl, alkoxyalkyl, dialkoxyalkyl, alkylthioalkyl, dialkylaminoalkyl, dialkylhydrazinoalkyl, alkyliminooxyalkyl, alkylcarbonylalkyl, alkoxyiminoalkyl, N-(alkylamino)-iminoalkyl, N-(dialkylamino)-iminoalkyl, alkoxycarbonylalkyl, dialkylaminocarbonylalkyl, phenylalkenylcarbonyl, heterocyclylalkenylcarbonyl, N-alkoxy-N-alkylaminocarbonyl, N-alkyl-N-phenylaminocarbonyl, N-alkyl-N-heterocyclylaminocarbonyl, phenylalkyl, heterocyclylalkyl, phenylcarbonylalkyl, heterocyclylcarbonylalkyl, dialkylaminoalkoxycarbonyl, alkoxyalkoxycarbonyl, alkenylcarbonyl, alkenyloxycarbonyl, alkenylaminocarbonyl, N-alkenyl-N-alkylaminocarbonyl, N-alkenyl-N-alkoxyaminocarbonyl, alkynylcarbonyl, alkynyloxycarbonyl, alkynylaminocarbonyl, N-alkynyl-N-alkylaminocarbonyl, N-alkynyl-N-alkoxyaminocarbonyl, alkenyl, alkynyl, haloalkenyl, haloalkynyl and alkoxyalkoxy moieties

may be straight-chain or branched. The prefix Cₙ—Cₘ— indicates the respective carbon number of the hydrocarbon moiety. Unless indicated otherwise, halogenated substituents preferably carry one to five identical or different halogen atoms, in particular fluorine atoms or chlorine atoms.

The term halogen denotes in each case fluorine, chlorine, bromine or iodine.

Examples of other meanings are:

alkyl and also the alkyl moieties, for example, in alkoxy, alkylthio, alkylsulfinyl and alkylsulfonyl, alkylcarbonyl, alkylamino, trialkylsilyl, phenylalkyl, phenylsulfonylalkyl, heterocyclylalkyl: saturated straight-chain or branched hydrocarbon radicals having one or more carbon atoms, for example 1 to 2, 1 to 4 or 1 to 6 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl. In one embodiment according to the invention, alkyl denotes small alkyl groups such as C₁-C₄-alkyl. In another embodiment according to the invention, alkyl denotes relatively large alkyl groups such as C₅-C₆-alkyl.

Haloalkyl: an alkyl radical as mentioned above whose hydrogen atoms are partially or fully substituted by halogen atoms such as fluorine, chlorine, bromine and/or iodine, for example chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl and nonafluorobutyl.

Cycloalkyl and also the cycloalkyl moieties, for example, in cycloalkoxy or cycloalkylcarbonyl: monocyclic saturated hydrocarbon groups having three or more carbon atoms, for example 3 to 6 carbon ring members, such as cyclopropyl, cyclobutyl, cyclopentyl and cyclohexyl.

Alkenyl and also alkenyl moieties, for example, in phenyl-(C₂-C₆)-alkenyl or alkenylamino: monounsaturated straight-chain or branched hydrocarbon radicals having two or more carbon atoms, for example 2 to 4, 2 to 6, or 3 to 6 carbon atoms, and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-tri-methyl-2-propenyl, 1-ethyl-1-methyl-2-propenyl, 1-ethyl-2-methyl-1-propenyl, 1-ethyl-2-methyl-2-propenyl.

In one embodiment according to the invention, alkenyl groups such as C₂-C₆-alkenyl are employed. In another embodiment according to the invention, use is made of alkenyl groups such as C₃-C₆-alkenyl.

Cycloalkenyl and also cycloalkenyl moieties in cycloalkenylalkyl, cycloalkenylalkenyl and cycloalkenylakynyl: monocyclic, monounsaturated hydrocarbon groups having three or more carbon atoms, for example 5 to 8, preferably 5 to 6, carbon ring members, such as cyclopenten-1-yl, cyclopenten-3-yl, cyclohexen-1-yl, cyclohexen-3-yl, cyclohexen-4-yl.

Alkynyl and also alkynyl moieties, for example, in [tri-(C₁-C₆)-alkylsilyl-(C₂-C₆)-alkynyl or alkynylamino: straight-chain or branched hydrocarbon groups having two or more carbon atoms, for example 2 to 4, 2 to 6, or 3 to 6 carbon atoms, and one or two triple bonds in any position, but not adjacent to one another, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Cycloalkynyl and also cycloalkynyl moieties in cycloalkynylalkyl, cycloalkynylalkenyl and cycloalkynylalkynyl: monocyclic hydrocarbon groups having three or more carbon atoms, for example 7 to 8, carbon ring members and one triple bond, such as cycloheptyn-1-yl, cycloheptyn-3-yl, cycloheptyn-4-yl.

C₄-C₁₀-alkadienyl: doubly unsaturated straight-chain or branched hydrocarbon radicals having four or more carbon atoms and two double bonds in any (but non-adjacent) position, for example 4 to 10 carbon atoms and two double bonds in any position, but not adjacent to one another, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl.

Alkoxy or alkoxy moieties, for example, in phenylalkoxy, alkoxyamino, alkoxycarbonyl: alkyl, as defined above, which is attached via an oxygen atom: for example methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In one embodiment according to the invention, small alkoxy groups such as C₁-C₄-alkoxy are employed. In another embodiment according to the invention, use is made of relatively large alkoxy groups such as C₅-C₆-alkoxy.

Alkenyloxy: alkenyl as mentioned above which is attached via an oxygen atom, for example C₃-C₆-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyloxy, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy. In one embodiment according to the invention, small alkenyloxy groups such as C₃-C₄-alkenyloxy are employed. In another embodiment according to the invention, use is made of relatively large alkenyloxy groups such as C₅-C₆-alkenyloxy.

Alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy. In one embodiment according to the invention, small alkynyloxy groups such as C₃-C₄-alkynyloxy are employed. In another embodiment according to the invention, use is made of relatively large alkynyloxy groups such as C₅-C₆-alkynyloxy.

Alkylthio: alkyl as defined above which is attached via a sulfur atom.

Alkylsulfinyl: alkyl as defined above which is attached via an SO group.

Alkylsulfonyl: alkyl as defined above which is attached via an S(O)₂ group.

Alkylcarbonyl: alkyl as defined above which is attached via a (C═O) group, for example methylcarbonyl, ethylcarbonyl, propylcarbonyl, 1-methylethylcarbonyl, butylcarbonyl, 1-methylpropylcarbonyl, 2-methylpropylcarbonyl or 1,1-dimethylethylcarbonyl, pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2,-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl.

Alkenylcarbonyl: alkenyl as defined above which is attached via a (C═O) group, for example 1-ethenylcarbonyl.

Alkynylcarbonyl: alkynyl as defined above which is attached via a (C═O) group, for example 1-propynylcarbonyl.

Heterocyclyl: a mono- or bicyclic saturated, partially unsaturated or aromatic heterocyclic ring having three or more, for example 3 to 10, ring atoms:
for example a monocyclic 3-, 4-, 5-, 6-or 7-membered heterocyclic ring which contains one to four identical or different heteroatoms selected from the group consisting of oxygen, sulfur and nitrogen and which may be attached via carbon or nitrogen, for example
3- or 4-membered saturated or unsaturated rings attached via carbon, such as 2-oxiranyl, 2-oxetanyl, 3-oxetanyl, 2-aziridinyl, 3-thiethanyl, 1-azetidinyl, 2-azetidinyl;
5-membered saturated rings attached via carbon, such as
tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydro-pyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;
6-membered saturated rings attached via carbon, such as:
tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;
5-membered saturated rings attached via nitrogen, such as:
tetrahydropyrrol-1-yl, tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;
6-membered saturated rings attached via nitrogen, such as:
piperidin-1-yl, hexahydropyrimidin-1-yl, hexahydropyrazin-1-yl, hexahydropyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl, tetrahydro-1,2-oxazin-2-yl;
5-membered partially unsaturated rings attached via carbon, such as:
2,3-dihydrofuran-2-yl, 2,3-dihydrofuran-3-yl, 2,5-dihydrofuran-2-yl, 2,5-dihydrofuran-3-yl, 4,5-dihydrofuran-2-yl, 4,5-dihydrofuran-3-yl, 2,3-dihydrothien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, Δ³-1,2-dithiol-3-yl, Δ³-1,2-dithiol-4-yl, Δ³-1,2-dithiol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydrooxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydro-oxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl, 1,2,3-Δ²-oxadiazolin-4-yl, 1,2,3-Δ²-oxadiazolin-5-yl, 1,2,4-Δ⁴-oxadiazolin-3-yl, 1,2,4-Δ⁴-oxadiazolin-5-yl, 1,2,4-Δ²-oxadia-zolin-3-yl, 1,2,4-Δ²-oxadiazolin-5-yl, 1,2,4-Δ³-oxadiazolin-3-yl, 1,2,4-Δ³-oxadiazolin-5-yl, 1,3,4-Δ²-oxadiazolin-2-yl, 1,3,4-Δ²-oxadiazolin-5-yl, 1,3,4-Δ³-oxadiazolin-2-yl, 1,3,4-oxadiazolin-2-yl, 1,2,4-Δ⁴-thiadiazolin-3-yl, 1,2,4-Δ⁴-thiadiazolin-5-yl, 1,2,4-Δ³-thiadiazolin-3-yl, 1,2,4-Δ³-thiadiazolin-5-yl, 1,2,4-Δ²-thiadiazolin-3-yl, 1,2,4-Δ²-thiadiazolin-5-yl, 1,3,4-Δ²-thiadiazolin-2-yl, 1,3,4-Δ²-thiadiazolin-5-yl, 1,3,4-Δ³-thiadiazolin-2-yl, 1,3,4-thiadiazolin-2-yl, 1,2,3-Δ²-triazolin-4-yl, 1,2,3-Δ²-triazolin-5-yl, 1,2,4-Δ²-triazolin-3-yl, 1,2,4Δ²-triazolin-5-yl, 1,2,4-Δ³-triazolin-3-yl, 1,2,4-Δ³-triazolin-5-yl, 1,2,4-Δ¹-triazolin-2-yl, 1,2,4-triazolin-3-yl, 3H-1,2,4-dithiazol-5-yl, 2H-1,3,4-dithiazol-5-yl, 2H-1,3,4-oxathiazol-5-yl;
6-membered partially unsaturated rings attached via carbon, such as:
2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydropyran-6-yl, 2H-3,4-dihydrothiopyran-5-yl, 2H-3,4-dihydrothiopyran-4-yl, 2H-3,4-dihydropyran-3-yl, 2H-3,4-dihydropyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetrahydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl-, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 3,4-dihydropyridin-2-yl, 3,4-dihydropyridin-3-yl, 3,4-dihydropyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-di-hydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydropyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetrahydropyridazin-5-yl, 1,2,5,6-tetrahydropyridazin-6-yl, 1,2,3,6-tetrahydropyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5-6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydropyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,2-oxazin-3-yl, 2H-1,2-oxazin-4-yl, 2H-1,2-oxazin-5-yl, 2H-1,2-oxazin-6-yl, 2H-1,2-thiazin-3-yl, 2H-1,2-thiazin-4-yl, 2H-1,2-thiazin-5-yl, 2H-1,2-thiazin-6-yl, 4H-1,2-oxazin-3-yl, 4H-1,2-oxazin-4-yl, 4H-1,2-oxazin-5-yl, 4H-1,2-oxazin-6-yl, 4H-1,2-thiazin-3-yl, 4H-1,2-thiazin-4-yl, 4H-1,2-thiazin-5-yl, 4H-1,2-thiazin-6-yl, 6H-1,2-oxazin-3-yl, 6H-1,2-oxazin-4-yl, 6H-1,2-oxazin-5-yl, 6H-1,2-oxazin-6-yl, 6H-1,2-thiazin-3-yl, 6H-1,2-thiazin-4-yl, 6H-1,2-thiazin-5-yl, 6H-1,2-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl;
5-membered partially unsaturated rings attached via nitrogen, such as:
2,3-dihydro-1H-pyrrol-1-yl, 2,5-dihydro-1H-pyrrol-1-yl, 4, 5-dihydro-1H-pyrazol-1-yl, 2,5-dihydro-1H-pyrazol-1-yl, 2,3-dihydro-1H-pyrazol-1-yl, 2, 5-dihydroisoxazol-2-yl, 2,3-dihydroisoxazol-2-yl, 2,5-dihydroisothiazol-2-yl, 2,3-dihydroisoxazol-2-yl, 4,5-dihydro-1H-imidazol-1-yl, 2,5-dihydro-1H-imidazol-1-yl, 2,3-dihydro-1H-imidazol-1-yl, 2,3-dihydrooxazol-3-yl, 2,3-dihydrothiazol-3-yl, 1,2,4-Δ⁴-oxadiazolin-2-yl, 1,2,4-Δ²-oxadiazolin-4-yl, 1,2,4-Δ³-oxadiazolin-2-yl, 1,3,4-Δ²-oxadiazolin-4-yl, 1,2,4-Δ⁵-thiadiazolin-2-yl, 1,2,4-Δ³-thiadiazolin-2-yl, 1,2,4-Δ²-thiadiazolin-4-yl, 1,3,4-Δ²-thiadiazolin-4-yl, 1,2,3-Δ²-triazolin-1-yl, 1,2,4-Δ²-triazolin-1-yl, 1,2,4-Δ²-triazolin-4-yl, 1,2,4-Δ³-triazolin-1-yl, 1,2,4-Δ¹-triazolin-4-yl;
6-membered partially unsaturated rings attached via nitrogen, such as:
1,2,3,4-tetrahydropyridin-1-yl, 1,2,5,6-tetrahydropyridin-1-yl, 1,4-dihydropyridin-1-yl, 1,2-dihydropyridin-1-yl, 2H-5,6-dihydro-1,2-oxazin-2-yl, 2H-5,6-dihydro-1,2-thiazin-2-yl, 2H-3,6-dihydro-1,2-oxazin-2-yl, 2H-3,6-dihydro-1,2-thiazin-2-yl, 2H-3,4-dihydro-1,2-oxazin-2-yl, 2H-3,4-dihydro-1,2-thiazin-2-yl, 2,3,4,5-tetrahydro-pyridazin-2-yl, 1,2,5,6-tetrahydropyridazin-1-yl, 1,2,5,6-tetrahydropyridazin-2-yl, 1,2,3,6-tetrahydropyridazin-1-yl, 3,4,5,6-tetrahydropyrimidin-3-yl, 1,2,3,4-tetra-hydropyrazin-1-yl, 1,2,3,4-tetrahydropyrimidin-1-yl, 1,2,3,4-tetrahydropyrimidin-3-yl, 2,3-dihydro-1,4-thiazin-4-yl, 2H-1,2-oxazin-2-yl, 2H-1,2-thiazin-2-yl, 4H-1,4-oxazin-4-yl, 4H-1,4-thiazin-4-yl, 1,4-dihydropyridazin-1-yl, 1,4-dihydropyrazin-1-yl, 1,2-dihydropyrazin-1-yl, 1,4-dihydropyrimidin-1-yl or 3,4-dihydropyrimidin-3-yl;
5-membered heteroaromatic rings, attached via carbon, having generally 1, 2, 3 or 4 nitrogen atoms or one oxygen or sulfur atom and, if appropriate, 1, 2 or 3 nitrogen atoms as ring members, such as:
2-furyl, 3-furyl, 2-thienyl, 3-thienyl, pyrrol-2-yl, pyrrol-3-yl, pyrazol-3-yl, pyrazol-4-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, oxazol-2-yl, oxazol-4-yl, oxazol-5-yl, thiazol-2-yl, thiazol-4-yl, thiazol-5-yl, 1,2,3-oxadiazol-4-yl, 1,2,3-oxadiazol-5-yl, 1,2,4-oxadiazol-3-yl, 1,2,4,-oxadiazol-5-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, tetrazol-5-yl;
6-membered heteroaromatic rings, attached via carbon, having generally 1, 2, 3 or 4 nitrogen atoms as ring members, such as:
pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl, 1,3,5-triazin-2-yl, 1,2,4-triazin-3-yl, 1,2,4-triazin-5-yl, 1,2,4-triazin-6-yl, 1,2,4,5-tetrazin-3-yl;
5-membered heteroaromatic rings, attached via nitrogen, having generally 1, 2, 3 or 4 nitrogen atoms as ring members, such as:
pyrrol-1-yl, pyrazol-1-yl, Imidazol-1-yl, 1,2,3-triazol-1-yl, 1,2,4-triazol-1-yl, tetrazol-1-yl;
or
   a bicyclic heterocycle which comprises one of the above-mentioned 5- or 6-membered heterocyclic rings and a further fused-on saturated, unsaturated or aromatic carbocycle, for example a benzene, cyclohexane, cyclohexene or cyclohexadiene ring, or a further fused-on 5- or 6-membered heterocyclic ring, where the latter may likewise be saturated, unsaturated or aromatic.

A sulfur atom in the heterocycles mentioned may be oxidized to S═O or S(═O)₂.

Accordingly, hetaryl or heteroaryl is a 5- or 6-membered heteroaromatic radical which has 1, 2, 3 or 4 identical or different heteroatoms selected from the group consisting of oxygen, sulfur and nitrogen as ring members, which may be attached via carbon or nitrogen and which, together with a further fused-on benzene ring or a 5- to 6-membered heteroaromatic may form a bicyclic ring system. Examples of hetaryl are the above-mentioned 5- and 6-membered heteroaromatic rings attached via carbon, the above-mentioned 5-membered heteroaromatic rings attached via nitrogen and bicyclic heteroaramatic radicals such as quinolinyl, isoquinolinyl, quinazolinyl, quinoxalinyl, indolyl, benzothienyl, benzofuryl, benzoxazolyl, benzothiazolyl, benzimidazolyl, benzopyrazolyl, benzotriazole, indolizinyl, 1,2,4-triazolo[1,5-a]pyrimidinyl, 1,2,4-triazolo[4,3-a]pyridinyl, pyrazolo[3,4-b]pyridinyl, 1,2,4-triazolo[1,5-a]pyridinyl, imidazo[1,2-a]pyridyl, imidazo[3,4-a]pyrimidinyl, and the like.

Aryl: a mono- or polycyclic aromatic carbocycle, for example a mono- or bicyclic or a mono- to tricyclic aromatic carbocycle having 6 to 14 ring members, such as, for example, phenyl, naphthyl or anthracenyl.

Arylalkyl: an aryl radical attached via an alkylene group, in particular via a methylene, 1,1-ethylene or 1,2-ethylene group, for example benzyl, 1-phenylethyl and 2-phenylethyl.

Phenylalkenyl: a phenyl radical which is attached via an alkenylene group, in particular via a 1,1-ethenylene group (vinylidene) or 1,2-ethenylene group, for example 1-styryl and 2-styryl.

Phenylalkynyl: a phenyl radical which is attached via an alkynylene group, in particular via a 1,2-ethynylene group.

Heterocyclylalkyl and also hetarylalkyl: a heterocyclyl- or hetaryl radical attached via an alkylene group, in particular via a methylene, 1,1-ethylene or 1,2-ethylene group.

Heterocyclylalkenyl and also hetarylalkenyl: a heterocyclyl or hetary radical which is attached via an alkenylene group, in particular via a 1,1-ethenylene group (vinylidene) or 1,2-ethenylene group.

Heterocyclylalkynyl and also hetarylalkynyl: a heterocyclyl or hetary radical which is attached via an alkynylene group, in particular via a 1,2-ethynylene group.

In a particular embodiment, the variables of the compounds of the formula I have the meanings below, these meanings—both on their own and in combination with one another—being particular embodiments of the compounds of the formula I:

Independently of one another, A¹ and A² are selected from the group consisitng of phenyl, furyl, thienyl and pyridinyl. In particular, A¹ is phenyl or pyridinyl. A² is in particular phenyl or thienyl.

Y¹ and Y² are in particular O.

A particularly preferred embodiment of the invention relates to compounds of the formula I and their salts in which A¹ and A² are each phenyl. From among these, preference is given to those compounds in which Y¹ and Y² are O. Hereinbelow, these compounds are also referred to as compounds of the formula I′:

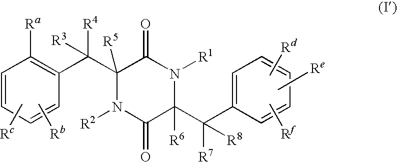

In the formula I′, R¹, R², R³, R⁴, R⁵, R⁶, R⁷, R⁸, R^{a}, R^{b}, R^{c}, R^{d}, R^{e}, and R^{f} have one of the meanings mentioned above and in particular one of the meanings mentioned below as being preferred.

Preferably, R^{a} in the compounds of the formula I is selected from the group consisting of halogen, cyano, nitro, C(═O)—R¹¹, phenyl and a 5- or 6-membered heterocyclic radical which has 1, 2, 3 or 4 heteroatoms selected from the group consisitng of O, N and S as ring atoms, where phenyl and the heterocyclic radical are unsubstituted or may have 1, 2, 3 or 4 substituents independently of one another selected from the group consisting of halogen, CN, NO₂, C₁-C₄-haloalkyl, C₁-C₄-alkoxy and C₁-C₄-haloalkoxy, where

R¹¹ is hydrogen, C₁-C₆-alkyl, hydroxyl, C₁-C₆-alkoxy, C₃-C⁶-alkenyloxy, C₃-C₆-alkynyloxy, amino, C₁-C₆-alkylamino, [di-(C₁-C₆)-alkyl]amino, C₁-C₆-alkoxyamino, N—C₁-C₆-alkoxy-N—C₁-C₆-alkylamino, C₁-C₆-alkylsulfonylamino, C₁-C₆-alkylamino-sulfonylamino, [di-(C₁-C₆)-alkylamino]sulfonylamino, phenyl, phenoxy, phenylamino, naphthyl or heterocyclyl, and

the abovementioned aliphatic, cyclic or aromatic moieties of the substituent R¹¹ may be partially or fully halogenated.

R^{a} is in particular cyano, nitro or a 5- or 6-membered heteroaromatic radical, as defined above, which has preferably either 1, 2 or 3 nitrogen atoms or 1 oxygen or 1 sulfur atom and optionally 1 or 2 nitrogen atoms as ring members and which is unsubstituted or may have 1 or 2 of the substituents mentioned above.

In a first preferred embodiment of the invention, R^{a} is cyano or nitro.

In a further preferred embodiment of the invention, R^{a} is a 5- or 6-membered heteroaromatic radical, as defined above, which has preferably either 1, 2, 3 or 4 nitrogen atoms or 1 oxygen or 1 sulfur atom and optionally 1 or 2 nitrogen atoms as ring members and which is unsubstituted or may have 1 or 2 of the substituents mentioned above. Examples of preferred heteroaromatic radicals are pyridazin-3-yl, pyridazin-4-yl, pyrimidin-2-yl, pyrimidin-4-yl, pyrimidin-5-yl, pyrazin-2-yl, 2-furyl, 3-furyl, 2-thienyl, 3-thienyl, pyrazol-1-yl, pyrazol-3-yl, pyrazol-4-yl, Isoxazol-3-yl, isoxazol-4-yl, isoxazol-5-yl, isothiazol-3-yl, isothiazol-4-yl, isothiazol-5-yl, imidazol-1-yl, imidazol-2-yl, imidazol-4-yl, oxazol-2-yl, oxazol-4-yl, oxazol-5-yl, thiazol-2-yl, thiazol-4-yl and thiazol-5-yl, in particular heteroaromatic radicals attached via carbon, such as pyrazol-3-yl, imidazol-5-yl, oxazol-2-yl, thiazol-2-yl, thiazol-4-yl, thiazol-5-yl, pyridin-2-yl, pyridin-3-yl, pyridin-4-yl, pyrimidin-2-yl, pyrimidin-4-yl, pyrimidin-5-yl, pyridazin-4-yl, pyrazin-2-yl, [1H]-tetrazol-5-yl and [2H]-tetrazol-5-yl, where the heterocycles mentioned here in an exemplary manner may have 1 or 2 of the substituents mentioned above. Preferred substituents are in particular F, Cl, CN, nitro, methyl, ethyl, methoxy, ethoxy, difluoromethoxy, trifluoromethoxy and trifluoromethyl.

Preference is likewise given to compounds of the general formula I and their salts in which R^{a} is halogen and in particular chlorine or bromine.

In the compounds of the formula I, R^{b} is preferably selected from the group consisting of hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR²¹ in which R²¹ is C₁-C₄-alkyl or C₁-C₄-haloalkyl and n is 0, 1 or 2.

Particularly preferably, R^{b} is hydrogen, fluorine, chlorine, C₁-C₂-alkyl, C₁-C₂-fluoroalkyl, ethenyl, C₁-C₂-alkoxy or C₁-C₂-fluoroalkoxy, in particular fluorine, chlorine, methyl, ethyl, methoxy, ethenyl or trifluoromethoxy. R^{b} is in particular hydrogen, fluorine or chlorine.

From among the compounds of the formula I in which R^{b} is different from hydrogen, preference is given to those compounds in which R^{b} is located in the ortho-position to the point of attachment of the phenyl ring.

In a particularly preferred embodiment, R^{b} is halogen, in particular chlorine or fluorine, which is located in the ortho-position to the point of attachment of the phenyl ring.

In the compounds of the formula I, R^{c} is preferably hydrogen or halogen, in particular chlorine or fluorine.

From among the compounds of the formula I in which R^{c} is halogen, preference is given to those compounds in which R^{c} is located in the para-position to group R^{a}.

In another embodiment, which is likewise preferred, R^{c} is hydrogen.

In the compounds of the formula I, R^{d} and R^{e} are preferably independently of one another selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy.

From among the compounds of the formula I in which R^{d} is a radical different from hydrogen, preference is given to those compounds in which R^{d} is located in the paraposition to the group CR⁷R⁸.

From among the compounds of the formula I in which R^{d} is a radical different from hydrogen, preference is given to those compounds in which R^{d} is halogen, in particular fluorine or chlorine. In another embodiment, which is likewise preferred, R^{d} is hydrogen.

In the compounds of the general formula I, R^{e} is preferably hydrogen.

In the compounds of the general formula I, R^{f} is preferably hydrogen.

A particularly preferred embodiment of the invention relates to compounds of the formula I′ and their salts in which R^{b} is located in the ortho-position to the point of attachment of the phenyl ring, R^{c} is located in the para-position to group R^{a}, R^{d} is located in the para-position to group CR⁷R⁸ and R^{e} and R^{f} are each hydrogen. From among these, preference is given to those compounds in which Y¹ and Y² are O. Hereinbelow, these compounds are also referred to as compounds of the formula I.a:

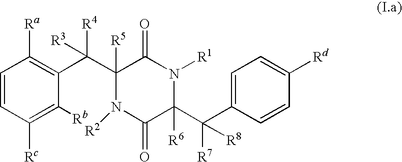

In formula I.a, R¹, R², R³, R⁴, R⁵, R⁶, R⁷, R⁸, R^{a}, R^{b}, R^{c} and R^{d} have one of the meanings mentioned above or below as being preferred.

In the compounds of the formula I, R¹ is preferably selected from the group consisting of hydrogen, C₁-C₆-alkyl and C₁-C₆-alkylcarbonyl. R¹ is in particular hydrogen or methyl.

In the compounds of the formula I, R² is preferably selected from the group consisting of C₁-C₆-alkyl and C₁-C₆-alkylcarbonyl. R² is in particular methyl.

In the compounds of the formula I, R³ is preferably R²⁶ or OR²⁷, where R²⁶ and R²⁷ independently of one another are selected from the group consisting of hydrogen, C₁-C₆-alkyl C₁-C₆-alkylcarbonyl, phenyl-C₁-C₆-alkyl, phenylcarbonyl, where the abovementioned aliphatic or aromatic moieties of the substituents may be partially or fully halogenated, or a group SO₂R³³, where R³³ is C₁-C₆-alkyl or phenyl, and where the phenyl substituent may be partially or fully halogenated and/or may carry one to three C₁-C₆-alkyl groups.

Particularly preferably, R³ in the compounds of the general formula I is hydrogen, C₁-C₆-alkyl, phenyl-C₁-C₆-alkoxy or C₁-C₆-alkylsulfonyl. Very particularly preferably, R³ is hydrogen.

In the compounds of the general formula I, R⁴ is preferably hydrogen.

Preference is likewise given to compounds of the formula I in which R³ and R⁴ together with the carbon atom to which they are attached are a carbonyl group.

In the compounds of the general formula I, R⁵ is preferably hydrogen, hydroxyl or C₁-C₆-alkyl and in particular methyl or hydroxyl.

A preferred embodiment of the invention relates to compounds of the general formula I in which R³ together with R⁵ is a chemical bond. These compounds are described by the formula I-A below:

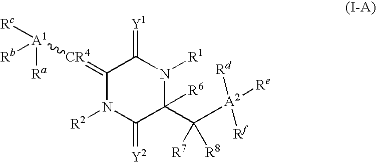

In formula I-A, A¹, A², R¹, R², R⁴, R⁶, R⁷, R⁸, R^{a}, R^{b}, R^{c}, R^{d}, R^{e} and R^{f} have one of the meanings mentioned above and in particular one of the meanings mentioned above or below as being preferred. From among these compounds I-A, particular preference is given to compounds having the features of the general formula I′ (compounds of the formula I′-A).

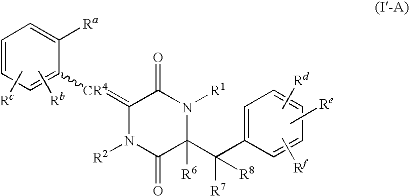

In formula I′-A, R¹, R², R⁴, R⁶, R⁷, R⁸, R^{a}, R^{b}, R^{c}, R^{d}, R^{e} and R^{f} have one of the meanings mentioned above and in particular one of the meanings mentioned above or below as being preferred.

From among these compounds I′-A, particular preference is given to compounds having the features of the general formula I.a. Hereinbelow, these compounds are also referred to as compounds of the formula I-A.a:

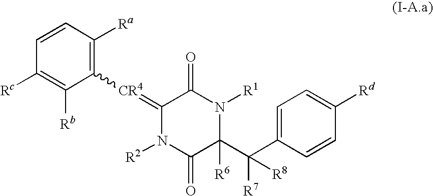

In formula I-A.a, R¹, R², R⁴, R⁶, R⁷, R⁸, R^{a}, R^{b}, R^{c} and R^{d} preferably have one of the meanings mentioned above or below as being preferred.

From among the compounds of the formulae I-A, I′-A and I-A.a, preference is given to those compounds in which the exo double bond at the piperazine ring has the (Z) configuration. Preference is likewise given to mixtures of the (E) isomers with the (Z) isomer in which the Z isomer is present in excess, in particular to isomer mixtures having an E/Z ratio of not more than 1:2, in particular not more than 1:5.

In the compounds of the formula I, the 6-position of the piperazine ring, i.e. the position in which the radical R⁶ is attached, has a center of chirality. From among the compounds of the general formula I, preference is given to the compounds of the formula I-S as compared to their enantiomer I-R:

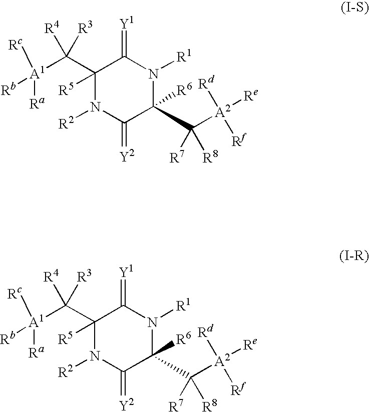

In the formulae I-S and I-R, A¹, A², R¹, R², R³, R⁴, R⁶, R⁷, R⁸, R^{a}, R^{b}, R^{c}, R^{d}, R^{e}, R^{f}, Y¹ and Y² have one of the meanings mentioned above and in particular one of the meanings mentioned above or below as being preferred. Preference is likewise given to mixtures of the compound I-S with the compound I-R in which the compounds I-S is present in excess, in particular to mixtures having a ratio of I-S to I-R of at least 2:1, in particular at least 5:1. Also suitable are mixtures having a smaller ratio of I-S to I-R, for example racemic mixtures.

A further embodiment of the invention relates to compounds of the formula I in which R⁵ is not together with R³ a chemical bond. Hereinbelow, these compounds are also referred to as compounds I-B.

The compounds of the formula I-B in which R⁵ is not together with R³ a chemical bond have in each case a center of chirality at the carbon atoms in the 3- and/or the 6-position of the piperazine ring. Accordingly, these compounds can be present in 4 different stereoisomeric forms, as shown below:

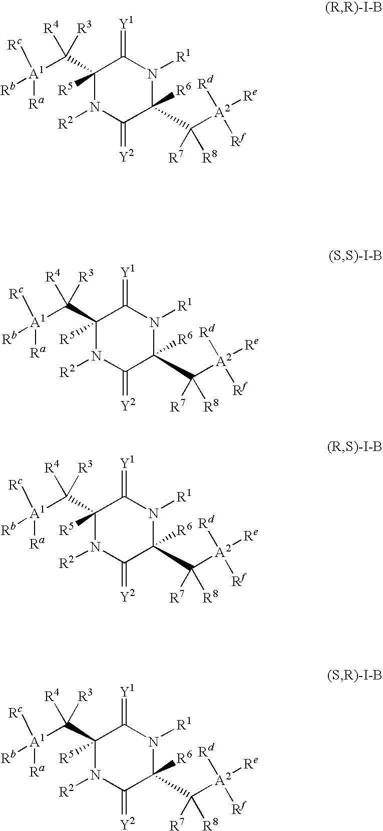

In the formulae (R,R)-I-B, (S,S)-I-B, (R,S)-I-B and (S,R)-I-B , Y¹, Y², A¹, A², R¹, R², R³, R⁴, R⁶, R⁷, R⁸, R^{a}, R^{b}, R^{c}, R^{d}, R^{e} and R^{f} have one of the meanings mentioned above or below as being preferred. From among these, preference is given to those compounds of the formula I-B in which the benzylic groups in the 3- and the 6-position have a cis arrangement with respect to the piperazine ring, i.e. generally the S,S enantiomer (S,S)-I-B and the R,R enantiomer (R,R)-I-B and also their mixtures. Preference is likewise given to mixtures of the cis compound(s) with the trans compound(s) in which the cis compound(s) is/are present in excess, in particular to cis/trans mixtures having a cis/trans ratio of at least 2:1, in particular at least 5:1.

A particularly preferred embodiment of the invention relates to the enantiomer of the formula (S,S)-I-B, and also to enantiomer mixtures and diastereomer mixtures of I-B in which the enantiomer (S,S)-I-B is the main component and is preferably present in a proportion of at least 70%, in particular at least 80% and especially at least 90% of the compound I-B. Preference is also given to the agriculturally suitable salts of the enantiomers (S,S)-I-B and to enantiomer mixtures and diastereomer mixtures of the salts in which the enantiomer (S,S)-I-B is the main component and is preferably present in a proportion of at least 70%, in particular at least 80% and especially at least 90% of the compound I-B. Another embodiment which is also preferred relates to a racemic mixture of the enantiomer (S,S)-I-B with the enantiomer (R,R)-I-B.

A preferred embodiment of the compounds I-B are the compounds of the formula I′-B shown below:

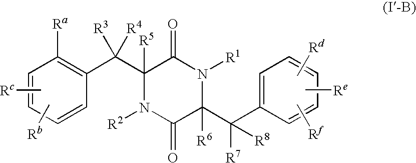

In formula I′-B, R¹, R², R³, R⁴, R⁶, R⁷, R⁸, R^{a}, R^{b}, R^{c}, R^{d}, R^{e} and R^{f} have one of the meanings mentioned above and in particular one of the meanings mentioned above or below as being preferred, where R⁵ is not together with R³ a chemical bond. From among the compounds I′-B, particular preference is given to those compounds in which R^{d} and R^{e} are hydrogen and which, with respect to the substituents R^{b}, R^{c} and R^{d}, have the substitution patter given for formula I.a. Hereinbelow, these compounds are also referred to as compounds of the formula I-B.a:

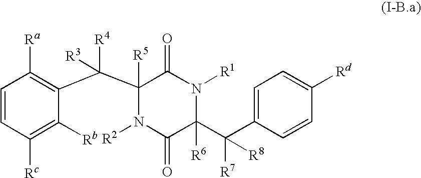

In formula I-B.a, R¹, R², R³, R⁴, R⁶, R⁷, R⁸, R^{a}, R^{b}, R^{c}, and R^{d} have preferably one of the meanings mentioned above or below as being preferred, where R⁵ is not together with R³ a chemical bond.

Preference is given in particular to the pure enantiomers of the formula (S,S)-I-B.a given below, in which R¹, R², R³, R⁴, R⁶, R⁷, R⁸, R^{a}, R^{b}, R^{c}, and R^{d} have one of the meanings mentioned above, in particular one of the meanings mentioned as being preferred or as being particularly preferred, and also to enantiomer mixtures and diastereomer mixtures of I-B.a in which the S,S enantiomer is the main component and is preferably present in a proportion of at least 70%, in particular at least 80% and especially at least 90% of the compound I-B.a. Preference is also given to the agriculturally suitable salts of the enantiomers (S,S)-I-B.a and to enantiomer mixtures and diastereomer mixtures of the salts in which the enantiomer (S,S)-I-B is the main component and is preferably present in a proportion of at least 70%, in particular at least 80% and especially at least 90% of the compound I-B.a. Another embodiment which is also preferred relates to a racemic mixture of the S,S enantiomer (S,S)-I-B.a with the R,R enantiomer (R,R)-I-B.a.

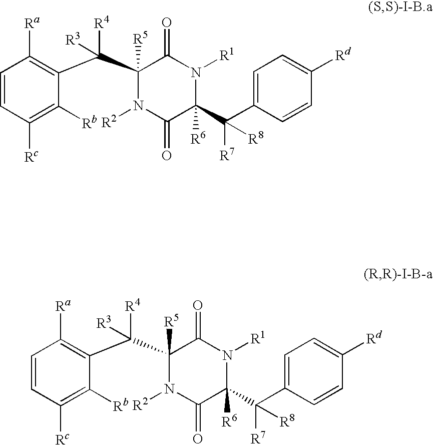

In the formula (S,S)-I-B.a or (R,R)-I-B.a, the radicals R¹, R², R³, R⁴, R⁶, R⁷, R⁸, R^{a}, R^{b}, R^{c} and R^{d} independently of one another, but preferably in combination, have in particular one of the meanings mentioned above or below as being preferred.

In the compounds of the general formula I and in the compounds of the formulae I.a, I′-A, I-A.a, I′-B and I-B.a, R⁶ is preferably halogen, cyano, nitro, C₂-C₈-alkenyl, C₂-C₈-alkynyl or C(O)R⁶¹ in which R⁶¹ has the meanings mentioned above. Preferably, R⁶¹ is C₁-C₆-alkyl or C₁-C₆-haloalkyl.

In the compounds of the general formula I and in the compounds of the formulae I.a, I′-A, I-A.a, I′-B and I-B.a, R⁷ and R⁸ are preferably hydrogen.

Preference is likewise given to compounds of the formula I or to the compounds of the formulae I.a, I′-A, I-A.a, I′-B and I-B.a in which R⁷ and R⁸ together with the carbon atom to which they are attached are a carbonyl group.

A special embodiment of the invention relates to compounds of the general formula I in which R¹ together with the radical R² is a 1-, 2-, 3- or 4-membered carbon chain in which one carbon atom may be replaced by O, S or a group NR^{A}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R¹ together with the radical R² is preferably CH₂ or CH₂CH₂. Here, the groups A¹, A², R³, R⁴, R⁵, R⁶, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R¹ together with the radical R⁵ is a 1-, 2-, 3- or 4-membered carbon chain in which one carbon atom may be replaced by O, S or a group NR^{A}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Preference is given here to compounds according to claim 24 in which R¹ together with the radical R⁵ is CH₂ or CH₂CH₂. Here, the groups A¹, A², R², R³, R⁴, R⁶, R⁷, R⁸, R^{a}, R^{b}, R^{c}, R^{e}, R^{f}, Y¹ and Y² have one of the meanings given above, in particular one of the meanings given as being preferred.

A further special embodiment of the invention relates to compounds of the general formula I in which R¹ together with a radical R^{d} attached in the ortho-position to the point of attachment of A² to a carbon atom or a nitrogen atom of A² is a covalent bond or a 1-, 2-, 3- or 4-membered carbon chain in which one carbon atom may be replaced by O, S or a group NR^{B}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R¹ together with the radical R^{d} is preferably a covalent bond, CH₂ or CH₂CH₂. Here, the groups A¹, A², R², R³, R⁴, R⁵, R⁶, R⁷, R⁸, R^{a}, R^{b}, R^{c}, R^{e}, R^{f}, Y¹ and Y² have one of the meanings given above, in particular one of the meanings given as being preferred.

A further special embodiment of the invention relates to compounds of the general formula I in which R¹ together with a radical R⁸ is a 2-, 3- or 4-membered carbon chain in which one carbon atom may be replaced by O, S or a group NR^{C}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-halo-alkoxy. Here, R¹ together with the radical R⁸ is preferably CH₂CH₂ or CH₂CH₂CH₂. Here, the groups A¹, A², R¹, R², R³, R⁴, R⁵, R⁶, R⁷, R^{a}, R^{b}, R^{c}, R^{d}, R^{e}, R^{f}, Y¹and Y² have one of the meanings given above, in particular one of the meanings given as being preferred.

A further special embodiment of the invention relates to compounds of the general formula I in which R¹ together with a radical R⁶ is a 3-, 4- or 5-membered carbon chain in which one carbon atom may be replaced by O, S or a group NR^{D}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-halo-alkoxy. Here, R¹ together with the radical R⁶ is preferably CH₂CH₂CH₂ or CH₂CH₂CH₂CH₂ in which 1, 2, 3 or 4 of the hydrogen atoms may be replaced by radicals selected from the group consisting of halogen, cyano, hydroxyl, C₁-C₄-alkyl, C₁-C₄-haloalkyl, C₁-C₄-alkoxy and C₁-C₄-haloalkoxy. Here, the groups A¹, A², R¹, R², R³, R⁴, R⁵, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R³ together with the radical R⁵ is a 1-, 2-, 3- or 4-membered carbon chain in which one carbon atom may be replaced by O, S or a group NR^{I}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R³ together with the radical R⁵ is preferably CH₂, O or a group NR^{I} in which R¹ is hydrogen or C₁-C₄-alkyl. Here, the groups A¹, A², R¹, R², R⁴, R⁶, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R³ together with the radical R⁴ is a 2-, 3-, 4- or 5-membered carbon chain in which one carbon atom may be replaced by O, S or a group NR^{K}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R³ together with the radical R⁴ is preferably CH₂CH₂, CH₂CH₂CH₂ or CH₂CH₂CH₂CH₂ in which 1, 2, 3 or 4 of the hydrogen atoms may be replaced by radicals selected from the group consisting of halogen, cyano, hydroxyl, C₁-C₄-alkyl, C₁-C₄-haloalkyl, C₁-C₄-alkoxy and C₁-C₄-haloalkoxy. Here, the groups A¹, A², R¹, R², R⁵, R⁶, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R⁴ together with the radical R^{a} is a 2-, 3-, 4- or 5-membered carbon chain in which one carbon atom may be replaced by O, S or a group NR^{L}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R⁴ together with the radical R^{a} is preferably C(O)NR^{L} or C(O)O in which R^{L} is hydrogen or C₁-C₄-alkyl. Here, the groups A¹, A², R¹, R², R³, R⁵, R⁶, R⁷, R⁸, R^{b}, R^{c}, R^{d}, R^{e}, R^{f}, Y¹ and Y² have one meanings given above, in particular one of the meanings given as being preferred.

A further special embodiment of the invention relates to compounds of the general formula I in which R⁵ together with the radical R^{a} is a 2-, 3-, 4- or 5-membered carbon chain in which one carbon atom may be replaced by O, S or a group NR^{M}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R⁵ together with the radical R^{a} is preferably CH₂CH₂ or CH₂CH₂CH₂. Here, the groups A¹, A², R¹, R², R³, R⁴, R⁶, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R⁵ together with the radical R⁶ is a 1-, 2-, 3-, 4- or 5-membered carbon chain in which one carbon atom may be replaced by O, S or a group NR^{N}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R⁵ together with the radical R⁶ is preferably CH₂ or CH₂CH₂. Here, the groups A¹, A², R¹, R², R³, R⁴, R⁷, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R⁶ together with a radical R^{d}, which is attached in the ortho-position to the point of attachment of A² to a carbon atom or a nitrogen atom of A², is a 1-, 2-, 3- or 4-membered carbon chain in which one carbon atom may be replaced by O, S or a group NR^{o}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R⁶ together with the radical R^{d} is preferably CH₂ or CH₂CH₂. Here, the groups A¹, A², R¹, R², R³, R⁴, R⁵, R⁷, R⁸, R^{a}, R^{b}, R^{c}, R^{e}, R^{f}, Y¹ and Y² have one of the meanings given above, in particular one of the meanings given as being preferred.

A further special embodiment of the invention relates to compounds of the general formula I in which R⁶ together with a radical R⁷ is a 1-, 2-, 3- or 4-membered carbon chain in which one carbon atom may be replaced by O, S or a group NR^{Q}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-halo-alkoxy. R⁶ together with the radical R⁷ is preferably CH₂, O or a group NR^{Q} in which R^{Q} is hydrogen or C₁-C₄-alkyl. Here, the groups A¹, A², R¹, R², R³, R⁴, R⁵, R⁸, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R⁷ together with the radical R⁸ is a 2-, 3-, 4- or 5-membered carbon chain in which one carbon atom may be replaced by O, S or a group NR^{R}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy. Here, R⁷ together with the radical R⁸ is preferably CH₂CH₂, CH₂CH₂CH₂ or CH₂CH₂CH₂CH₂ in which 1, 2, 3 or 4 of the hydrogen atoms may be replaced by radicals selected from the group consisting of halogen, cyano, hydroxyl, C₁-C₄-alkyl, C₁-C₄-haloalkyl, C₁-C₄-alkoxy and C₁-C₄-haloalkoxy. Here, the groups A¹, A², R¹, R², R³, R⁴, R⁵, R⁶, R^{a}, R^{b}, R^{c}, R^{d}, R^{e}, R^{f}, Y¹ and Y² have one of the meanings given above, in particular one of the meanings given as being preferred.

A further special embodiment of the invention relates to compounds of the general formula I in which R⁸ together with a radical R^{d}, which is attached in the ortho-position to the point of attachment of A² to a carbon atom or a nitrogen atom of A², is a 2-, 3-, 4- or 5-membered carbon chain in which one carbon atom may be replaced by O, S or a group NR^{S}, where one of the carbon atoms may carry a carbonyl oxygen atom and/or in which the carbon atoms, in addition to hydrogen, may carry 1, 2, 3 or 4 radicals selected from the group consisting of cyano, hydroxyl, C₁-C₄-alkyl, C₁-C₄-haloalkyl, C₁-C₄-alkoxy, C₁-C₄-haloalkoxy. Here, R⁸ together with the radical R^{d} is preferably C(O)NRS or C(O)O in which R^{S} is hydrogen or C₁-C₄-alkyl. Here, the groups A¹, A², R¹, R², R³, R⁴, R⁵, R⁶, R^{a}, R^{b}, R^{c}, R^{d}, R^{e}, R^{f}, Y¹ and Y² have one of the meanings above, in particular one of the meanings given as being preferred.

In particular with a view to their use as herbicides and active compounds for controlling unwanted vegetation, preference is given to the individual compounds compiled in Tables 1 to 88 below, are embraced by the general formulae I-A.a′ and I-B.a′ below. The groups mentioned in the individual tables for a substituent are furthermore per se, independently of the combination in which they are mentioned, a particularly preferred embodiment of the substituent in question.

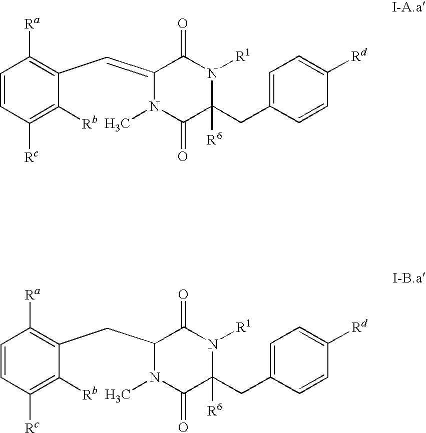

Table 1

Compounds of the formula I-A.a′ (compounds I-A.a′.1 to I-A.a′.220) in which R^{a} is CN, R^{d} is hydrogen and the combination of R^{b}, R^{c}, R¹ and R⁶ for a compound corresponds in each case to one row of Table A.

Table 2

Compounds of the formula I-A.a′ (compounds I-A.a′.221 to I-A.a′.440) in which R^{a} is CN, R^{d} is fluorine and the combination of R^{b}, R^{c}, R¹ and R⁶ for a compound corresponds in each case to one row of Table A.

Table 3

Compounds of the formula I-A.a′ (compounds I-A.a′.441 to I-A.a′.660) in which R^{a} is NO₂, R^{d} is hydrogen and the combination of R^{b}, R^{c}, R¹ and R⁶ for a compound corresponds in each case to one row of Table A.

Table 4

Compounds of the formula I-A.a′ (compounds I-A.a′.661 to I-A.a′.880) in which R^{a} is NO₂, R^{d} is fluorine and the combination of R^{b}, R^{c}, R¹ and R⁶ for a compound corresponds in each case to one row of Table A.

Table 5

Compounds of the formula I-A.a′ (compounds I-A.a′.881 to I-A.a′.1100) in which R^{a} is Br, R^{d} is hydrogen and the combination of R^{b}, R^{c}, R¹ and R⁶ for a compound corresponds in each case to one row of Table A.

Table 6

Compounds of the formula I-A.a′ (compounds I-A.a′.1101 to I-A.a′.1320) in which R^{a} is Br, R^{d} is fluorine and the combination of R^{b}, R^{c}, R¹ and R⁶ for a compound corresponds in each case to one row of Table A.

Table 7

Compounds of the formula I-A.a′ (compounds I-A.a′.1321 to I-A.a′.1540) in which R^{a} is iodine, R^{d} is hydrogen and the combination of R^{b}, R^{c}, R¹ and R⁶ for a compound corresponds in each case to one row of Table A.

Table 8

Compounds of the formula I-A.a′ (compounds I-A.a′.1541 to I-A.a′.1760) in which R^{a} is iodine, R^{d} is fluorine and the combination of R^{b}, R^{c}, R¹ and R⁶ for a compound corresponds in each case to one row of Table A.

Table 9

Compounds of the formula I-A.a′ (compounds I-A.a′.1761 to I-A.a′.1980) in which R^{a} is thiazol-2-yl, R^{d} is hydrogen and the combination of R^{b}, R^{c}, R¹ and R⁶ for a compound corresponds in each case to one row of Table A.

Table 10

Compounds of the formula I-A.a′ (compounds I-A.a′.1981 to I-A.a′.2200) in which R^{a} is thiazol-2-yl, R^{d} is fluorine and the combination of R^{b}, R^{c}, R¹ and R⁶ for a compound corresponds in each case to one row of Table A.

Table 11

Compounds of the formula I-A.a′ (compounds I-A.a′.2201 to I-A.a′.2420) in which R^{a} is thiazol-4-yl, R^{d} is hydrogen and the combination of R^{b}, R^{c}, R¹ and R⁶ for a compound corresponds in each case to one row of Table A.

Table 12

Compounds of the formula I-A.a′ (compounds I-A.a′.2421 to I-A.a′.2640) in which R^{a} is thiazol-4-yl, R^{d} is fluorine and the combination of R^{b}, R^{c}, R¹ and R⁶ for a compound corresponds in each case to one row of Table A.

Table 13

Compounds of the formula I-A.a′ (compounds I-A.a′.2641 to I-A.a′.2860) in which R^{a} is thiazol-5-yl, R^{d} is hydrogen and the combination of R^{b}, R^{c}, R¹ and R⁶ for a compound corresponds in each case to one row of Table A.

Table 14

Compounds of the formula I-A.a′ (compounds I-A.a′.2861 to I-A.a′.3080) in which R^{a} is thiazol-5-yl, R^{d} is fluorine and the combination of R^{b}, R^{c}, R¹ and R⁶ for a compound corresponds in each case to one row of Table A.

Table 15

Compounds of the formula I-A.a′ (compounds I-A.a′.3081 to I-A.a′.3300) in which R^{a} is 4-methylthiazol-2-yl, R^{d} is hydrogen and the combination of R^{b}, R^{c}, R¹ and R⁶ for a compound corresponds in each case to one row of Table A.

Table 16

Compounds of the formula I-A.a′ (compounds I-A.a′.3301 to I-A.a′.3520) in which R^{a} is 4-methylthiazol-2-yl, R^{d} is fluorine and the combination of R^{b}, R^{c}, R¹ and R⁶ for a compound corresponds in each case to one row of Table A.

Table 17

Compounds of the formula I-A.a′ (compounds I-A.a′.3521 to I-A.a′.3740) in which R^{a} is 5-methylthiazol-2-yl, R^{d} is hydrogen and the combination of R^{b}, R^{c}, R¹ and R⁶ for a compound corresponds in each case to one row of Table A.

Table 18

Compounds of the formula I-A.a′ (compounds I-A.a′.3741 to I-A.a′.3960) in which R^{a} is 5-methylthiazol-2-yl, R^{d} is fluorine and the combination of R^{b}, R^{c}, R¹ and R⁶ for a compound corresponds in each case to one row of Table A.

Table 19

Compounds of the formula I-A.a′ (compounds I-A.a′.3961 to I-A.a′.4180) in which R^{a} is oxazol-2-yl, R^{d} is hydrogen and the combination of R^{b}, R^{c}, R¹ and R⁶ for a compound corresponds in each case to one row of Table A.

Table 20

Compounds of the formula I-A.a′ (compounds I-A.a′.4181 to I-A.a′.4400) in which R^{a} is oxazol-2-yl, R^{d} is fluorine and the combination of R^{b}, R^{c}, R¹ and R⁶ for a compound corresponds in each case to one row of Table A.

Table 21

Compounds of the formula I-A.a′ (compounds I-A.a′.4401 to I-A.a′.4620) in which R^{a} is 4-methyloxazol-2-yl, R^{d} is hydrogen and the combination of R^{b}, R^{c}, R¹ and R⁶ for a compound corresponds in each case to one row of Table A.

Table 22

Compounds of the formula I-A.a′ (compounds I-A.a′.4621 to I-A.a′.4840) in which R^{a} is 4-methyloxazol-2-yl, R^{d} is fluorine and the combination of R^{b}, R^{c}, R¹ and R⁶ for a compound corresponds in each case to one row of Table A.

Table 23

Compounds of the formula I-A.a′ (compounds I-A.a′.4841 to I-A.a′.5060) in which R^{a} is 2,5-dimethyl-2H-pyrazol-3-yl, R^{d} is hydrogen and the combination of R^{b}, R^{c}, R¹ and R⁶ for a compound corresponds in each case to one row of Table A.

Table 24

Compounds of the formula I-A.a′ (compounds I-A.a′.5061 to I-A.a′.5280) in which R^{a} is 2,5-dimethyl-2H-pyrazol-3-yl, R^{d} is fluorine and the combination of R^{b}, R^{c}, R¹ and R⁶ for a compound corresponds in each case to one row of Table A.

Table 25

Compounds of the formula I-A.a′ (compounds I-A.a′.5281 to I-A.a′.5500) in which R^{a} is 1H-tetrazol-5-yl, R^{d} is hydrogen and the combination of R^{b}, R^{c}, R¹ and R⁶ for a compound corresponds in each case to one row of Table A.

Table 26

Compounds of the formula I-A.a′ (compounds I-A.a′.5501 to I-A.a′.5720) in which R^{a} is 1H-tetrazol-5-yl, R^{d} is fluorine and the combination of R^{b}, R^{c}, R¹ and R⁶ for a compound corresponds in each case to one row of Table A.

Table 27

Compounds of the formula I-A.a′ (compounds I-A.a′.5721 to I-A.a′.5940) in which R^{a} is 1-methyl-1H-tetrazol-5-yl, R^{d} is hydrogen and the combination of R^{b}, R^{c}, R¹ and R⁶ for a compound corresponds in each case to one row of Table A.

Table 28

Compounds of the formula I-A.a′ (compounds I-A.a′.5941 to I-A.a′.6160) in which R^{a} is 1-methyl-1H-tetrazol-5-yl, R^{d} is fluorine and the combination of R^{b}, R^{c}, R¹ and R⁶ for a compound corresponds in each case to one row of Table A.

Table 29

Compounds of the formula I-A.a′ (compounds I-A.a′.6161 to I-A.a′.6380) in which R^{a} is 2-methyl-2H-tetrazol-5-yl, R^{d} is hydrogen and the combination of R^{b}, R^{c}, R¹ and R⁶ for a compound corresponds in each case to one row of Table A.

Table 30

Compounds of the formula I-A.a′ (compounds I-A.a′.6381 to I-A.a′.6600) in which R^{a} is 2-methyl-2H-tetrazol-5-yl, R^{d} is fluorine and the combination of R^{b}, R^{c}, R¹ and R⁶ for a compound corresponds in each case to one row of Table A.

Table 31

Compounds of the formula I-A.a′ (compounds I-A.a′.6601 to I-A.a′.6820) in which R^{a} is 3-methyl-3H-imidazol-4-yl, R^{d} is hydrogen and the combination of R^{b}, R^{c}, R¹ and R⁶ for a compound corresponds in each case to one row of Table A.

Table 32

Compounds of the formula I-A.a′ (compounds I-A.a′.6821 to I-A.a′.7040) in which R^{a} is 3-methyl-3H-imidazol-4-yl, R^{d} is fluorine and the combination of R^{b}, R^{c}, R¹ and R⁶ for a compound corresponds in each case to one row of Table A.

Table 33

Compounds of the formula I-A.a′ (compounds I-A.a′.7041 to I-A.a′.7260) in which R^{a} is pyridin-2-yl, R^{d} is hydrogen and the combination of R^{b}, R^{c}, R¹ and R⁶ for a compound corresponds in each case to one row of Table A.

Table 34

Compounds of the formula I-A.a′ (compounds I-A.a′.7261 to I-A.a′.7480) in which R^{a} is pyridin-2-yl, R^{d} is fluorine and the combination of R^{b}, R^{c}, R¹ and R⁶ for a compound corresponds in each case to one row of Table A.

Table 35

Compounds of the formula I-A.a′ (compounds I-A.a′.7481 to I-A.a′.7700) in which R^{a} is pyridin-3-yl, R^{d} is hydrogen and the combination of R^{b}, R^{c}, R¹ and R⁶ for a compound corresponds in each case to one row of Table A.

Table 36

Compounds of the formula I-A.a′ (compounds I-A.a′.7701 to I-A.a′.7920) in which R^{a} is pyridin-3-yl, R^{d} is fluorine and the combination of R^{b}, R^{c}, R¹ and R⁶ for a compound corresponds in each case to one row of Table A.

Table 37

Compounds of the formula I-A.a′ (compounds I-A.a′7921 to I-A.a′.8140) in which R^{a} is pyridin-4-yl, R^{d} is hydrogen and the combination of R^{b}, R^{c}, R¹ and R⁶ for a compound corresponds in each case to one row of Table A.

Table 38

Compounds of the formula I-A.a′ (compounds I-A.a′.8141 to I-A.a′.8360) in which R^{a} is pyridin-4-yl, R^{d} is fluorine and the combination of R^{b}, R^{c}, R¹ and R⁶ for a compound corresponds in each case to one row of Table A.

Table 39

Compounds of the formula I-A.a′ (compounds I-A.a′.8361 to I-A.a′.8580) in which R^{a} is pyrimidin-5-yl, R^{d} is hydrogen and the combination of R^{b}, R^{c}, R¹ and R⁶ for a compound corresponds in each case to one row of Table A.

Table 40

Compounds of the formula I-A.a′ (compounds I-A.a′.8581 to I-A.a′.8800) in which R^{a} is pyrimidin-5-yl, R^{d} is fluorine and the combination of R^{b}, R^{c}, R¹ and R⁶ for a compound corresponds in each case to one row of Table A.

Table 41

Compounds of the formula I-A.a′ (compounds I-A.a′.8801 to I-A.a′.9020) in which R^{a} is pyrazin-2-yl, R^{d} is hydrogen and the combination of R^{b}, R^{c}, R¹ and R⁶ for a compound corresponds in each case to one row of Table A.

Table 42

Compounds of the formula I-A.a′ (compounds I-A.a′.9021 to I-A.a′.9240) in which R^{a} is pyrazin-2-yl, R^{d} is fluorine and the combination of R^{b}, R^{c}, R¹ and R⁶ for a compound corresponds in each case to one row of Table A.

Table 43

Compounds of the formula I-A.a′ (compounds I-A.a′.9241 to I-A.a′.9460) in which R^{a} is pyridazin-4-yl, R^{d} is hydrogen and the combination of R^{b}, R^{c}, R¹ and R⁶ for a compound corresponds in each case to one row of Table A.

Table 44

Compounds of the formula I-A.a′ (compounds I-A.a′.9461 to I-A.a′.9680) in which R^{a} is pyridazin-4-yl, R^{d} is fluorine and the combination of R^{b}, R^{c}, R¹ and R⁶ for a compound corresponds in each case to one row of Table A.

Table 45

Compounds of the formula I-B.a′ (compounds I-B.a′.1 to I-B.a′.220) in which R^{a} is CN, R^{d} is hydrogen and the combination of R^{b}, R^{c}, R¹ and R⁶ for a compound corresponds in each case to one row of Table A.

Table 46

Compounds of the formula I-B.a′ (compounds I-B.a′.221 to I-B.a′.440) in which R^{a} is CN, R^{d} is fluorine and the combination of R^{b}, R^{c}, R¹ and R⁶ for a compound corresponds in each case to one row of Table A.

Table 47

Compounds of the formula I-B.a′ (compounds I-B.a′.441 to I-B.a′.660) in which R^{a} is NO₂, R^{d} is hydrogen and the combination of R^{b}, R^{c}, R¹ and R⁶ for a compound corresponds in each case to one row of Table A.

Table 48

Compounds of the formula I-B.a′ (compounds I-B.a′.661 to I-B.a′.880) in which R^{a} is NO₂, R^{d} is fluorine and the combination of R^{b}, R^{c}, R¹ and R⁶ for a compound corresponds in each case to one row of Table A.

Table 49

Compounds of the formula I-B.a′ (compounds I-B.a′.881 to I-B.a′.1100) in which R^{a} is Br, R^{d} is hydrogen and the combination of R^{b}, R^{c}, R¹ and R⁶ for a compound corresponds in each case to one row of Table A.

Table 50

Compounds of the formula I-B.a′ (compounds I-B.a′.1101 to I-B.a′.1320) in which R^{a} is Br, R^{d} is fluorine and the combination of R^{b}, R^{c}, R¹ and R⁶ for a compound corresponds in each case to one row of Table A.

Table 51

Compounds of the formula I-B.a′ (compounds I-B.a′.1321 to I-B.a′.1540) in which R^{a} is iodine, R^{d} is hydrogen and the combination of R^{b}, R^{c}, R¹ and R⁶ for a compound corresponds in each case to one row of Table A.

Table 52

Compounds of the formula I-B.a′ (compounds I-B.a′.1541 to I-B.a′.1760) in which R^{a} is iodine, R^{d} is fluorine and the combination of R^{b}, R^{c}, R¹ and R⁶ for a compound corresponds in each case to one row of Table A.

Table 53

Compounds of the formula I-B.a′ (compounds I-B.a′.1761 to I-B.a′.1980) in which R^{a} is thiazol-2-yl, R^{d} is hydrogen and the combination of R^{b}, R^{c}, R¹ and R⁶ for a compound corresponds in each case to one row of Table A.

Table 54

Compounds of the formula I-B.a′ (compounds I-B.a′.1981 to I-B.a′.2200) in which R^{a} is thiazol-2-yl, R^{d} is fluorine and the combination of R^{b}, R^{c}, R¹ and R⁶ for a compound corresponds in each case to one row of Table A.

Table 55

Compounds of the formula I-B.a′ (compounds I-B.a′.2201 to I-B.a′.2420) in which R^{a} is thiazol-4-yl, R^{d} is hydrogen and the combination of R^{b}, R^{c}, R¹ and R⁶ for a compound corresponds in each case to one row of Table A.

Table 56

Compounds of the formula I-B.a′ (compounds I-B.a′.2421 to I-B.a′.2640) in which R^{a} is thiazol-4-yl, R^{d} is fluorine and the combination of R^{b}, R^{c}, R¹ and R⁶ for a compound corresponds in each case to one row of Table A.

Table 57

Compounds of the formula I-B.a′ (compounds I-B.a′.2641 to I-B.a′.2860) in which R^{a} is thiazol-5-yl, R^{d} is hydrogen and the combination of R^{b}, R^{c}, R¹ and R⁶ for a compound corresponds in each case to one row of Table A.

Table 58

Compounds of the formula I-B.a′ (compounds I-B.a′.2861 to I-B.a′.3080) in which R^{a} is thiazol-5-yl, R^{d} is fluorine and the combination of R^{b}, R^{c}, R¹ and R⁶ for a compound corresponds in each case to one row of Table A.

Table 59

Compounds of the formula I-B.a′ (compounds I-B.a′.3081 to I-B.a′.3300) in which R^{a} is 4-methylthiazol-2-yl, R^{d} is hydrogen and the combination of R^{b}, R^{c}, R¹ and R⁶ for a compound corresponds in each case to one row of Table A.

Table 60

Compounds of the formula I-B.a′ (compounds I-B.a′.3301 to I-B.a′.3520) in which R^{a} is 4-methylthiazol-2-yl, R^{d} is fluorine and the combination of R^{b}, R^{c}, R¹ and R⁶ for a compound corresponds in each case to one row of Table A.

Table 61

Compounds of the formula I-B.a′ (compounds I-B.a′.3521 to I-B.a′.3740) in which R^{a} is 5-methylthiazol-2-yl, R^{d} is hydrogen and the combination of R^{b}, R^{c}, R¹ and R⁶ for a compound corresponds in each case to one row of Table A.

Table 62

Compounds of the formula I-B.a′ (compounds I-B.a′.3741 to I-B.a′.3960) in which R^{a} is 5-methylthiazol-2-yl, R^{d} is fluorine and the combination of R^{b}, R^{c}, R¹ and R⁶ for a compound corresponds in each case to one row of Table A.

Table 63

Compounds of the formula I-B.a′ (compounds I-B.a′.3961 to I-B.a′.4180) in which R^{a} is oxazol-2-yl, R^{d} is hydrogen and the combination of R^{b}, R^{c}, R¹ and R⁶ for a compound corresponds in each case to one row of Table A.

Table 64

Compounds of the formula I-B.a′ (compounds I-B.a′.4181 to I-B.a′.4400) in which R^{a} is oxazol-2-yl, R^{d} is fluorine and the combination of R^{b}, R^{c}, R¹ and R⁶ for a compound corresponds in each case to one row of Table A.

Table 65

Compounds of the formula I-B.a′ (compounds I-B.a′.4401 to I-B.a′.4620) in which R^{a} is 4-methyloxazol-2-yl, R^{d} is hydrogen and the combination of R^{b}, R^{c}, R¹ and R⁶ for a compound corresponds in each case to one row of Table A.

Table 66

Compounds of the formula I-B.a′ (compounds I-B.a′.4621 to I-B.a′.4840) in which R^{a} is 4-methyloxazol-2-yl, R^{d} is fluorine and the combination of R^{b}, R^{c}, R¹ and R⁶ for a compound corresponds in each case to one row of Table A.

Table 67

Compounds of the formula I-B.a′ (compounds I-B.a′.4841 to 1-B.a′.5060) in which R^{a} is 2,5-dimethyl-2H-pyrazol-3-yl, R^{d} is hydrogen and the combination of R^{b}, R^{c}, R¹ and R⁶ for a compound corresponds in each case to one row of Table A.

Table 68

Compounds of the formula I-B.a′ (compounds I-B.a′.5061 to 1-B.a′.5280) in which R^{a} is 2,5-dimethyl-2H-pyrazol-3-yl, R^{d} is fluorine and the combination of R^{b}, R^{c}, R¹ and R⁶ for a compound corresponds in each case to one row of Table A.

Table 69

Compounds of the formula I-B.a′ (compounds I-B.a′.5281 to I-B.a′.5500) in which R^{a} is 1H-tetrazol-5-yl, R^{d} is hydrogen and the combination of R^{b}, R^{c}, R¹ and R⁶ for a compound corresponds in each case to one row of Table A.

Table 70

Compounds of the formula I-B.a′ (compounds I-B.a′.5501 to I-B.a′.5720) in which R^{a} is 1H-tetrazol-5-yl, R^{d} is fluorine and the combination of R^{b}, R^{c}, R¹ and R⁶ for a compound corresponds in each case to one row of Table A.

Table 71

Compounds of the formula I-B.a′ (compounds I-B.a′.5721 to I-B.a′.5940) in which R^{a} is 1-methyl-1H-tetrazol-5-yl, R^{d} is hydrogen and the combination of R^{b}, R^{c}, R¹ and R⁶ for a compound corresponds in each case to one row of Table A.

Table 72

Compounds of the formula I-B.a′ (compounds I-B.a′.5941 to 1-B.a′.6160) in which R^{a} is 1-methyl-1H-tetrazol-5-yl, R^{d} is fluorine and the combination of R^{b}, R^{c}, R¹ and R⁶ for a compound corresponds in each case to one row of Table A.

Table 73

Compounds of the formula I-B.a′ (compounds I-B.a′.6161 to I-B.a′.6380) in which R^{a} is 2-methyl-2H-tetrazol-5-yl, R^{d} is hydrogen and the combination of R^{b}, R^{c}, R¹ and R⁶ for a compound corresponds in each case to one row of Table A.

Table 74

Compounds of the formula I-B.a′ (compounds I-B.a′.6381 to I-B.a′.6600) in which R^{a} is 2-methyl-2H-tetrazol-5-yl, R^{d} is fluorine and the combination of R^{b}, R^{c}, R¹ and R⁶ for a compound corresponds in each case to one row of Table A.

Table 75

Compounds of the formula I-B.a′ (compounds I-B.a′.6601 to I-B.a′.6820) in which R^{a} is 3-methyl-3H-imidazol-4-yl, R^{d} is hydrogen and the combination of R^{b}, R^{c}, R¹ and R⁶ for a compound corresponds in each case to one row of Table A.

Table 76

Compounds of the formula I-B.a′ (compounds I-B.a′.6821 to I-B.a′.7040) in which R^{a} is 3-methyl-3H-imidazol-4-yl, R^{d} is fluorine and the combination of R^{b}, R^{c}, R¹ and R⁶ for a compound corresponds in each case to one row of Table A.

Table 77

Compounds of the formula I-B.a′ (compounds I-B.a′.7041 to I-B.a′.7260) in which R^{a} is pyridin-2-yl, R^{d} is hydrogen and the combination of R^{b}, R^{c}, R¹ and R⁶ for a compound corresponds in each case to one row of Table A.

Table 78

Compounds of the formula I-B.a′ (compounds I-B.a′.7261 to I-B.a′.7480) in which R^{a} is pyridin-2-yl, R^{d} is fluorine and the combination of R^{b}, R^{c}, R¹ and R⁶ for a compound corresponds in each case to one row of Table A.

Table 79

Compounds of the formula I-B.a′ (compounds I-B.a′.7481 to I-B.a′.7700) in which R^{a} is pyridin-3-yl, R^{d} is hydrogen and the combination of R^{b}, R^{c}, R¹ and R⁶ for a compound corresponds in each case to one row of Table A.

Table 80

Compounds of the formula I-B.a′ (compounds I-B.a′.7701 to I-B.a′.7920) in which R^{a} is pyridin-3-yl, R^{d} is fluorine and the combination of R^{b}, R^{c}, R¹ and R⁶ for a compound corresponds in each case to one row of Table A.

Table 81

Compounds of the formula I-B.a′ (compounds I-B.a′.7921 to I-B.a′.8140) in which R^{a} is pyridin-4-yl, R^{d} is hydrogen and the combination of R^{b}, R^{c}, R¹ and R⁶ for a compound corresponds in each case to one row of Table A.

Table 82

Compounds of the formula I-B.a′ (compounds I-B.a′.8141 to I-B.a′.8360) in which R^{a} is pyridin-4-yl, R^{d} is fluorine and the combination of R^{b}, R^{c}, R¹ and R⁶ for a compound corresponds in each case to one row of Table A.

Table 83

Compounds of the formula I-B.a′ (compounds I-B.a′.8361 to I-B.a′.8580) in which R^{a} is pyrimidin-5-yl, R^{d} is hydrogen and the combination of R^{b}, R^{c}, R¹ and R⁶ for a compound corresponds in each case to one row of Table A.

Table 84

Compounds of the formula I-B.a′ (compounds I-B.a′.8581 to I-B.a′.8800) in which R^{a} is pyrimidin-5-yl, R^{d} is fluorine and the combination of R^{b}, R^{c}, R¹ and R⁶ for a compound corresponds in each case to one row of Table A.

Table 85

Compounds of the formula I-B.a′ (compounds I-B.a′.8801 to I-B.a′.9020) in which R^{a} is pyrazin-2-yl, R^{d} is hydrogen and the combination of R^{b}, R^{c}, R¹ and R⁶ for a compound corresponds in each case to one row of Table A.

Table 86

Compounds of the formula I-B.a′ (compounds I-B.a′.9021 to I-B.a′.9240) in which R^{a} is pyrazin-2-yl, R^{d} is fluorine and the combination of R^{b}, R^{c}, R¹ and R⁶ for a compound corresponds in each case to one row of Table A.

Table 87

Compounds of the formula I-B.a′ (compounds I-B.a′.9241 to I-B.a′.9460) in which R^{a} is pyridazin-4-yl, R^{d} is hydrogen and the combination of R^{b}, R^{c}, R¹ and R⁶ for a compound corresponds in each case to one row of Table A.

Table 88

Compounds of the formula I-B.a′ (compounds I-B.a′.9461 to I-B.a′.9680) in which R^{a} is pyridazin-4-yl, R^{d} is fluorine and the combination of R^{b}, R^{c}, R¹ and R⁶ for a compound corresponds in each case to one row of Table A.

TABLE A

| | R^{b} | R^{c} | R¹ | R⁶ |
|---|---|---|---|---|
| 1. | F | H | H | F |
| 2. | F | H | H | Cl |
| 3. | F | H | H | CN |
| 4. | F | H | H | NO₂ |
| 5. | F | H | H | SCH₃ |
| 6. | F | H | H | —CH═CH₂ |
| 7. | F | H | H | —CH₂—CH═CH₂ |
| 8. | F | H | H | —CH(CH₃)—CH═CH₂ |
| 9. | F | H | H | 2-propynyl |
| 10. | F | H | H | —C(O)CH₃ |
| 11. | F | H | H | —C(O)CF₃ |
| 12. | F | H | CH₃ | F |
| 13. | F | H | CH₃ | Cl |
| 14. | F | H | CH₃ | CN |
| 15. | F | H | CH₃ | NO₂ |
| 16. | F | H | CH₃ | SCH₃ |
| 17. | F | H | CH₃ | —CH═CH₂ |
| 18. | F | H | CH₃ | —CH₂—CH═CH₂ |
| 19. | F | H | CH₃ | —CH(CH₃)—CH═CH₂ |
| 20. | F | H | CH₃ | 2-propynyl |
| 21. | F | H | CH₃ | —C(O)CH₃ |
| 22. | F | H | CH₃ | —C(O)CF₃ |
| 23. | F | F | H | F |
| 24. | F | F | H | Cl |
| 25. | F | F | H | CN |
| 26. | F | F | H | NO₂ |
| 27. | F | F | H | SCH₃ |
| 28. | F | F | H | —CH═CH₂ |
| 29. | F | F | H | —CH₂—CH═CH₂ |
| 30. | F | F | H | —CH(CH₃)—CH═CH₂ |
| 31. | F | F | H | 2-propynyl |
| 32. | F | F | H | —C(O)CH₃ |
| 33. | F | F | H | —C(O)CF₃ |
| 34. | F | F | CH₃ | F |
| 35. | F | F | CH₃ | Cl |
| 36. | F | F | CH₃ | CN |
| 37. | F | F | CH₃ | NO₂ |
| 38. | F | F | CH₃ | SCH₃ |
| 39. | F | F | CH₃ | —CH═CH₂ |
| 40. | F | F | CH₃ | —CH₂—CH═CH₂ |
| 41. | F | F | CH₃ | —CH(CH₃)—CH═CH₂ |
| 42. | F | F | CH₃ | 2-propynyl |
| 43. | F | F | CH₃ | —C(O)CH₃ |
| 44. | F | F | CH₃ | —C(O)CF₃ |
| 45. | Cl | H | H | F |
| 46. | Cl | H | H | Cl |
| 47. | Cl | H | H | CN |
| 48. | Cl | H | H | NO₂ |
| 49. | Cl | H | H | SCH₃ |
| 50. | Cl | H | H | —CH═CH₂ |
| 51. | Cl | H | H | —CH₂—CH═CH₂ |
| 52. | Cl | H | H | —CH(CH₃)—CH═CH₂ |
| 53. | Cl | H | H | 2-propynyl |
| 54. | Cl | H | H | —C(O)CH₃ |
| 55. | Cl | H | H | —C(O)CF₃ |
| 56. | Cl | H | CH₃ | F |
| 57. | Cl | H | CH₃ | Cl |
| 58. | Cl | H | CH₃ | CN |
| 59. | Cl | H | CH₃ | NO₂ |
| 60. | Cl | H | CH₃ | SCH₃ |
| 61. | Cl | H | CH₃ | —CH═CH₂ |
| 62. | Cl | H | CH₃ | —CH₂—CH═CH₂ |
| 63. | Cl | H | CH₃ | —CH(CH₃)—CH═CH₂ |
| 64. | Cl | H | CH₃ | 2-propynyl |
| 65. | Cl | H | CH₃ | —C(O)CH₃ |
| 66. | Cl | H | CH₃ | —C(O)CF₃ |
| 67. | Cl | F | H | F |
| 68. | Cl | F | H | Cl |
| 69. | Cl | F | H | CN |
| 70. | Cl | F | H | NO₂ |
| 71. | Cl | F | H | SCH₃ |
| 72. | Cl | F | H | —CH═CH₂ |
| 73. | Cl | F | H | —CH₂—CH═CH₂ |
| 74. | Cl | F | H | —CH(CH₃)—CH═CH₂ |
| 75. | Cl | F | H | 2-propynyl |
| 76. | Cl | F | H | —C(O)CH₃ |
| 77. | Cl | F | H | —C(O)CF₃ |
| 78. | Cl | F | CH₃ | F |
| 79. | Cl | F | CH₃ | Cl |
| 80. | Cl | F | CH₃ | CN |
| 81. | Cl | F | CH₃ | NO₂ |
| 82. | Cl | F | CH₃ | SCH₃ |
| 83. | Cl | F | CH₃ | —CH═CH₂ |
| 84. | Cl | F | CH₃ | —CH₂—CH═CH₂ |
| 85. | Cl | F | CH₃ | —CH(CH₃)—CH═CH₂ |
| 86. | Cl | F | CH₃ | 2-propynyl |
| 87. | Cl | F | CH₃ | —C(O)CH₃ |
| 88. | Cl | F | CH₃ | —C(O)CF₃ |
| 89. | OCH₃ | H | H | F |
| 90. | OCH₃ | H | H | Cl |
| 91. | OCH₃ | H | H | CN |
| 92. | OCH₃ | H | H | NO₂ |
| 93. | OCH₃ | H | H | SCH₃ |
| 94. | OCH₃ | H | H | —CH═CH₂ |
| 95. | OCH₃ | H | H | —CH₂—CH═CH₂ |
| 96. | OCH₃ | H | H | —CH(CH₃)—CH═CH₂ |
| 97. | OCH₃ | H | H | 2-propynyl |
| 98. | OCH₃ | H | H | —C(O)CH₃ |
| 99. | OCH₃ | H | H | —C(O)CF₃ |
| 100. | OCH₃ | H | CH₃ | F |
| 101. | OCH₃ | H | CH₃ | Cl |
| 102. | OCH₃ | H | CH₃ | CN |
| 103. | OCH₃ | H | CH₃ | NO₂ |
| 104. | OCH₃ | H | CH₃ | SCH₃ |
| 105. | OCH₃ | H | CH₃ | —CH═CH₂ |
| 106. | OCH₃ | H | CH₃ | —CH₂—CH═CH₂ |
| 107. | OCH₃ | H | CH₃ | —CH(CH₃)—CH═CH₂ |
| 108. | OCH₃ | H | CH₃ | 2-propynyl |
| 109. | OCH₃ | H | CH₃ | —C(O)CH₃ |
| 110. | OCH₃ | H | CH₃ | —C(O)CF₃ |
| 111. | OCH₃ | F | H | F |
| 112. | OCH₃ | F | H | Cl |
| 113. | OCH₃ | F | H | CN |
| 114. | OCH₃ | F | H | NO₂ |
| 115. | OCH₃ | F | H | SCH₃ |
| 116. | OCH₃ | F | H | —CH═CH₂ |
| 117. | OCH₃ | F | H | —CH₂—CH═CH₂ |
| 118. | OCH₃ | F | H | —CH(CH₃)—CH═CH₂ |
| 119. | OCH₃ | F | H | 2-propynyl |
| 120. | OCH₃ | F | H | —C(O)CH₃ |
| 121. | OCH₃ | F | H | —C(O)CF₃ |
| 122. | OCH₃ | F | CH₃ | F |
| 123. | OCH₃ | F | CH₃ | Cl |
| 124. | OCH₃ | F | CH₃ | CN |
| 125. | OCH₃ | F | CH₃ | NO₂ |
| 126. | OCH₃ | F | CH₃ | SCH₃ |
| 127. | OCH₃ | F | CH₃ | —CH═CH₂ |
| 128. | OCH₃ | F | CH₃ | —CH₂—CH═CH₂ |
| 129. | OCH₃ | F | CH₃ | —CH(CH₃)—CH═CH₂ |
| 130. | OCH₃ | F | CH₃ | 2-propynyl |
| 131. | OCH₃ | F | CH₃ | —C(O)CH₃ |
| 132. | OCH₃ | F | CH₃ | —C(O)CF₃ |
| 133. | CH₃ | H | H | F |
| 134. | CH₃ | H | H | Cl |
| 135. | CH₃ | H | H | CN |
| 136. | CH₃ | H | H | NO₂ |
| 137. | CH₃ | H | H | SCH₃ |
| 138. | CH₃ | H | H | —CH═CH₂ |
| 139. | CH₃ | H | H | —CH₂—CH═CH₂ |
| 140. | CH₃ | H | H | —CH(CH₃)—CH═CH₂ |
| 141. | CH₃ | H | H | 2-propynyl |
| 142. | CH₃ | H | H | —C(O)CH₃ |
| 143. | CH₃ | H | H | —C(O)CF₃ |
| 144. | CH₃ | H | CH₃ | F |
| 145. | CH₃ | H | CH₃ | Cl |
| 146. | CH₃ | H | CH₃ | CN |
| 147. | CH₃ | H | CH₃ | NO₂ |
| 148. | CH₃ | H | CH₃ | SCH₃ |
| 149. | CH₃ | H | CH₃ | —CH═CH₂ |
| 150. | CH₃ | H | CH₃ | —CH₂—CH═CH₂ |
| 151. | CH₃ | H | CH₃ | —CH(CH₃)—CH═CH₂ |
| 152. | CH₃ | H | CH₃ | 2-propynyl |
| 153. | CH₃ | H | CH₃ | —C(O)CH₃ |
| 154. | CH₃ | H | CH₃ | —C(O)CF₃ |
| 155. | CH₃ | F | H | F |
| 156. | CH₃ | F | H | Cl |
| 157. | CH₃ | F | H | CN |
| 158. | CH₃ | F | H | NO₂ |
| 159. | CH₃ | F | H | SCH₃ |
| 160. | CH₃ | F | H | —CH═CH₂ |
| 161. | CH₃ | F | H | —CH₂—CH═CH₂ |
| 162. | CH₃ | F | H | —CH(CH₃)—CH═CH₂ |
| 163. | CH₃ | F | H | 2-propynyl |
| 164. | CH₃ | F | H | —C(O)CH₃ |
| 165. | CH₃ | F | H | —C(O)CF₃ |
| 166. | CH₃ | F | CH₃ | F |
| 167. | CH₃ | F | CH₃ | Cl |
| 168. | CH₃ | F | CH₃ | CN |
| 169. | CH₃ | F | CH₃ | NO₂ |
| 170. | CH₃ | F | CH₃ | SCH₃ |
| 171. | CH₃ | F | CH₃ | —CH═CH₂ |
| 172. | CH₃ | F | CH₃ | —CH₂—CH═CH₂ |
| 173. | CH₃ | F | CH₃ | —CH(CH₃)—CH═CH₂ |
| 174. | CH₃ | F | CH₃ | 2-propynyl |
| 175. | CH₃ | F | CH₃ | —C(O)CH₃ |
| 176. | CH₃ | F | CH₃ | —C(O)CF₃ |
| 177. | CH═CH₂ | H | H | F |
| 178. | CH═CH₂ | H | H | Cl |
| 179. | CH═CH₂ | H | H | CN |
| 180. | CH═CH₂ | H | H | NO₂ |
| 181. | CH═CH₂ | H | H | SCH₃ |
| 182. | CH═CH₂ | H | H | —CH═CH₂ |
| 183. | CH═CH₂ | H | H | —CH₂—CH═CH₂ |
| 184. | CH═CH₂ | H | H | —CH(CH₃)—CH═CH₂ |
| 185. | CH═CH₂ | H | H | 2-propynyl |
| 186. | CH═CH₂ | H | H | —C(O)CH₃ |
| 187. | CH═CH₂ | H | H | —C(O)CF₃ |
| 188. | CH═CH₂ | H | CH₃ | F |
| 189. | CH═CH₂ | H | CH₃ | Cl |
| 190. | CH═CH₂ | H | CH₃ | CN |
| 191. | CH═CH₂ | H | CH₃ | NO₂ |
| 192. | CH═CH₂ | H | CH₃ | SCH₃ |
| 193. | CH═CH₂ | H | CH₃ | —CH═CH₂ |
| 194. | CH═CH₂ | H | CH₃ | —CH₂—CH═CH₂ |
| 195. | CH═CH₂ | H | CH₃ | —CH(CH₃)—CH═CH₂ |
| 196. | CH═CH₂ | H | CH₃ | 2-propynyl |
| 197. | CH═CH₂ | H | CH₃ | —C(O)CH₃ |
| 198. | CH═CH₂ | H | CH₃ | —C(O)CF₃ |
| 199. | CH═CH₂ | F | H | F |
| 200. | CH═CH₂ | F | H | Cl |
| 201. | CH═CH₂ | F | H | CN |
| 202. | CH═CH₂ | F | H | NO₂ |
| 203. | CH═CH₂ | F | H | SCH₃ |
| 204. | CH═CH₂ | F | H | —CH═CH₂ |
| 205. | CH═CH₂ | F | H | —CH₂—CH═CH₂ |
| 206. | CH═CH₂ | F | H | —CH(CH₃)—CH═CH₂ |
| 207. | CH═CH₂ | F | H | 2-propynyl |
| 208. | CH═CH₂ | F | H | —C(O)CH₃ |
| 209. | CH═CH₂ | F | H | —C(O)CF₃ |
| 210. | CH═CH₂ | F | CH₃ | F |
| 211. | CH═CH₂ | F | CH₃ | Cl |
| 212. | CH═CH₂ | F | CH₃ | CN |
| 213. | CH═CH₂ | F | CH₃ | NO₂ |
| 214. | CH═CH₂ | F | CH₃ | SCH₃ |
| 215. | CH═CH₂ | F | CH₃ | —CH═CH₂ |
| 216. | CH═CH₂ | F | CH₃ | —CH₂—CH═CH₂ |
| 217. | CH═CH₂ | F | CH₃ | —CH(CH₃)—CH═CH₂ |
| 218. | CH═CH₂ | F | CH₃ | 2-propynyl |
| 219. | CH═CH₂ | F | CH₃ | —C(O)CH₃ |
| 220. | CH═CH₂ | F | CH₃ | —C(O)CF₃ |

The compounds I according to the invention can be prepared by standard processes of organic chemistry. Below, some processes are illustrated by way of example.

Process A

The compounds of the formula I in which Y¹ and Y² are O can be prepared, for example, analogously to processes known from the literature by cyclizing corresponding dipeptide precursors of the formula II, for example analogously to the method described by T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965. Hereinbelow, the cyclization of dipeptides of the formula II to the compounds according to the invention is also referred to as process A and is illustrated in the scheme below.

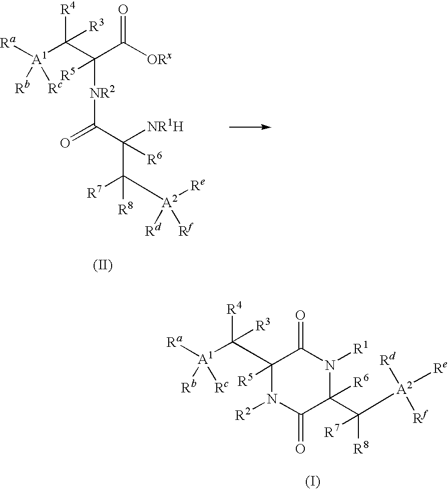

In formula II, the variables A¹, A², R¹-R⁸, R^{a}, R^{b}, R^{c}, R^{d}, R^{e} and R^{f} are as defined for formula I. The group OR^{x} is a suitable leaving group attached via oxygen. Here, R^{x} is, for example, C₁-C⁶-alkyl, in particular methyl or ethyl, or phenyl-C₁-C₆-alkyl, for example benzyl.

The cyclization can be carried out, for example, by reacting a dipeptide of the formula II either in the presence of acid or base (acidic or basic cyclization) or by heating of the reaction mixture (thermal cyclization).

The bases or acids are added to the dipeptide II either in equimolar amounts or in excess. In a particular embodiment of the process according to the invention, the bases or acids are employed in excess, based on the dipeptide.

The reaction of the dipeptide II in the presence of a base is generally carried out at temperatures in the range from 0° C. to the boiling point of the reaction mixture, preferably from 10° C. to 50° C., particularly preferably from 15° C. to 35° C. In general, the reaction is carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water and also dimethyl sulfoxide, dimethylformamide and dimethylacetamide and also morpholine and N-methylmorpholine. It is also possible to use mixtures of the solvents mentioned.

In a preferred embodiment of the invention, the reaction is carried out in a tetrahydrofuran—water mixture using, for example, a mixing ratio of 1:10 to 10:1 (parts by volume).

Suitable bases are, in general, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potasslum tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases.

In one embodiment of the process according to the invention, the reaction of II is carried out in the presence of bases, preferably in the presence of the bases potassium tert-butoxide, 2-hydroxypyridine or an aqueous solution of amonia or a mixture of these bases. Preference is given to using only one of these bases. In a particularly preferred embodiment, the reaction is carried out in an aqueous solution of ammonia which, for example, may be from 10 to 50% strength (w/v).

The reaction of II in the presence of an acid is usually carried out at temperatures in the range from 10° C. to the boiling point of the reaction mixture, preferably from 50° C. to the boiling point, particularly preferably at the boiling point under reflux. In general, the reaction is carried out in a solvent, preferably in an inert organic solvent.

In principle, suitable solvents are all those solvents which can also be used for the basic cyclization, in particular alcohols. In a preferred embodiment, the reaction is carried out in n-butanol.

In principle, suitable acids for the cyclization of II are both Brönstedt and Lewis acids. In particular, it is possible to employ inorganic acids, for example hydrohalic acids, such as hydrofluoric acid, hydrochloric acid, hydrobromic acid, inorganic oxo acids, such as sulfuric acid and perchloric acid, furthermore inorganic Lewis acids, such as boron trifluoride, aluminum trichloride, iron(III) chloride, tin(IV) chloride, titanium(IV) chloride and zinc(II) chloride, and also organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. It is, of course, also possible to use a mixture of different acids.

In one embodiment of the process according to the invention, the reaction is carried out in the presence of organic acids, for example in the presence of carboxylic acids, such as formic acid, acetic acid or trifluoroacetic acid or a mixture of these acids. Preferably, only one of these acids is used. In a preferred embodiment, the reaction is carried out in acetic acid.

A particularly preferred embodiment of the acidic cyclization is carried out in the presence of n-butanol, N-methylmorpholine and acetic acid under reflux conditions.

In a further embodiment of the invention, the reaction is carried out just by heating the reaction mixture (thermal cyclization). Here, the reaction is usually carried out at temperatures in the range from 10° C. to the boiling point of the reaction mixture, preferably from 50° C. to the boiling point of the reaction mixture, particularly preferably at the boiling point of the reaction mixture under reflux. In general, the reaction is carried out in a solvent, preferably in an inert organic solvent.

In principle, suitable solvents are those solvents which can be used for the basic cyclization. Preference is given to polar aprotic solvents, for example dimethyl sulfoxide or dimethylformamide or mixtures thereof. In a preferred embodiment, the reaction is carried out in dimethyl sulfoxide.

The reaction mixtures obtained according to one of the processes A according to the invention can, for example, be worked-up in a customary manner. This may take place, for example, by mixing with water, separating the phases and, if appropriate, chromatographic purification of the crude products. Some of the intermediates and end products are obtained in the form of viscous oils which can generally be purified or freed from volatile components under reduced pressure and at moderately elevated temperature. If the intermediates and end products are obtained as solids, the purification can also be carried out by recrystallisation or digestion.

Process B

According to a further process according to the invention (process B), the compounds of the formula I where Y¹ and Y² are O and R¹≠hydrogen can also be prepared by reacting a piperazine compound of the formula I in which R¹ is hydrogen with an alkylating agent or an acylating agent which contains the radical R¹ different from hydrogen. Such reactions can be carried out analogously to processes known from the literature, for example according to the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., J. Am. Chem. Soc. 124(47) (2002), 14017-14019, or M. Falorni et al., Europ. J. Org. Chem. (8) (2000), 1669-1675.

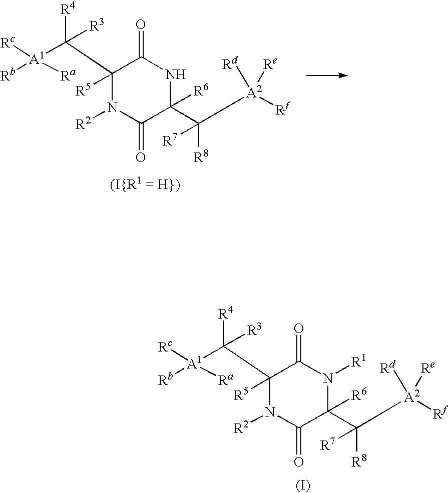

According to process B, a piperazine compound of the formula I where R¹=hydrogen is reacted with a suitable alkylating agent, hereinbelow compound X¹—R¹, or acylating agent, hereinbelow compound X²—R¹, which gives a piperazine compound of the formula I where R¹≠hydrogen.

In the alkylating agents X¹—R¹, X¹ can be halogen or O—SO₂—R^{m} where R^{m} has the meaning C₁-C₄-alkyl, C₁-C₄-alkoxy or aryl, which are optionally substituted by halogen, C₁-C₄-alkyl or halo-C₁-C₄-alkyl. In the acylating agents X²—R¹, X² may be halogen, in particular Cl. Here, R¹≠hydrogen and is as defined above.

The reaction is usually carried out at temperatures in the range from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are the compounds cited under process A, inter alia, toluene, dichloromethane, tetrahydrofuran or dimethylformamide or mixtures thereof. Preferably, the reaction is carried out in tetrahydrofuran.

In a preferred embodiment, the compound I where R¹═H is reacted with the alkylating or acylating agent in the presence of a base. Suitable bases are the compounds cited under process A. In general, the bases are employed in equimolar amounts. They can also be employed in excess or even as solvent. In a preferred embodiment of the process according to the invention, the base is added in an equimolar amount or in a substantially equimolar amount. In a further preferred embodiment, the base employed is sodium hydride.

Work-up is generally carried out analogously to the procedure described under process A.

Process C

Analogously to the procedure described under process B, it is possible to react compounds I in which Y¹ and Y² are O and R² is hydrogen with alkylating agents R²—X¹ or acylating agents R²—X², giving compounds of the formula I where R² has a meaning different from hydrogen (process C). The reaction conditions of the process C according to the invention correspond to those of process B.

Process D

The compounds of the formula I can furthermore be modified at group R^{a}. Thus, for example, compounds of the formula I in which R^{a} is CN, optionally substituted phenyl or an optionally substituted heterocyclic radical can be prepared from compounds I in which R^{a} is halogen, such as chlorine, bromine or iodine, by conversion of the substituent R^{a}, for example analogously to the methods described by J. Tsuji, Top. Organomet. Chem. (14) (2005), 332 pp., or J. Tsuji, Organic Synthesis with Palladium Compounds. (1980), 207 pp., Tetrahedron Lett. 42, 2001, S. 7473 or Org. Lett. 5, 2003, 1785.

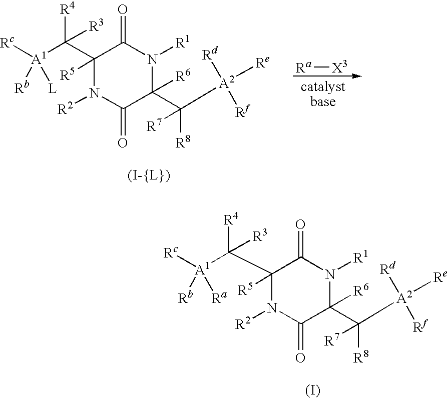

To this end, a piperazine compound of the formula I-{L} which, instead of the substituent R^{a}, has a suitable leaving group L is converted by reaction with a coupling reagent which contains a group R^{a} (compound R^{a}—X³) into another piperazine derivative of the formula I.

The reaction is usually carried out in the presence of a catalyst, preferably in the presence of a transition metal catalyst. In general, the reaction is carried out in the presence of a base.

This reaction sequence is illustrated below using the example of the substituent R^{a} and can of course be employed in an analogous manner for converting the substituents R^{b} and R^{c}.

Suitable leaving groups L are, for example, halogen, S(O)ₙR^{k} or OS(O)ₙR^{k}, where n=0, 1, 2 and R^{k} is C₁-C₆-alkyl, halo-(C₁-C₆)-alkyl or optionally halogenated or C₁-C₄-alkyl-substituted aryl.

Suitable coupling reagents X³—R^{a} are in particular those compounds in which X³, if R^{a} is C₁-C₆-alkyl, C₂-C₆-alkenyl, aryl or heteroaryl, denotes one of the following groups:
Zn—R¹ where R¹ is halogen, C₁-C₆-alkyl, C₂-C₆-alkenyl, aryl or heteroaryl;
B(OR^{m})₂ where R^{m} is H or C₁-C₆-alkyl, where two alkyl substituents together may form a C₂-C₄-alkylene chain; or
SnRⁿ₃ where Rⁿ is C₁-C₆-alkyl or aryl; and

if R^{a} is C₂-C₆-alkynyl, X³ may also be hydrogen.

Here, according to a preferred embodiment, L or R^{a} in the compounds of the formula I are attached in the ortho-position to the point of attachment of A¹ to a carbon atom of A¹.

This reaction is usually carried out at temperatures in the range from −78° C. to the boiling point of the reaction mixture, preferably from −30° C. to 65° C., particularly preferably at temperatures from 30° C. to 65° C. In general, the reaction is carried out in an inert organic solvent in the presence of a base.

Suitable solvents are the compounds cited under process A. In one embodiment of the process according to the invention, use is made of tetrahydrofuran with a catalytic amount of water; in another embodiment, only tetrahydrofuran is used.

Suitable bases are the compounds mentioned for the cyclization of the dipeptide VIII to the piperazine IV.

The bases are generally employed in equimolar amounts. They can also be employed in excess or even as solvent.

In a preferred embodiment of the process according to the invention, the base is added in an equimolar amount. In a further preferred embodiment, the base used is triethylamine or cesium carbonate, particularly preferably cesium carbonate.

Suitable catalysts for the process according to the invention are, in principle, compounds of the transition metals Ni, Fe, Pd, Pt, Zr or Cu. It is possible to use organic or inorganic compounds. Pd(PPh₃)₂Cl₂, Pd(OAc)₂, PdCl₂ or Na₂PdCl₄ may be mentioned by way of example. Here, Ph is phenyl.

The different catalysts can be employed either indicidually or else as mixtures. In a preferred embodiment of the invention, Pd(PPh₃)₂Cl₂ is used.

To prepare the compound I in which R^{a} is CN, the compound I in which L is chlorine, bromine or iodine can also be reacted with copper cyanide, analogously to known processes (see, for example, Organikum, 21. edition, 2001, Wiley, S. 404, Tetrahedron Lett. 42, 2001, S.7473 or Org. Lett. 5, 2003, 1785 and the literature cited therein).

These conversions are usually carried out at temperatures in the range of from 100° C. to the boiling point of the reaction mixture, preferably at from 100° C. to 250° C. In general, the reaction is carried out in an inert organic solvent. Suitable solvents are in particular aprotic polar solvents, for example dimethylformamide, N-methylpyrrolidone, N,N′-dimethylimidazolidin-2-one and dimethylacetamide.

Alternatively, the conversion of group R^{a} can also be carried out on the precursors of the compound I.

The work-up can be carried out analogously to the procedure described for process A.

Process E

Piperazine compounds of the formula I in which Y¹ and Y² are O and one of the groups R^{a}, R^{b} or R^{c} is COOH can furthermore be prepared from piperazine compounds of the formula I in which R^{a}, R^{b} or R^{c} is COOR^{11b}, where R^{11b} is alkyl, for example CH₃, by hydrolysis of the ester group. The hydrolysis can be performed, for example, by reaction with (H₃C)₃SnOH, for example according to K. C. Nicolaou et al., Angew. Chem. Int. Ed. Engl. (44) (2005), 1378. The carboxylic acid obtained in this manner can then be converted by standard methods of organic synthesis, if appropriate after conversion into the acid chloride, by reaction with an amine HNR^{u}R^{v} or an alcohol HOR^{w}, into the corresponding ester or the amide Organikum, Autorenkollektiv, Leipzig 1993, 19th edition, pp. 424, 429. This reaction sequence is illustrated hereinbelow using the example of the substituent R^{a}, but it is, of course, also possible to employ this sequence in an analogous manner for converting the substituents R^{b} and R^{c}.

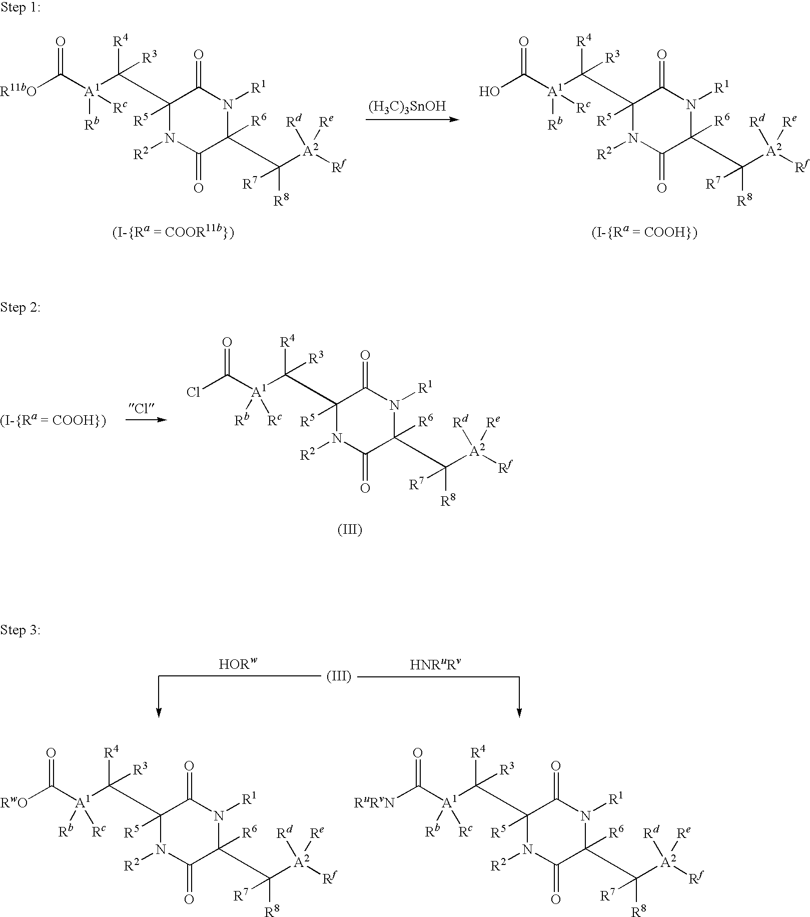

In this scheme, the variables A¹, A², R¹-R⁸, R^{b}, R^{c}, R^{d} , R^{e} and R^{f} have the meanings given above. R^{u} and R^{v} independently of one another are hydrogen, C₁-C₆-alkyl, C₂-C₆-alkenyl, C₂-C₆-alkynyl, C₁-C₆-alkoxy, C₁-C₆-alkylsulfonyl, C₁-C₆-alkylaminosulfonyl, [di-(C₁-C₆)-alkylamino]sulfonyl or optionally substituted phenyl. R^{w} is C₁-C₆-alkyl, C₃-C₆-alkenyl or C₃-C₆-alkynyl.

In a first step, the ester group in the piperazine compound I {R^{a}═COOR^{11b}} is hydrolyzed. The hydrolysis can be performed, for example, by reaction with (H₃C)₃SnOH, which gives the free acid of I {R^{a}═COOH}. The conversion into the free acid is usually carried out using an excess of (H₃C)₃SnOH. In general, the reaction is carried out in an inert organic solvent. Suitable solvents include in particular dichloroethane. In general, the reaction is carried out at elevated temperature, for example at about 80° C.

In a second step, the acid I {R^{a}═COOH} is converted into its acid chloride of the formula III. The conversion into the acid chloride is usually carried out at temperatures of from 10° C. to 50° C., preferably at room temperature, for example 25° C. In general, the reaction is carried out in an inert organic solvent. The most suitable solvents include in particular dichloromethane. In a preferred embodiment, the reaction is carried out in dichloromethane and catalytic amounts of dimethylformamide. A large number of reagents are suitable for the chlorination, for example oxalyl chloride or thionyl chloride. Preference is given to using substantially equimolar amounts of the chlorinating reagent, in particular oxalyl chloride.

The reaction with an amine NHR^{u}R^{v} in the subsequent reaction is usually carried out by adding an excess of the amine in question. The reaction can be carried out in a temperature range of from 0° C. to 40° C., preferably at room temperature, for example 25° C.

The reaction with an alcohol HOR^{w} in the subsequent reaction is usually carried out by adding an excess both of the alcohol in question and of triethylamine.

The reaction can be carried out in a temperature range of from 0° C. to 40° C., preferably at room temperature, for example 25° C.

The work-up can be carried out analogously to the procedure described for process A.

Process F

The compounds of the formula I in which Y¹ and Y² are O can be prepared according to the synthesis shown below by coupling piperazine compounds of the general formula IV with compounds V. The coupling of IV with V can be performed analogously to processes known from the literature, for example according to G. Porzi, et al., Tetrahedron 9 (19), (1998), 3411-3420, or C. I. Harding et al., Tetrahedron 60 (35), (2004), 7679-769Z or C. J. Chang et al., J. Chem. Soc. Perk. T. 1 (24), (1994), 3587-3593.

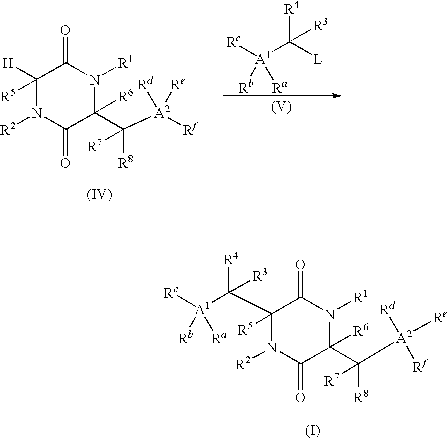

In the scheme, A¹, A², R¹-R⁸, R^{a}, R^{b}, R^{c}R_{d}, R^{e} and R^{f} are as defined above. L is a suitable leaving group, such as halogen or OSO₂R^{m}, where R^{m} is C₁-C₄-alkyl, halo-C₁-C₄-alkyl, aryl, or aryl which is mono- to trisubstituted by C₁-C₄-alkyl.

In general, the reaction is carried out at temperatures in the range from −78° C. to the boiling point of the reaction mixture, preferably in the range from −78° C. to 40° C., particularly preferably in the range from −78° C. to 30° C.

In general, the reaction is carried out in an inert organic solvent in the presence of a base. Suitable solvents are the compounds cited under process A. In a preferred embodiment of the process according to the invention, use is made of tetrahydrofuran.

Suitable bases are the compounds cited under process A. In a further preferred embodiment, the base used is lithium diisopropylamide, particularly preferably in a substantially equimolar amount, in particular in an equimolar amount.

Some compounds of the formula V are commercially available or can be prepared by transformations, described in the literature, of the corresponding commercially available precursors.

The work-up can be carried out analogously to the procedure described for process A.

Some of the precursors and intermediates required for preparing the compounds of the formula I are commercially available, known from the literature or can be prepared by processes known from the literature.

Synthesis of the Precursors

The dipeptide compounds of the formula II can be prepared, for example, from N-protected dipeptides of the general formula VI analogously to processes known from the literature, for example according to Glenn L. Stahl et al., J. Org. Chem. 43(11), (1978), 2285-6 or A. K. Ghosh et al., Org. Lett. 3(4), (2001), 635-638.

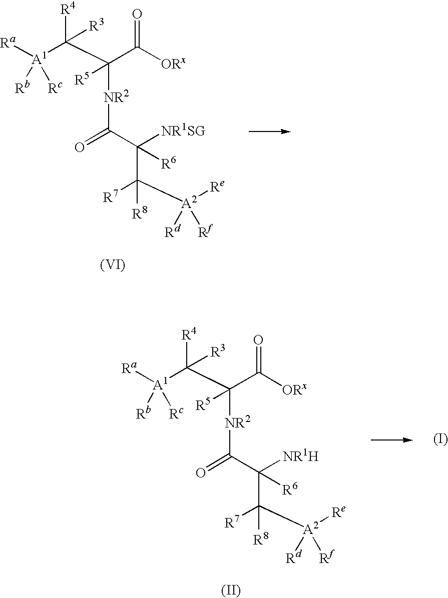

In the formulae II and VI, the variables A¹, A², R¹-R⁸, R^{a}, R^{b}, R^{c}Rd_{d}, R^{e} and R^{f} are as defined for formula I, SG is a nitrogen protective group, such as Boc (=tert-butoxycarbonyl), and ORX is a leaving group attached via an oxygen atom. Of course, in each case the preferred meanings for the compounds of the formula I apply correspondingly to the compounds of the formula II or IV. With respect to the leaving group ORX, what was stated above for the dipeptides of the formula II applies.

Thus, for example, a dipeptide of the formula VI in which SG is Boc and OR^{x} is a suitable leaving group, where R^{x} is, for example, C₁-C₆-alkyl, in particular methyl, ethyl or benzyl, can be converted in the presence of an acid into a compound of the formula II.

The reaction is usually carried out at temperatures in the range from −30° C. to the boiling point of the reaction mixture, preferably from 0° C. to 50° C., particularly preferably from 20° C. to 35° C.

The reaction can take place in a solvent, in particular in an inert organic solvent. Suitable solvents are, in principle, the compounds cited for the basic cyclization, in particular tetrahydrofuran or dichloromethane or mixtures thereof. In a preferred embodiment, the reaction is carried out in dichloromethane.

The acids used are the acids cited for process A.

In one embodiment of the process according to the invention, the reaction is carried out in the presence of organic acids, for example in the presence of strong organic acids, such as formic acid, acetic acid or trifluoroacetic acid or mixtures thereof. In a preferred embodiment, the reaction is carried out in the presence of trifluoroacetic acid.

The work-up can be carried out analogously to the procedure described for process A.

The protected dipeptides of the formula VI can be prepared analogously to processes known from the literature, for example according to Wilford L. Mendelson et al., Int. J. Peptide & Protein Research 35(3), (1990), 249-57. A typical route is the amidation of a Boc-protected amino acid VIII with an amino acid ester of the formula VII, as shown in the scheme below:

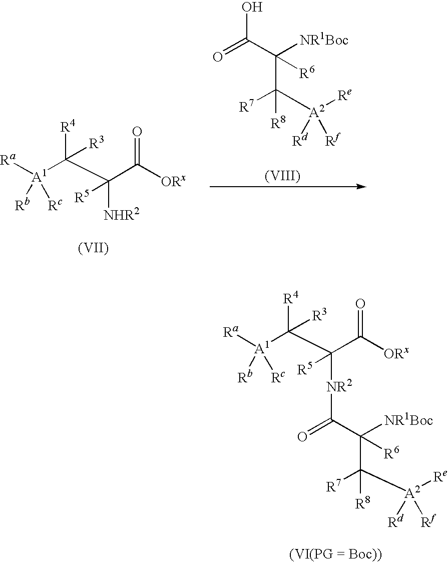

In this scheme, the variables are as defined above. Instead of Boc, it is also possible to use other amino protective groups.

In general, the reaction of VII with VIII is carried out at temperatures in a range from −30° C. to the boiling point of the reaction mixture, preferably from 0° C. to 50° C., particularly preferably from 20° C. to 35° C. The reaction can be carried out in a solvent, preferably in an inert organic solvent. Suitable solvents are the solvents mentioned for process A in connection with the basic cyclization.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, 1-ethyl-3-(dimethylaminopropyl)carbodiimide (EDAC), carbonyldiimidazole, chlorocarbonic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride. A further suitable activating agent is O-(7-azabenzotriazole-1-yl)-N,N,N′,N′-tetramethyluronium hexafluorophosphate (HATU). According to one embodiment, a preferred activating agent is EDAC or DCC.

The reaction of compounds of the formulae VII and VIII is preferably carried out in the presence of a base. Suitable bases are the compounds cited under process A. In one embodiment, the base used is triethylamine or N-ethyldiisopropylamine or mixtures thereof, particularly preferably N-ethyldiisopropylamine.

The work-up can be carried out analogously to the procedure described for process A.

For their part, the compounds of the formula VII can be prepared by deprotecting corresponding protected amino acid compounds IX analogously to processes known from the literature, for example according to Glenn L. Stahl et al., J. Org. Chem. 43(11), (1978), 2285-6 or A. K. Ghosh et al., Org. Lett. 3(4), (2001), 635-638. The preparation of VII from a Boc-protected amino acid compound IX is shown in the scheme below. Instead of the Boc group, it is also possible to use other amino protective groups

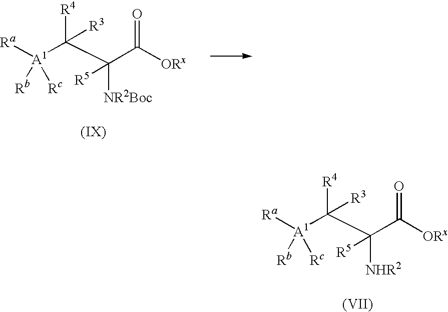

The conversion of a compound of the formula IX into the compound VII is typically carried out in the presence of an acid at temperatures in a range from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the compounds mentioned under the basic cyclization, in particular tetrahydrofuran or dichloromethane or mixtures thereof. In a preferred embodiment, the reaction is carried out in dichloromethane.

The acids and acidic catalysts used are the compounds cited for process A.

In one embodiment of the process according to the invention, the reaction is carried out in the presence of organic acids, for example in the presence of strong organic acids, such as formic acid, acetic acid or trifluoroacetic acid or mixtures thereof. In a preferred embodiment, the reaction is carried out in the presence of trifluoroacetic acid.

The work-up can be carried out analogously to the procedure described for process A.

The compounds of the formula IX can be prepared according to the reaction shown in the scheme below. The reaction of compound V with the protected amino acid compound X can be carried out analogously to processes known from the literature, for example according to I. Ojima et al., J. Am. Chem. Soc., 109(21), (1987), 6537-6538 or J. M. McIntosh et al., Tetrahedron 48(30), (1992), 6219-6224.

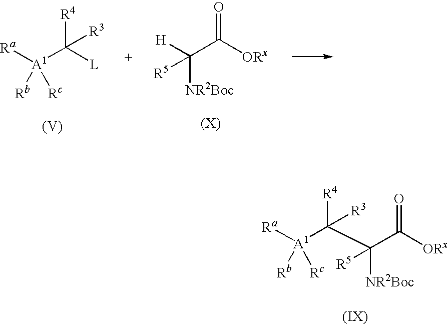

In this scheme, the variables are as defined above. L is a leaving group, for example one of the leaving groups mentioned for process F. Instead of Boc, it is also possible to use other amino protective groups.

The reaction of V with X is generally carried out in the presence of base. Suitable bases are the compounds cited under process A. In a further preferred embodiment, the base used is lithium diisopropylamide, particularly preferably in a substantially equimolar amount, in particular in an equimolar amount.

Usually, the reaction is carried out at temperatures in the range from −78° C. to the boiling point of the reaction mixture, preferably from −78° C. to the boiling point, particularly preferably from −78° C. to 30° C.

The reaction can be carried out in a solvent, preferably in an inert organic solvent. Suitable solvents are, in principle, the solvents mentioned under the basic cyclization, in particular dichloromethane or tetrahydrofuran or mixtures thereof. In a preferred embodiment, the reaction is carried out in tetrahydrofuran.

The work-up can be carried out analogously to the procedure described for process A.

Some of the compounds of the formula V are commercially available or can be prepared by transformations, described in the literature, of the corresponding commercially available precursors.

Some of the amino acid derivates of the formula VIII or X or the derivative XV described below are likewise commercially available or can be prepared by transformations, described in the literature, of the corresponding commercially available precursors.

The compounds of the formula IV where R¹ has a meaning different from hydrogen can be prepared by reacting a piperazine compound of the formula IV in which R¹ is hydrogen with an alkylating agent or acylating agent which contains the radical R¹ different from hydrogen. In an analogous manner, it is possible to prepare compounds IV where R²≠hydrogen by reacting a piperazine compound of the formula IV in which R² is hydrogen with an alkylating agent or acylating agent which contains the radical R² different from hydrogen. Such reactions can be carried out analogusly to processes known from the literature, for example according to the methods described by I. O. Donkor et al., Bioorg. Med. Chem. Lett. 11 (19) (2001), 2647-2649, B. B. Snider et al., Tetrahedron 57 (16) (2001), 3301-3307, I. Yasuhiro et al., J. Am. Chem. Soc. 124(47) (2002), 14017-14019, or M. Falorni et al., Europ. J. Org. Chem. (8) (2000), 1669-1675.

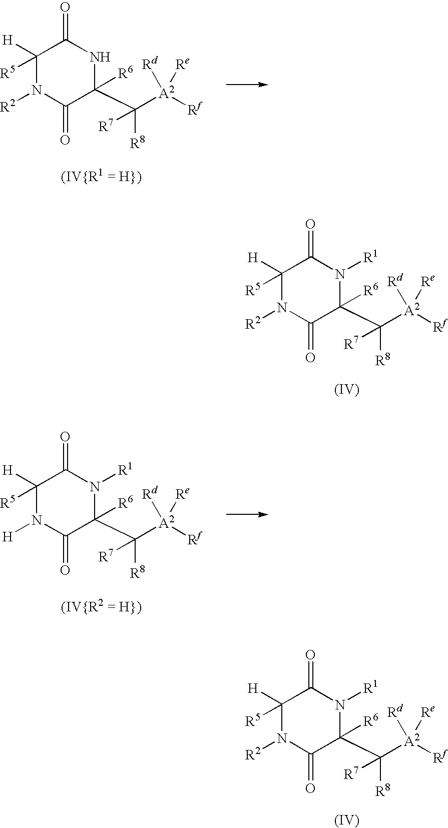

With respect to the alkylating agent or a cycling agent, what was stated for the processes B and C applies in the same manner. With respect to the reaction conditions of these reactions, what was stated for the processes B and C likewise applies. The compounds of the formula IV can also be prepared by intramolecular cyclization of compounds of the general formula XIII analogously to further processes known from the literature, for example according to T. Kawasaki et al., Org. Lett. 2(19) (2000), 3027-3029.

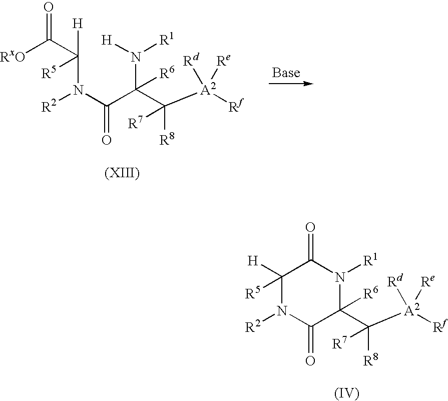

In formula XIII, the variables R^{x}, A², R¹, R², R⁵, R⁶, R⁷, R⁸, R^{d}, R^{e} and R^{f} are as defined above. The group OR^{x} is a suitable leaving group attached via oxygen. Here, R^{x} is, for example, C₁-C₆-alkyl, in particular methyl or ethyl, or phenyl-C₁-C₆-alkyl, for example benzyl.

The cyclization of the compounds of the formula XIII can be carried out in the presence of a base. In this case, the reaction is generally carried out at temperatures in the range from 0° C. to the boiling point of the reaction mixture, preferably from 10° C. to 50° C., particularly preferably from 15° C. to 35° C. The reaction can be carried out in a solvent, preferably in an inert organic solvent.

Suitable solvents are, in principle, the compounds cited under the thermal cyclization, in particular a tetrahydrofuran-water mixture having a mixing ratio of from 1:10 to 10:1.

Suitable bases are the bases mentioned for the basic cyclization according to process A, in particular potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which, for example, may be from 10 to 50% strength (w/v).

For their part, the compounds of the formula XIII can be prepared by the synthesis illustrated in the scheme below, analogously to processes known from the literature, for example according to Wilford L. Mendelson et al., Int. J. Peptide & Protein Research 35(3), (1990), 249-57, Glenn L. Stahl et al., J. Org. Chem. 43(11), (1978), 2285-6 or A. K. Ghosh et al., Org. Lett. 3(4), (2001), 635-638.

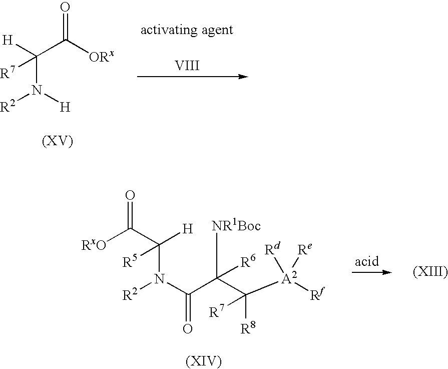

In the scheme, the variables R^{x}, A², R¹, R², R⁵, R⁶, R⁷, R⁸, R^{d}, R^{e} and R^{f} are as defined above. In a first step, the synthesis comprises the coupling of amino acid compounds XV with Boc-protected amino acids VIII in the presence of an activating agent.

The reaction of a compound of the formula XV with a compound of the formula VIII is usually carried out at temperatures in the range from −30° C. to the boiling point of the reaction mixture, preferably from 0° C. to 50° C., particularly preferably from 20° C. to 35° C. The reaction can be carried out in a solvent, preferably in an inert organic solvent. For further details, reference is made to the preparation of compound VI by amidation of the amino acid compound VIII with the compound VII.

In general, the reaction requires the presence of an activating agent. Suitable activating agents are condensing agents, such as, for example, polystyrene- or non-polystyrene-supported dicyclohexylcarbodiimide (DCC), diisopropylcarbodiimide, 1-ethyl-3-(dimethylaminopropyl)carbodiimide (EDAC), carbonyldiimidazole, chlorocarbonic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCI) or sulfonyl chlorides, such as methanesulfonyl chloride, toluenesulfonyl chloride or benzenesulfonyl chloride. A further suitable activating agent is O-(7-azabenzotriazole-1-yl)-N,N,N′,N′-tetramethyluronium hexafluorophosphate (HATU). According to one embodiment, a preferred activating agent is EDAC or DCC.

The reaction of XV with VIII is preferably carried out in the presence of a base. Suitable bases are the compounds cited under process A. In one embodiment, the base used is triethylamine or N-ethyldiisopropylamine or mixtures thereof, particularly preferably N-ethyldiisopropylamine.

The work-up can be carried out analogously to the procedure described for process A.

The deprotection of the compound XIV to give the compound XIII is typically carried out by treatment with an acid. The reaction is usually carried out at temperatures in the range from −30° C. to the boiling point of the reaction mixture, preferably from 0° C. to 50° C., particularly preferably from 20° C. to 35° C. The reaction can be carried out in a solvent, preferably in an inert organic solvent.

Suitable solvents are, in principle, the solvents mentioned under process A in connection with the basic cyclization, in particular tetrahydrofuran or dichloromethane or mixtures thereof. In a preferred embodiment, the reaction is carried out in dichloromethane.

The acids used are the acids mentioned for process A. For further details, reference is also made to the deprotection of VI to give compound II. The reaction conditions mentioned there are also suitable for deprotecting compound XIV. In one embodiment of the process according to the invention, the reaction is carried out in the presence of organic acids, in particular strong organic acids, for example in the presence of formic acid, acetic acid or trifluoroacetic acid or mixtures thereof. In a preferred embodiment, the reaction is carried out in the presence of trifluoroacetic acid.

Process F

The compounds of the formula I according to the invention can also be provided, for example, from corresponding precursor compounds in which R⁶ is hydrogen and in which R³ together with R⁵ is preferably a chemical bond. The radical R⁶ can be introduced by customary methods of organic chemistry which depend on the nature of the radical R⁶ in question, for example by alkylation, acylation, nitration, reaction with phosphorus halogen compounds, halogenation, cyanation, thionylation or sulfonylation.

For this purpose, the precursor, i.e. a compound of the formula I in which R⁶ is, instead of the given meanings, hydrogen, is deprotonated by reaction with a base in a position adjacent to the C(═Y²)N carbon, and the anion obtained in this manner is reacted with a compound X⁶—R^{6a}. Here, X⁶ is a leaving group. R^{6a} has one of the meanings given for R⁶ or is a protected precursor of the group R⁶.

Suitable bases for deprotonating the precursor are inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide, potassium amide and alkali metal silazanes, such as lithium hexamethyldisilazane or potassium hexamethyldisilazane, alkali metal and alkaline earth metal carbonates, such as lithium carbonate, potassium carbonate, cesium carbonate and calcium carbonate, and also alkali metal bicarbonate, such as sodium bicarbonate, organometallic compounds, in particular alkali metal alkyls and alkali metal aryls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. The bases are generally employed in equimolar amounts. They can also be employed in excess or even as solvent. In a preferred embodiment, the base is employed in an equimolar amount or in an essentially equimolar amount. Preferably, the base used is an alkali metal hydride, alkali metal amide or alkali metal silazane.

After the deprotonation, the precursor is reacted with a suitable compound of the formula X⁶—R^{6a}, which gives a piperazine compound of the formula I according to the invention. In the compounds X⁶—R^{6a}, X⁶ is in particular halogen, especially chlorine, bromine or iodine, a group O—C(O)R^{m} or a group O—SO₂—R^{m} where R^{m} is C₁-C₄-alkyl or aryl, which are optionally substituted by halogen, C₁-C₄-alkyl or halo-C₁-C₄-alkyl.

The reaction is usually carried out at temperatures in the range of from −78° C. to the boiling point of the reaction mixture, preferably from −50° C. to 65° C., particularly preferably from −30° C. to 65° C. In general, the reaction is carried out in a solvent, preferably in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, dimethyl sulfoxide, N-methylpyrrolidone, dimethylformamide and dimethylacetamide, and also morpholine and N-methylmorpholine and mixtures thereof. Preferred solvents are toluene, dichloromethane, tetrahydrofuran, N-methylpyrrolidone or dimethylformamide and mixtures thereof.

In addition, to prepare compounds I in which R⁶ is halogen, in particular chlorine or bromine, the precursor in which R⁶ is hydrogen can be converted in the manner described above into its anion and then be reacted with a halogenating agent, such as tetrachlorodibromoethane, N-bromosuccinimide or N-chlorosuccinimide.

In addition, to prepare compounds I in which R⁶ is cyano, the precursor in which R⁶ is hydrogen can be converted in the manner described above into its anion and then be reacted with cyanogen bromide. Alternatively, the precursor in which R⁶ is hydrogen can initially be oxidized with an organic peroxide or hydroperoxide, such as tert-butyl hydroperoxide, in the presence of transition metal catalysts, for example ruthenium compounds, such as RuCl₂(P(C₆H₅)₃)₃. The cyano group can be introduced be subsequent reaction of the oxidation product with trimethylsilyl cyanide in the presence of titanium tetrachloride (see J. Am. Chem. Soc. 112 (21), 1990, pp. 7820-7822).

The preparation of compounds I in which R⁶ is nitro can be carried out, for example, by reacting the precursor in which R⁶ is hydrogen with sodium nitrite in the presence of acetic acid analogously to the procedure given in Arch. Pharm. 326 (11), 1993, pp. 875-878.

The preparation of compounds I in which R⁶ is a radical OR⁶⁵ can be carried out, for example, by converting the precursor in which R⁶ is hydrogen into its anion in the manner described above, followed by an oxidation with phenylseleninum bromide, preferably in the presence of DMAP, according to the procedure described in J. Org. Chem. 65(15), 2000, pp. 4685-4693, which gives a compound of the formula I in which R⁶ is OH. To introduce the radical R⁶⁵, the OH group can then be alkylated or arylated by standard processes.

Hereinbelow, the corresponding precursors in which R³ and R⁵ together are a chemical bond are referred to as compounds of the formula XVI:

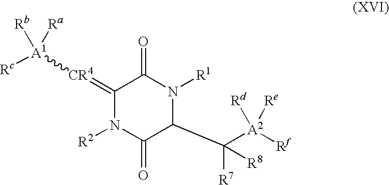

Here, A¹, A², R¹, R², R⁴, R⁷, R⁸ and R^{a} to R^{f} have one of the meanings given above. Additionally, R¹ and R² in formula XVI may be a protective group or hydrogen. With respect to the protective groups, what was said above for the compounds of the formula XIV applies.

If R¹ and/or R² in formula XVI are/is a protective group, the protective group will be removed. In this manner, a compound XVI is obtained in which R¹ and, if appropriate, R² is/are hydrogen.

The compound XVI in which R¹ is hydrogen is then reacted with an alkylating agent of the formula R¹—X¹ or an acylating agent of the formula R¹—X², preferably in the presence of a base. If R² is hydrogen, the compound XVI is reacted with an alkylating agent of the formula R²—X¹ or an acylating agent of the formula R²—X², preferably in the presence of a base.

Compounds of the formula XVI are known, for example from PCT/EP2007/050067 (=WO 2007/077247), the entire content of which is hereby included be way of reference.

The preparation of the compounds XVI is generally carried out by dehydrating the corresponding alcohol XVIa,

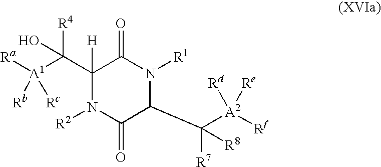

In formula XVIa, A¹, A², R¹, R², R⁴, R⁷, R⁸ and R^{a} to R^{f} have the meanings mentioned above, in particular one of the meanings mentioned as being preferred. In a first variant (variant F.1), the alcohol function of the compound XVIa can initially be converted into a suitable leaving group, and this can then be eliminated formally as compound H-LG. The elimination reaction is preferably carried out in the presence of a suitable base.

The leaving group LG is a customary leaving group easy to prepare from a hydroxyl group. Examples of these are 4-toluenesulfonyloxy (LG=-O—SO₂C₆H₄CH₃), trifluoromethanesulfonyloxy (LG=-O—SO₂CF₃) and methanesulfonyloxy (LG=-O—SO₂CH₃), the latter being particularly suitable. Such a leaving group is introduced according to customary processes, for example by reacting the alcohol XVIa with a base and then with the appropriate sulfonyl chloride, for example with methanesulfonyl chloride or trifluoromethanesulfonyl chloride. Suitable bases are the bases listed below for the elimination. However, preference is given to using bases which are soluble in organic solvents, for example the amines or nitrogen heterocycles mentioned below. In particular, use is made of pyridine or substituted pyridines, such as dimethylaminopyridine, lutidine or collidine, or mixtures thereof. Expediently, the organic bases are chosen such that they also act as solvent.

Bases suitable for the elimination are, in general inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases.

Particularly suitable are, however, bases which are sufficiently basic, but essentially not nucleophilic, for example sterically hindered alkali metal alkoxides, for example alkali metal tert-butoxides, such as potassium tert-butoxide, and in particular cyclic amidines, such as DBU (1,8-diazabicyclo[5.4.0]undec-7-ene) and DBN (1,5-diazabicyclo[3.4.0]-non-5-ene). Preference is given to using the amidines mentioned last.

The elimination is generally carried out in a solvent, preferably in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, tert-butanol, water, and also dimethyl sulfoxide, dimethylformamide and dimethylacetamide, and also morpholine and N-methylmorpholine. It is also possible to use mixtures of the solvents mentioned. Preference is given to using tetrahydrofuran.

The dehydration of alcohols XVIa by conversion of the alcohol function into a good leaving group and subsequent elimination can be carried out analogously to known processes of the prior art, for example analogously to the processes described in Helv. Chim. Acta 1947, 30, 1454; Liebigs Ann. Chem 1992, (7), 687-692, Carbanions. 24. Rearrangements of (E)- and (Z)-2,2-diphenyl-3-pentenylalkali metal compounds; Sch. Chem., Georgia Inst. Technol., Atlanta, Ga., USA; J. Org. Chem. 1989, 54(7), 1671-1679; Chemical & Pharmaceutical Bulletin 1986, 34(7), 2786-2798, the entire contents of which are included herein by way of reference.

In a second variant (variant F.2), the preparation of the compound XVI by dehydration of the compound XVIa is carried out in the presence of a suitable dehydrating agent.

Suitable dehydrating agents are, for example, the system triphenylphosphine/DEAD (DEAD=diethyl azodicarboxylate) and Burgess reagent. In general, the combination of triphenylphosphine and DEAD is employed for the targeted inversion at a hydroxyl-substituted center of chirality (Mitsunobu reaction); however, in the presence of nucleophiles it acts as a milde dehydrating agent. With respect to the compound XVIa, the system is preferably employed in excess, where the two components triphenylphosphine and DEAD are suitable present in an approximately equimolar ratio.

Burgess reagent is the zwitterion methyl N-(triethylammoniumsulfonylcarbamate ((C₂H₅)₃N⁺—SO₂—N⁻—COOCH₃), a mild dehydrating agent. With respect to the alcohol XVI, this can be employed in equimolar amounts or in a molar excess. The reaction with Burgess reagent is usually carried out in an inert organic solvent. Suitable inert organic solvents include aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, and ketones, such as acetone, methyl ethyl ketone, diethyl ketone and tert-butyl methyl ketone. Preference is given to using aromatic hydrocarbons or mixtures thereof and especially toluene.

The dehydration of alcohols XVIa with dehydration agents can be carried out analogously to known processes of the prior art, for example analogously to the processes described in Synthesis 2003, 201 and J. Indian Sci. 2001, 81, 461, the entire contents of which are included herein by way of reference.

The alcohols of the formula XVIa can be prepared, for example, analogously to processes known from the literature by cyclization of corresponding dipeptide precursors, for example analogously to the method described by T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

The alcohols of the formula XVIa in which R⁴ is hydrogen can also be prepared by coupling, in an aldol reaction, a benzaldehyde of the formula XV with a piperazin compound XVII, as illustrated in the scheme below:

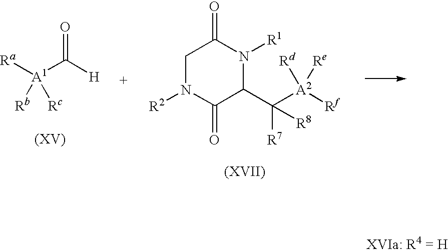

In the formulae XV and XVII, the variables A¹, A², R¹, R², R⁷, R⁸ and R^{a} to R^{f} have one of the meanings given above.

The reaction of XV with XVII in the sense of an aldol reaction is generally carried out in the presence of suitable bases. Suitable bases are those which are usually employed for aldol reactions. Suitable reaction conditions are known from the prior art and are described, for example, in J. Org. Chem. 2000, 65 (24), 8402-8405, the entire content of which is hereby included by way of reference.

The reaction of the compound XV with the compound XVII can also afford the corresponding aldol condensation product, i.e. compounds of the formula XVI, directly. This is the case in particular when in the compound XVII the radicals R¹ and R² are acyl groups, for example a group of the formula C(O)R²¹— in which R²¹ has one of the meanings given above and is in particular C₁-C₄-alkyl, for example methyl.

Such aldol condensations can be carried out analogously to the processes described in J. Org. Chem. 2000, 65 (24), 8402-8405, Synlett 2006, 677 and J. Heterocycl. Chem. 1988, 25, 591, the entire contents of which are hereby included by way of reference.

The aldol condensation is typically carried out in the presence of suitable bases. Suitable bases are those which are usually employed for aldol condensations. Preference is given to using an alkali metal or alkaline earth metal carbonate as base, for example sodium carbonat, potassium carbonat or cesium carbonat or mixtures thereof.

The reaction is preferably carried out in an inert, preferably aprotic organic solvent. Examples of suitable solvents are in particular dichloromethane, dichloroethane, chlorbenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide. Preferred solvents are in particular selected from the group consisitng of dimethylformamide, N-methylpyrrolidone and dimethylacetamide.

The temperatures required for the aldol condensation are generally in the range of from 0° C. to the boiling point of the solvent used and in particular in the range of from 10 to 80° C.

For the reaction of XV with XVII, it has been found to be advantageous for the radicals R¹ and R² in the compound XVII to be acyl groups, for example a group of the formula C(O)R²¹. The introduction of these protective groups into the compound XVII can be carried out analogously to known processes of protective group chemistry, for example by reacting the corresponding NH-free compound (compound of the formula XVII where R¹, R²═H) with anhydrides of the formula (R²¹C(O))₂O, for example according to the method described by Green, Wuts, Protective Groups in Organic Synthesis, 3rd ed. 1999, John Wiley and Sons, p. 553. The removal of a protective group R¹, R² can be carried out analogously to known processes of protective group chemistry.

If the radicals R¹ and R² in the compound XVII are acyl groups, these radicals will preferably be removed after the aldol condensation, which gives a compound of the formula XVI where R¹═R²=hydrogen. The radicals R¹ and R² are generally removed by hydrolysis, the radical R² frequently already being cleaved off under the conditions of an aldol condensation. Into the resulting compound XVI where R¹═R²=hydrogen, the radical R¹ and, if appropriate, the radical R² are then introduced, for example, by N-alkylation.

Analogously to the method described above, it is also possible to provide compounds of the formula I-A in which R⁴ is hydrogen and R³ together with R⁵ is a chemical bond and R⁶ has a meaning different from hydrogen.

The compounds of the formula XVII can be prepared by intramolecular cyclization of compounds of the general formula XVIII analogously to other processes known from the literature, for example according to T. Kawasaki et al., Org. Lett. 2(19) (2000), 3027-3029, Igor L. Rodionov et al., Tetrahedron 58(42) (2002), 8515-8523 or A. L. Johnson et al., Tetrahedron 60 (2004), 961-965.

If appropriate, the cyclization is followed by the introduction of a group R¹ or R² different from hydrogen if R¹ and/or R² in the formula XVII is hydrogen.

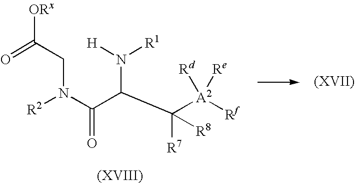

In formula XVIII, the variables A², R¹, R², R⁷, R⁸ and R^{d} to R^{f} have the meanings mentioned above. Here, R^{x} is, for example, C₁-C₆-alkyl, in particular methyl or ethyl, or phenyl-C₁-C₆-alkyl, for example benzyl.

The cyclization of the compounds of the formula XVII can be carried out in the presence of a base. In this case, the reaction is generally carried out at temperatures in the range of from 0° C. to the boiling point of the reaction mixture, preferably from 10° C. to 50° C., particularlt preferably from 15° C. to 35° C. The reaction can be carried out in a solvent, preferably in an inert organic solvent.

Suitable inert organic solvents include aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as dichloromethane, dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol, 2-butanol, isobutanol, tert-butanol, water, and also dimethyl sulfoxide, dimethylformamide and dimethylacetamide, and also morpholine and N-methylmorpholine. It is also possible to use mixtures of the solvents mentioned. The preferred solvent is a tetrahydrofuran/water mixture having a mixing ratio of from 1:10 to 10:1.

Suitable bases are, for example, inorganic compounds, such as alkali metal and alkaline earth metal hydroxides, such as lithium hydroxide, sodium hydroxide, potassium hydroxide or calcium hydroxide, an aqueous solution of ammonia, alkali metal or alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, for example lithium diisopropylamide, sodium amide and potassium amide, alkali metal and alkaline earth metal carbonates, such as lithium carbonate, potassium carbonate, cesium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example tertiary amines, such as trimethylamine, triethylamine, diisopropylethylamine, 2-hydroxypyridine and N-methylpiperidine, pyridine, substituted pyridines, such as collidine, lutidine and 4-dimethylaminopyridine, and also bicyclic amines. It is, of course, also possible to use a mixture of different bases. Preference is given in particular to potassium tert-butoxide, 2-hydroxypyridine or an aqueous solution of ammonia or a mixture of these bases. Preferably, only one of these bases is used. In a particularly preferred embodiment, the reaction is carried out in the presence of an aqueous solution of ammonia which may, for example, be of a strength of from 10 to 50% w/v. In another particularly preferred embodiment, the cyclization is carried out in a mixture comprising butanol, for example n-butanol, 2-butanol and/or isobutanol or a mixture thereof, and N-methylmorpholine, preferably under reflux conditions.

The cyclization of XVIII to XVII can also be carried out with acid catalysis, in the presence of activating compounds or thermally. The reaction of XVIII in the presence of an acid is usually carried out at temperatures in the range of from 10° C. to the boiling pint of the reaction mixture, preferablt from 50° C. to the boiling point, particularly preferably at the boiling point under reflux. In general, the reaction is carried out in a solvent, preferably in an inert organic solvent.

Suitable solvents are, in principle, those which can also be used for the basic cyclization, in particular alcohols. In a preferred embodiment, the reaction is carried out in n-butanol or a mixture of different butanol isomers (for example a mixture of n-butanol and 2-butanol and/or isobutanol).

Suitable acids for the cyclization of XVIII to XVII are, in principle, both Brönstedt and Lewis acids. Use may be made in particular of inorganic acids, for example hydrohalic acids, such as hydrofluoric acid, hydrochloric acid, hydrobromic acid, inorganic oxoacids, such as sulfuric acid and perchloric acid, furthermore of inorganic Lewis acids, such as borin trifluoride, aluminum trichloride, iron(III) chloride, tin(IV) chloride, titanium(IV) chloride and zinc(II) chloride, and also of organic acids, for example carboxylic acids and hydroxycarboxylic acids, such as formic acid, acetic acid, propionic acid, oxalic acid, citric acid and trifluoroacetic acid, and also organic sulfonic acids, such as toluenesulfonic acid, benzenesulfonic acid, camphorsulfonic acid and the like. Of course, it is also possible to use a mixture of different acids.

In one embodiment of the process according to the invention, the reaction is carried out in the presence of organic acids, for example in the presence of carboxylic acids, such as formic acid, acetic acid or trifluoroacetic acid or a mixture of these acids. Preferably, only one of these acids is used. In a preferred embodiment, the reaction is carried out in acetic acid.

In a particularly preferred embodiment, the acidic cyclization is carried out in a mixture comprising n-butanol or a butanol isomer mixture (for example a mixture of n-butanol and 2-butanol and/or isobutanol), N-methylmorpholine and acetic acid, preferably under reflux conditions.

In a further embodiment of the invention, the conversion of XVIII is carried out by treatment with an activating agent in the presence of a base. In this case, R^{x} is hydrogen. An example of a suitable activating agent is di-(N-succinimidinyl) carbonate. Suitable activating agents are furthermore polystyrene- or not-polystyrene-bound dicyclohexylcarbodiimide (DCC), diisopropylcarbodiimide, 1-ethyl-3-(dimethylaminopropyl)carbodiimide (EDAC), carbonyldiimidazole (CDI), chloroformic esters, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride. A further suitable activating agent is O-(7-azabenzotriazole-1-yl)-N,N,N′,N′-tetramethyluronium hexafluorophosphate (HATU). Suitable bases are the compounds cited for the basic cyclization. In one embodiment, the base used is triethylamine or N-ethyldiisopropylamine or mixtures thereof, particularly preferably N-ethyldiisopropylamine.

In a further embodiment of the invention, the conversion of XVIII is carried out exclusively by heating the reaction mixture (thermal cyclization). Here, the reaction is usually carried out at temperatures in the range of from 10° C. to the boiling point of the reaction mixture, preferably from 50° C. to the boiling point of the reaction mixture, particularly preferably at the boiling point of the reaction mixture under reflux. The reaction is generally carried out in a solvent, preferably in an inert organic solvent.

In principle, suitable solvents are those solvents which can be used for the basic cyclization. Preference is given to polar aprotic solvents, for example dimethyl sulfoxide or dimethylformamide or mixtures thereof. In a preferred embodiment, the reaction is carried out in dimethyl sulfoxide.

For their part, the compounds of the formula XVIII can be prepared by the scheme shown below analogously to processes from the literature, for example according to Wilford L. Mendelson et al., Int. J. Peptide & Protein Research 35(3), (1990), 249-57, Glenn L. Stahl et al., J. Org. Chem. 43(11), (1978), 2285-6 or A. K. Ghosh et al., Org. Lett. 3(4), (2001), 635-638.

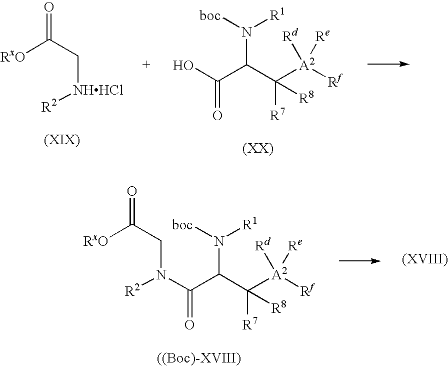

In the scheme shown above, the variables A², R^{x}, R¹, R², R⁷, R⁸ and R^{d} to R^{f} have one of the meanings given above. The synthesis comprises, in a first step, the coupling of glycine ester compounds of the formula XIX with Boc-protected compounds of the formula XX in the presence of an activating agent. Instead of Boc, it is also possible to use another amino-protective group.

With respect to suitable conditions for reacting a compound of the formula XIX with a compound of the formula XX, reference is made to the reaction of compounds VII with compounds VIII to give compounds of the formula VI.

If the groups R¹ and R² in the compounds XVII are hydrogen, the compounds XVII can also be prepared by intermolecular cyclization of a glycine ester derivative XIXa with a compound XXa according to the scheme below:

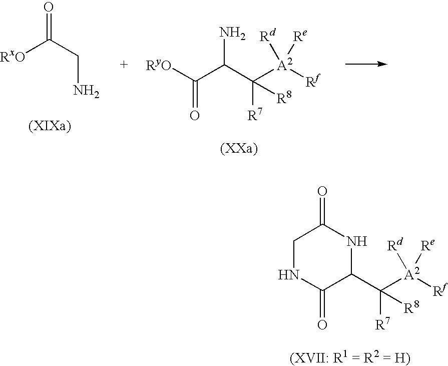

In the schemes, R^{x}, R⁷, R⁸ and R^{d} to R^{f have} one of the meanings given above. R^{y} is alkyl, for example methyl or ethyl. The intermolecular cyclization can be effected, for example, by a base, for example ammonia. The compounds XIXa and/or XXa can also be employed in the form of their acid addition salts, for example as hydrochlorides.

Process G

According to a further embodiment (hereinbelow referred to as process G), the preparation of the compounds I in which Y¹ and Y² are O and R³ together with R⁵ is a chemical bond comprises
i) providing a compound of the general formula XXI

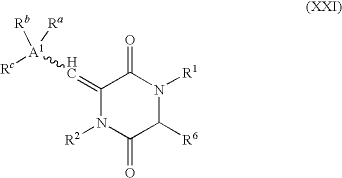

in which A¹, R², R⁶ and R^{a} to R^{c} have one of the meanings mentioned above and R¹ has one of the meanings mentioned above which is different from hydrogen or is a protective group;
ii) reacting the compound XXI in the presence of a base with the benzyl compound of the formula XXII

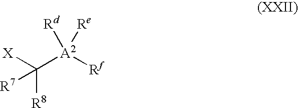

in which A², R⁷, R⁸ and R^{d} to R^{f} have one of the meanings given above and X is a nucleophilically displaceable leaving group; and
iii) if R¹ is a protective group, removing the protective group.

In formula XXI, R¹ has preferably one of the meanings given for R¹ which is different from hydrogen. In formula XXII, the variable X has preferably one of the following meanings: halogen, in particular chlorine, bromine or iodine, or O—SO₂—R^{m} where R^{m} has the meaning C₁-C₄-alkyl or aryl, which are optionally substituted by halogen, C₁-C₄-alkyl or halo-C₁-C₄-alkyl. Suitable protective groups for the nitrogen atoms of the piperazine ring in XXI are in particular the radicals C(O)R²¹ mentioned above, for example the acetyl radical.

The reaction of the compound XXI with the compound XXII in step ii) can be carried out, for example, analogously to the method described in J. Am. Chem. Soc. 105, 1983, 3214. In a preferred embodiment, the reaction is carried out in the presence of sodium hydride as base in N-methylpyrrolidone as solvent.

The compounds XXI can be provided, for example, by reacting the compound XXIII with an aldehyde compound XXIV, as illustrated in the scheme below.

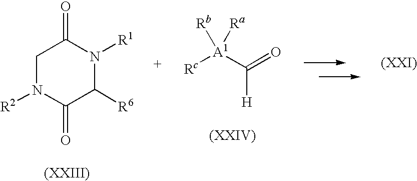

Here, A¹, R¹, R⁶ and R^{a} to R^{c} have one of the meanings mentioned above. R² has one of the meanings given above or is a protective group. Suitable protective groups for the nitrogen atoms of the piperazine ring in XXIII are in particular the radicals C(O)R²¹ mentioned above, for example the acetyl radical. R¹ and R² are in particular one of the radicals C(O)R²¹ mentioned above, for example acetyl radicals.

The reaction of XXIII with XXIV can be carried out under the conditions of an aldol condensation, as already described above. Such aldol condensations can be carried out analogously to the process described in J. Org. Chem. 2000, 65 (24), 8402-8405, Synlett 2006, 677 and J. Heterocycl. Chem. 1988, 25, 591, the entire contents of which are incorparated herein by way of reference.

The reaction is generally carried out in the presence of a base. The base used is preferably an alkali metal or alkaline earth metal carbonate, for example sodium carbonate, potassium carbonate or cesium carbonate, or mixtures thereof.

The reaction is preferably carried out in an inert, preferably aprotic organic solvent. Examples of suitable solvents are in particular dichloromethane, dichloroethane, chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide, N-methylpyrrolidone and dimethylacetamide.

The compounds reacted are preferably those compounds XXIII in which R¹ and R² are a protective group and in particular an acyl radical R²¹C(O)—(R²¹═C₁-C₄-alkyl), for example an acetyl radical. Accordingly, the condensation reaction is generally followed by a removal of the protective groups. The removal of a protective group R¹, R² can be carried out analogously to known processes of protective group chemistry, for example by the method described in Green, Wuts, Protective Groups in Organic Synthesis, 3rd ed. 1999, John Wiley and Sons, p. 553. A subsequent alkylation for introducing the radicals R¹ and/or R² can be carried out be the method given above.

The compounds XXIII are known. Their preparation can be carried out analogously to the preparation of the compounds XVII described above, according to the scheme shown below:

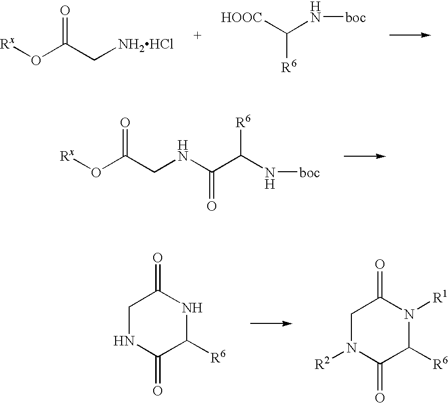

In this scheme, R¹, R² and R⁶ have one of the meanings mentioned above. R^{x} is preferably C₁-C₄-alkyl or benzyl. Boc is a tert-butoxycarbonyl radical.

With respect to further details for the first reaction step, reference is made to the reaction of compound XIX or XIXa with the compound XX or XXa. The subsequent removal of the Boc protective group can be carried out analogously to the conversion of the compound IX into the compound VII. The cyclization of the resulting deprotected compound can be carried out using the methods mentioned for the cyclization of the compound XVIII. If R¹ and R² are a protective group, for example a radical C(O)R²¹, these protective groups can be introduced analogously to known processes of protective group chemistry, for example by reaction with anhydrides of the formula (R²¹C(O))₂O, for example by the method described in Green, Wuts, Protective Groups in Organic Synthesis, 3rd ed. 1999, John Wiley and Sons, p. 553.

Process H

Compounds of the formula I in which R³ and R⁵ are hydrogen can be prepared by hydrogenation of compounds of the formula I in which R³ together with R⁵ is a chemical bond.

The hydrogenation can be carried out analogously to known processes for reducing C═C double bonds (see, for example, J. March, Advanced Organic Chemistry, 3rd ed. John Wiley & Sons 1985, pp. 690-700, and also Peptide Chemistry 17, 1980, pp. 59-64, Tetrahedron Lett. 46, 1979, pp. 4483-4486).

Frequently, the hydrogenation is carried out by reaction with hydrogen in the presence of transition metal catalysts, for example catalysts comprising Pt, Pd, Rh or Ru as active metal species. Suitable are both heterogeneous catalysts, such as supported Pd or Pt catalysts, for example Pd on carbon, furthermore PtO₂, and also homogeneous calysts. The use of stereoselective catalysts permits an enantioselective hydrogenation of the double bond (see Peptide Chemistry 17, 1980, pp. 59-64, Tetrahedron Lett. 46, 1979, pp. 4483-4486).

The hydrogenation can be carried out in an analogous manner with compounds of the formula XVI, i.e. before a radical R⁶ different from hydrogen is introduced.

If the hydrogenation affords compounds of the formula I or XVI in which R¹ and/or R² are/is hydrogen, these compounds can be converted under the conditions shown above into the compounds of the formula I or XVI in which R¹ and R² have one of the meanings given above.

Process I

Compounds of the formula I in which Y¹ and/or Y² are/is sulfur can be obtained, for example, by reacting the corresponding compounds of the formula I in which Y¹ and/or Y² are/is oxygen with a sulfurizing agent.

Examples of suitable sulfurizing agents are organophosphorus sulfides, such as Lawesson's reagent (2,2-bis-(4-methoxyphenyl)-1,3,2,4-dithiadiphosphetane-2,4-disulfid), organotin sulfides, such as bis(tricyclohexyltin) sulfide or phosphorus pentasulfide (see also J. March, Advanced Organic Synthesis, 4. edition, Wiley Interscience 1992, p. 893 f and the literature cited therein). The reaction can be carried out in a solvent or in the absence of a solvent. Suitable solvents are inerte organic solvents known from the prior art and in particular pyridine and comparable solvents. The temperature required for the reaction is generally above room temperature and in particular in the range of from 50 to 200° C.

Process J

Compounds of the formula I in which Y¹ and/or Y² are/is a group NR^{y1} or NR^{y2} can be prepared, for example, by reacting the corresponding compounds of the formula I in which Y¹ and/or Y² are/is oxygen under dehydrating conditions with primary amines of the formula H₂NR^{y1} or H₂NR^{y2}.

Process K

Compounds of the formula I in which R¹ together with R² is a 1-, 2-, 3- or 4-membered carbon chain in which one carbon atom may be replaced by O, S or a group NR^{A} can be prepared, for example, from precursors of the compounds I in which R¹ and R² are hydrogen by reaction with a compound of the formula X^{a}-A-X^{a} in which A is the 1-, 2-, 3- or 4-membered carbon chain in which one carbon atom may be replaced by O, S or a group NR^{A} and X^{a} is a suitable leaving group, such as, for example, iodine.

Process L

Compounds of the formula I in which R³ together with R⁵ is a 1-, 2-, 3- or 4-membered carbon chain in which one carbon atom may be replaced by O, S or a group NR^{I} can be prepared, for example, from compounds I in which R³ together with R⁵ is a chemical bond.

For example, a ring can be constructed by addition to the double bond using a suitable elektrophile. Thus, compounds I in which R³ together with R⁵ is an oxygen atom can be obtained by epoxidation of the corresponding unsaturated compound.

Compounds I in which R³ together with R⁵ is an optionally substituted methylene group can be obtained by known cyclopropanation reactions, for example by addition of carbenes or carbenoids to the exocyclic double bond in the compounds I-A.

In an analogous manner, it is also possible to prepare compounds I in which R⁶ and R⁸ are a 1-, 2-, 3- or 4-membered carbon chain in which one carbon atom may be replaced by O, S or a group NR^{I}.

Process M

Compounds of the formula I in which R^{a} together with R⁴ is a 2-, 3-, 4- or 5-membered carbon chain in which one carbon atom may be replaced by O, S or a group NR^{L} where one of the carbon atoms may carry a carbonyl oxygen atom can be prepared, for example, by intramolecular Michael addition from compounds of the formula I in which R^{a} is a carboxyl group and R³ together with R⁵ is a chemical bond. The conditions customary for this are known to the person skilled in the art.

Process N

Compounds of the formula I in which R^{a} together with R² is a chemical bond or a 1-, 2-, 3- or 4-membered carbon chain can be prepared, for example, by reaction under basic conditions from precursors of the compounds I in which R² is hydrogen and R^{a} is halogen, in particular fluorine. Suitable reaction conditions are those mentioned above for the reaction of the secondary amino groups with appropriate alkylating agents.

Process O

Compounds of the formula I in which R² together with R⁵ and together with the atoms to which these radicals are attached are a cyclische group can be prepared, for example, from cyclic amino acids, such as proline.

In an analogous manner, it is also possible to prepare compounds of the formula I in which R¹ together with R⁶ and together with the atoms to which these radicals are attached are a cyclic group from cyclic amino acids.

Process P

Compounds of the formula I in which R³ together with R⁴ and/or R⁷ together with R⁸ is a cyclic group can be prepared from corresponding phenyl-substituted compounds of the formula V or XXII.

Process Q

The preparation of the compound I-A in which Y¹ and Y² are oxygen can furthermore be carried out analogously to the illustrated preparation of compound XVI by aldol addition and subsequent elimination of water or preferably be reaction under the conditions of an aldol condensation according to the synthesis illustrated in process F.

The compounds I and their agriculturally useful salts are suitable, both in the form of isomer mixtures and in the form of the pure isomers, as herbicides. They are suitable as such or as an appropriately formulated composition. The herbicidal compositions comprising the compound I or la control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds of the formula I, or compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapsis alba, Solanum tuberosum, Sorghum bicolor (s. vulgare), Theobroma cacao, Trifolium pratense, Triticum aestivum, Triticale, Triticum durum, Vicia faba, Vitis vinifera and Zea mays.

In addition, the compounds of the formula I may also be used in crops which tolerate the action of herbicides owing to breeding, including genetic engineering methods.

In addition, the compounds of the formula I can also be used in crops which tolerate insects or fungal attack as the result of breeding, including genetic engineering methods.

Furthermore, it has been found that the compounds of the formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of the formula I.

As desiccants, the compounds of the formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pernicious fruit, stone fruit and nuts. The same mechanism, i.e. the promotion of the development of abscission tissue between fruit part or leaf part and shoot part of the plants is also essential for the controlled defoliation of useful plants, in particular cotton.

Moreover, a shortening of the time interval in which the individual cotton plants mature leads to an increased fiber quality after harvesting.

The compounds of the formula I, or the herbicidal compositions comprising the compounds of the formula I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading or watering or treatment of the seed or mixing with the seed. The use forms depend on the intended purpose; in any case, they should ensure the finest possible distribution of the active ingredients according to the invention.

The herbicidal compositions comprise a herbicidally effective amount of at least one compound of the formula I or an agriculturally useful salt of compounds of the formula II, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, optionally colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Kelzan® from Kelco), Rhodopol® 23 (Rhone Poulenc) or Veegum® (from R. T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia); long-chain alcohols, fatty acids, salts of fatty acids, organofluorine compounds and mixtures thereof.

Bactericides can be added for stabilizing the aqueous herbicidal formulations. Examples of bactericides are bactericides based on diclorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF AG), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octyiphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denaturated proteins, polysaccharides (e.g. methylcellulose), hydrophobically modified starches, polyvinyl alcohol (Mowiol types Clariant), polycarboxylates (BASF AG, Sokalan types), polyalkoxylates, polyvinylamine (BASF AG, Lupamine types), polyethyleneimine (BASF AG, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of the formula I or Ia, either as such or dissolved in an oil or solvent, can be homogenized in water by means of a wetting agent, tackifier, dispersant or emulsifier. Alternatively, it is also possible to prepare concentrates comprising active compound, wetting agent, tackifier, dispersant or emulsifter and, if desired, solvent or oil, which are suitable for dilution with water.

The concentrations of the compounds of the formula I in the ready-to-use preparations can be varied within wide ranges. In general, the formulations comprise approximately from 0.001 to 98% by weight, preferably 0.01 to 95% by weight of at least one active ingredient. The active ingredients are employed in a purity of from 90% to 100%, preferably 95% to 100% (according to NMR spectrum).

The compounds I according to the invention can for example be formulated as follows:

1. Products for Dilution with Water

A Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

B Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

C Emulsifiable Concentrates

15 parts by weight of active compound are dissolved in 75 parts by weight of an organic solvent (eg. alkylaromatics) with addition of calcium dodecylbenzenesulfonate and castor oil ethoxylate (in each case 5 parts by weight). Dilution with water gives an emulsion. The formulation has an active compound content of 15% by weight.

D Emulsions

25 parts by weight of active compound are dissolved in 35 parts by weight of an organic solvent (eg. alkylaromatics) with addition of calcium dodecylbenzenesulfonate and castor oil ethoxylate (in each case 5 parts by weight). This mixture is introduced into 30 parts by weight of water by means of an emulsifier (Ultraturrax) and made into a homogeneous emulsion. Dilution with water gives an emulsion. The formulation has an active compound content of 25% by weight.

E Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

H Gel Formulations

In a ball mill, 20 parts by weight of active compound, 10 parts by weight of dispersant, 1 part by weight of gelling agent and 70 parts by weight of water or of an organic solvent are mixed to give a fine suspension. Dilution with water gives a stable suspension with active compound content of 20% by weight.

2. Products to be Applied Undiluted

I Dusts

5 parts by weight of active compound are ground finely and mixed intimately with 95 parts by weight of finely divided kaolin. This gives a tracking powder with an active compound content of 5% by weight.

J Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

K ULV Solutions (UL)

10 parts by weight of active compound are dissolved in 90 parts by weight of an organic solvent, for example xylene. This gives a product to be applied undiluted with an active compound content of 10% by weight.

The compounds of the formula I or the herbicidal compositions comprising them can be applied pre- or post-emergence, or together with the seed of a crop plant. It is also possible to apply the herbicidal composition or active compounds by applying seed, pretreated with the herbicidal compositions or active compounds, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the compounds of the formula I or the herbicidal compositions can be applied by treating seed.

The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the compounds of the formula I according to the invention or the compositions prepared therefrom. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The seed used can be seed of the useful plants mentioned above, but also the seed of transgenic plants or plants obtained by customary breeding methods.

The rates of application of the active compound are from 0.001 to 3.0, preferably 0.01 to 1.0, kg/ha of active substance (a.s.), depending on the control target, the season, the target plants and the growth stage. To treat the seed, the compounds I are generally employed in amounts of from 0.001 to 10 kg per 100 kg of seed.

To widen the spectrum of action and to achieve synergistic effects, the compounds of the formula I may be mixed with a large number of representatives of other herbicidal or growth-regulating active ingredient groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, phenyipropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils, phenyl pyrazolines and isoxazolines and derivatives thereof.

It may furthermore be beneficial to apply the compounds of the formula I alone or in combination with other herbicides, or else in the form of a mixture with other crop protection agents, for example together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

It may also be advantageous to ise the compounds of the formula I in combination with safeners. Safeners are chemical compounds which prevent or reduce damage to useful plants without having any substantial effect on the herbicidal action of the compounds of the formula I on unwanted plants. They can be used both before sowing (for example for the treatment of seed, for cuttings or for seedlings) and for pre- or post-emergence treatment of the useful plant. The safeners and the compounds of the formula I can be applied simultaneously or in succession. Suitable safeners are, for example, (quinolin-8-oxy)acetic acids, 1-phenyl-5-haloalkyl-1H-1,2,4-triazole-3-carboxylic acids, 1-phenyl-4,5-dihydro-5-alkyl-1H-pyrazol-3,5-dicarboxylic acids, 4,5-dihydro-5,5-diaryl-3-isoxazolcarboxylic acids, dichloroacetamides, alpha-oximinophenylacetonitrile, acetophenonoximes, 4,6-dihalo-2-phenylpyrimidines, N-[[4-(aminocarbonyl)phenyl]-sulfonyl]-2-benzamides, 1,8-naphthoic anhydride, 2-halo-4-(haloalkyl)-5-thiazolcarboxylic acids, phosphorothiolates and O-phenyl N-alkylcarbamates and also their agriculturallt useful salts and, provided they have an acid function, their agriculturally useful derivatives, such as amides, esters and thioesters.

Hereinbelow, the preparation of piperazine compounds of the formula I is illustrated by examples; however, the subject matter of the present invention is not limited to the examples given.

### EXAMPLES

The products shown below were characterized by determination of the melting point, by NMR spectroscopy or by the masses determined by HPLC-MS spectrometry ([m/z]) or by the retention time (RT; [min.]).

[HPLC-MS=high performance liquid chromatography coupled with mass spectrometry; unless stated the contrary: HPLC column: RP-18 column (Chromolith Speed ROD from Merck KgaA, Germany), 50×4.6 mm; mobile phase: acetonitrile+0.1% trifluoroacetic acid (TFA)/water+0.1% TFA, gradient from 5:95 to 100:0 over 5 minutes at 40° C., flow rate 1.8 ml/min;

MS: quadrupole electrospray ionisation, 80 V (positive mode).]

### I. PREPARATION EXAMPLES

### EXAMPLE 1a/1b

### 2-(5-Benzyl-1,4-dimethyl-5-methylsulfanyl-3,6-dioxopiperazin-2-ylidenemethyl)benzonitrile

### 1.1 Preparation of methyl(2-tert-butoxycarbonylamino-3-phenylpropionylamino)-acetate

At 0° C., ethyldiisopropylamine (259 g, 2.0 mol), N-tert-butoxycarbonyl-L-phenylalanine (212 g, 0.8 mol) and 1-ethyl-3-(3′-dimethylamino-propyl)carbodiimide (EDAC, 230 g, 1.2 mol) were added to a solution of methyl glycinate hydrochloride (100 g, 0.8 mol) in tetrahydrofuran (THF, 1000 ml). The reaction mixture was then stirred at room temperature for 24 h. The resultion reaction mixture was freed from volatile components under reduced pressure, and the residue obtained in this manner was taken up in water (1000 ml). The aqueous phase was extracted repeatedly with CH₂Cl₂. The organic phases obtained in this manner were combined, washed with water, dried over Na₂SO₄ and filtered, and the solvent was removed under reduced pressure. Methyl (2-tert-butoxycarbonylamino-3-phenylpropionylamino)acetate was obtained as a yellow oil in an amount of 300 g. The resulting crude product was reacted further without further purification.

### 1.2 Preparation of 3-benzylpiperazine-2,5-dione

At room temperature, trifluoroacetic acid (342 g, 3 mol) was added dropwise to a solution of methyl(2-tert-butoxycarbonylamino-3-phenylpropionylamino)acetate (300 g, about 0.8 mol) in CH₂Cl₂. The resulting reaction mixture was stirred at room temperature for 24 h and then concentrated under reduced pressure. The residue obtained was taken up in THF (500 ml), and an aqueous ammonia solution (25% strength, 500 ml) was added slowly. The reaction mixture was stirred at room temperature for a further 72 h. The precipitated solid was isolated by filtration and washed with water. 3-Benzylpiperazine-2,5-dione was obtained in an amount of 88 g (yield 54%).

### 1.3 Preparation of 1,4-diacetyl-3-benzylpiperazine-2,5-dione

A solution of 3-benzylpiperazine-2,5-dione (20.4 g, 0.1 mol) in acetic anhydride (200 ml) was stirred under reflux conditions for 4 h. The reaction mixture obtained was concentrated under reduced pressure. The residue was taken up in CH₂Cl₂, washed successively with an aqueous NaHCO₃ solution and water, dried over Na₂SO₄ and filtered, and the solvent was removed under reduced pressure. 1,4-Diacetyl-3-benzylpiperazine-2,5-dione was obtained as a yellow oil in an amount of 28.5 g (quantitative) and reacted further as crude product.

HPLC-MS [m/z]: 289.1 [M+1]⁺.

### 1.4 Preparation of 1-acetyl-6-benzyl-3-(2-bromobenzylidene)piperazine-2,5-dione

2-Bromobenzaldehyde (5.55 g, 0.03 mol) and Cs₂CO₃ (9.8 g, 0.03 mol) were added to a solution of 1,4-diacetyl-3-benzylpiperazine-2,5-dione (17.4 g, 0.06 mol) in dimethylformamide (DMF, 100 ml). The reaction mixture was stirred at room temperature for 36 h, water (500 ml) and citric acid (10 g) were then added and the mixture was extracted repeatedly with CH₂Cl₂. The organic phases obtained in this manner were combined, washed with water, dried over Na₂SO₄ and filtered, and the solvent was removed under reduced pressure. Following purification by column chromatography (mobile phase: CH₂Cl₂), 1-acetyl-6-benzyl-3-(2-bromobenzylidene)piperazine-2,5-dione was obtained as a yellow oil in an amount of 12 g (yield 48%).

HPLC-MS [m/z]: 413.9 [M+1]⁺.

### 1.5 Preparation of 3-benzyl-6-(2-bromobenzylidene)piperazine-2,5-dione

Dilute aqueous hydrochloric acid (5% strength, 250 ml) was added to a solution of 1-acetyl-6-benzyl-3-(2-bromobenzylidene)piperazine-2,5-dione (12 g, 0.03 mol) in THF (50 ml). The reaction mixture was stirred under reflux conditions for 8 h. After cooling of the reaction solution, the precipitated solid was isolated by filtration. The solid obtained in this manner was washed with water and THF. 3-Benzyl-6-(2-bromobenzylidene)piperazine-2,5-dione was obtained as a colorless solid in an amount of 8.3 g (yield 75%).

HPLC-MS [m/z]: 371.2 [M]⁺.

### 1.6 3-Benzyl-6-(2-bromobenzylidene)-1,4-dimethylpiperazine-2,5-dione

At 0° C., NaH (0.8 g, 60% pure, 0.02 mol) was added to 3-benzyl-6-(2-bromobenzylidene)piperazine-2,5-dione (3.71 g, 0.01 mol) in N,N-dimethylformamide (DMF) (50 ml). The mixture was stirred at 0° C. for 1 h, and methyl iodide (14.2 g, 0.1 mol) was then added. The reaction mixture was stirred at room temperature for 18 h and then introduced into a solution of water (500 ml) and citric acid (5 g). The mixture was extracted repeatedly with CH₂Cl₂. The organic phase obtained in this manner was washed with water, dried over Na₂SO₄, filtered and concentrated. Trituration with diisopropyl ether gave the title compound (2 g, 50% yield).

HPLC-MS [m/z]: 401.4 [M+1]⁺.

### 1.7 2-(5-Benzyl-1,4-dimethyl-3,6-dioxopiperazin-2-ylidenemethyl)benzonitrile

CuCN (0.9 g, 0.01 mol) was added to 3-benzyl-6-(2-bromobenzylidene)-1,4-dimethylpiperazine-2,5-dione (2 g, 0.005 mol) in N-methylpyrrolid-2-one (NMP) (20 ml). The mixture was stirred at 150° C. for 18 h. The mixture was then introduced into a solution of water (50 ml) and NaCN (3 g). The mixture was extracted repeatedly with CH₂Cl₂. The organic phase obtained in this manner was washed with water, dried over Na₂SO₄, filtered and concentrated. Following purification by column chromatography and trituration with diisopropyl ether, the desired product was obtained as a beige solid (1.2 g, 67%).

HPLC-MS [m/z]: 346.4 [M+1]⁺.

### 1.8 2-(5-Benzyl-1,4-dimethyl-5-methylsulfanyl-3,6-dioxopiperazin-2-ylidenemethyl)-benzonitrile

At −78° C., lithium hexamethyldisilazide (LHMDS) (1.06 M in THF, 5.3 ml, about 5.6 mmol) was added dropwise under argon to 2-(5-benzyl-1,4-dimethyl-3,6-dioxopiperazin-2-ylidenemethyl)benzonitrile (Z isomer) (1.5 g, 4.3 mmol) in abs. THF (25 ml). The mixture was stirred at −78° C. for 1 h, and methyl methanethiol-sulfonate (1.7 g, 13.5 mmol, in 1 ml THF) was then added. The reaction mixture was stirred at 0° C. for 1 h and at room temperature for 12 h and then quenched with citric acid solution (5%). CH₂Cl₂ was added, and the mixture was washed repeatedly with H₂O. The organic phase obtained in this manner was dried over Na₂SO₄, filtered and concentrated. After flash chromatography (hexanr:Methyl tert-butyl ether 1:1→100% methyl tert-butyl ether), the title compound was obtained an an E/Z isomer mixture in the form of a colorless solid (158 mg, 9%) of melting point 161° C. (E/Z about 1:3).

### EXAMPLE 2

### 2-(5-Benzyl-1,4-dimethyl-5-allyl-3,6-dioxo-piperazin-(Z)-2-ylidenemethyl)-benzonitrile

At 0° C., sodium hydride (45 mg, 60% pure, about 1,1 mmol) was added to 2-(5-benzyl-1,4-dimethyl-3,6-dioxopiperazin-2-ylidenemethyl)benzonitrile (0.3 g, 0.87 mmol) from Example 1.7 in DMF (10 ml). The mixture was stirred at 0° C. for 1 h, and allyl bromide (250 mg, 2.1 mmol) was then added. The reaction mixture was stirred at 0° C. for 1 h and at room temperature for 1 h and then quenched with water (50 ml). The aqueous reaction mixture was extracted repeatedly with tert-butyl methyl ether. The organic phase obtained was washed with water, dried over sodium sulfate, filtered and concentrated. After purification by column chromatography, this gave the title compound as a colorless solid of melting point 123° C. (173 mg, 52%).

HPLC-MS [m/z]: 386.4 [M+1]⁺.

### EXAMPLE 3

### 2-(5-Benzyl-1,4-dimethyl-5-methylsulfonyl-3,6-dioxo-piperazin-(Z)-2-ylidenemethyl)benzonitrile

Sodium tungstate dihydrate (10 mg, 0.03 mmol) and glacial acetic acid (3 ml) were added to 2-(5-benzyl-1,4-dimethyl-5-methylsulfanyl-3,6-dioxopiperazin-2-ylidenemethyl)benzonitrile from Example 1 (Z isomer, 90 mg, 0.23 mmol). Hydrogen peroxide (60 mg, 30% strength solution, 0.53 mmol) was then added dropwise at room temperature, and the mixture was stirred overnight. Water was then added, and the reaction mixture was neutralized with sodium bicarbonate. The mixture was extracted three times with ethyl acetate, and the organic phase was washed with sat. sodium thiosulfate solution until free of peroxide and extracted three times with water. After drying of the organic phase and concentration, the residue obtained was subjected to flash chromatography (hexane:methyl tert-butyl ether 1:1→100% methyl tert-butyl ether), which gave the title compound (20 mg, 21%) as a yellow solid.

### EXAMPLE 4

### 2-[5-Trifluoroacetyl-5-benzyl-1,4-dimethyl-3,6-dioxopiperazin-(Z)-2-ylidenemethyl]benzonitrile

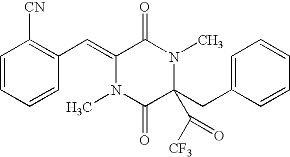

At 0° C. and under an atmosphere of argon, LHMDS (1.06 M in THF, 1.5 ml, 1.6 mmol) was slowly added dropwise to 2-(5-benzyl-1,4-dimethyl-3,6-dioxopiperazin-2-ylidenemethyl)benzonitrile from Example 1.7 (Z isomer, 300 mg, 0.9 mmol) in 10 ml of abs. THF. The mixture was then stirred for 15 min, and trifluoroacetic anhydride (1 g, 4.8 mmol) was then added dropwise. The mixture was stirred at 0° C. for 1 h and at room temperature overnight. Methyl tert-butyl ether was added, and the reaction mixture was extracted three times with water, dried and concentrated. The residue obtained was subjected to flash chromatography (hexane:methyl tert-butyl ether 1:1→100% methyl tert-butyl ether), which gave the title compound (80 mg) as a yellow foam of melting point 54° C.

### EXAMPLE 5

### 2-(5,5-Dibenzyl-1,4-dimethyl-3,6-dioxo-piperazin-(Z)-2-ylidenmethyl)-benzonitril

At 0° C., sodium hydride (45 mg, 60% pure, about 1.1 mmol) was added to 2-(5-benzyl-1,4-dimethyl-3,6-dioxopiperazin-2-ylidenemethyl)benzonitrile (Z isomer, 0.3 g, 0.9 mmol) from Example 1.7 in DMF (10 ml). The mixture was stirred at 0° C. for 1 h, and benzyl bromide (350 mg, 2 mmol) was then added. The reaction mixture was stirred at 0° C. for 1 h and at room temperature for 48 and then quenched with water (50 ml). The aqueous reaction mixture was extracted three times with tert-butyl methyl ether. The resulting organic phase was washed three times with water, dried over sodium sulfate, filtered and concentrated. After purification by flash chromatography (hexane:ethyl acetate 3:1), this gave the title compound as a yellow solid of melting point 175° C. (182 mg, 46%).

The preparation of the compounds of the formula I-A.a′ (examples 6, 7, 8, 9, 10, and 11a/11b) compiled in Table B was carried out analogously to Examples 1a/1b, 2, 3, 4 or 5 shown above.

TABLE B
Compounds of the general formula I-A.a′ in which R^{c} and R^{d} are each
hydrogen and R¹ is methyl.

| | |
|---|---|
| | I-A.a′ |
| 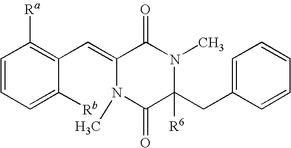 | |

| | | | | RT HPLC/MS and/or | |
|---|---|---|---|---|---|
| Example | R^{a} | R^{b} | R⁶ | m.p. | Isomer* |
| 1a | CN | H | SCH₃ | 3.311 min; m/z = 414.0 | isomer 1 |
| | | | | [M + Na]⁺ | |
| 1b | CN | H | SCH₃ | 3.275 min; m/z = 414.0 | isomer 2 |
| | | | | [M + Na]⁺ | |
| 2 | CN | H | CH—CH═CH₂ | 3.227 min; m/z = 386.4 | Z isomer |
| | | | | [M + H]⁺ | |
| | | | | 123° C. | |
| 3 | CN | H | —SO₂CH₃ | 3.286 min; m/z = 446.0 | Z isomer |
| | | | | [M + Na]⁺ | |
| 4 | CN | H | —C(O)CF₃ | 3.280 min; m/z = 441.9 | Z isomer |
| | | | | [M + H]⁺/54° C. | |
| 5 | CN | H | benzyl | 175° C. | Z isomer |
| 6 | CN | H | 2-propynyl | 3.252 min; m/z = 385 | Z-isomer |
| | | | | [M + H]⁺ | |
| 7 | NO₂ | H | 2-propynyl | 3.366 min; m/z = 403.9 | Z isomer |
| | | | | [M + H]⁺ | |
| 8 | Br | OCH₃ | COOCH₃ | 2.673 min; m/z = 414 | Z isomer |
| | | | | [M + H]⁺ | |
| 9 | CN | H | CH₂NHCOCH₃ | 2.716 min; m/z = 417.1 | Z isomer |
| | | | | [M + H]⁺ | |
| 10 | CN | H | OCH₃ | 2.692 min; m/z = 375.8 | Z isomer |
| | | | | [M]+ | |
| 11a | CN | H | COOCH₃ | 5.390 min **); | Z isomer |
| | | | | m/z = 404.07 [M + H]+ | |
| 11b | CN | H | COOCH₃ | 6.075 min **); | E isomer |
| | | | | m/z = 404.07 [M + H]+ | |

*) This statement refers to the stereochemistry of the double bond at the piperazine skeleton; isomer 1 or isomer 2 is an essentially pure isomer to which no configuration has been assigned.
**) HPLC-column: RP-18 column (XTerra MS 5 mm from Waters) Eluent: acetonitrile + 0.1% formic acid (A)/water + 0.1% formic acid in (B). Gradient: from 5:95 (A/B) to 100:0 (A/B) in 8 minutes, at room temperature.
MS: Quadrupol Electrospray Ionisation, 80 V (positive mode)
m.p. melting point

### EXAMPLE 12

### 2-[5-Amino-5-benzyl-1,4-dimethyl-3,6-dioxopiperazin-(2Z)-ylidenemethyl]-benzonitrile

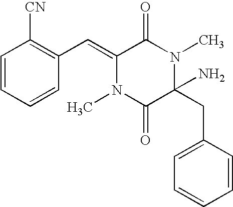

At −30° C., a 0.5 M solution of potassium hexamethyldisilazide (KHMDS) in toluene (11.6 ml, 5.8 mmol) was added dropwise to a mixture of 2-[5-benzyl-1,4-dimethyl-3,6-dioxopiperazin-(2Z)-ylidenemethypenzonitrile from example 1.7 (2 g, 5.8 mmol) in dry THF (23 ml) and 1,3-dimethyl-3,4,5,6-tetrahydro-2(1H)-pyrimidinone (DMPU) (34 ml). The reaction mixture was stirred at −30° C. for 3 hours. A solution of 2,4,6-triisopropylbenzenesulfonyl azide (2.3 g, 7.4 mmol) in THF (10 ml) was then added. The reaction mixture was stirred at −30° C. for further 3 hours and then allowed to warm up to room temperature. A phosphate buffer solution (pH=7, 50 ml) was added and then the mixture was extracted repeatedly with CH₂Cl₂. The organic phase obtained was dried over Na₂SO₄, filtered and freed from solvent under reduced pressure. The crude azide obtained was used in the next step without further purification.

A mixture of the azide in DMPU and 10% Pd/C (22 mg) was diluted with ethanol (20 ml) and stirred under hydrogen atmosphere at room temperature for 12 h. The reaction mixture was filtered through Celite, the filtrate freed from solvent under reduced pressure, the solid obtained stirred with methanol and then filtered. After column chromatography (silical gel, hexane/ethyl acetate), 2-[5-amino-5-benzyl-1,4-dimethyl-3,6-dioxopiperazin-(2Z)-ylidenemethypenzonitrile was obtained in an amount of 710 mg (35% yield, based on two steps).

RT HPLC-MS: 4.050 [m/z]: 344.1 [M−NH₃+H]⁺

HPLC-column: RP-18 column (XTerra MS 5 mm from Waters) Eluent: acetonitrile+0.1% formic acid (A)/water+0.1% formic acid in (B). Gradient: from 5:95 (A/B) to 100:0 (A/B) in 8 minutes, at room temperature.

MS: Quadrupol Electrospray Ionisation, 80 V (positive mode)

### EXAMPLE 13

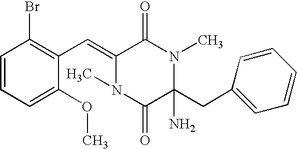

The preparation of the title compound was carried out analogously to Example 12.

HPLC-MS RT: 5.551 min; m/z=427.1 [M−NH₃+H]⁺

HPLC-column: RP-18 column (XTerra MS 5 mm from Waters) Eluent: acetonitrile+0.1% formic acid (A)/water+0.1% formic acid in (B). Gradient: from 5:95 (A/B) to 100:0 (A/B) in 8 minutes, at room temperature.

MS: Quadrupol Electrospray Ionisation, 80 V (positive mode)

### EXAMPLE 14

### 3-(Bromo-phenyl-methyl)-3-hydroxy-1,4-dimethyl-6-[1-(2-nitrophenyl)-meth-(Z)-ylidene]piperazine-2,5-dione

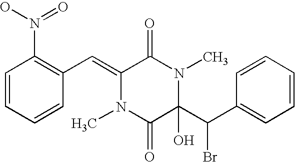

### 14.1 1,4-Diacetyl-3-[1-phenyl-meth-(Z)-ylidene]piperazine-2,5-dione

Benzaldehyde (0.03 mol) and Cs₂CO₃ (0.03 mol) were added to a solution of 1,4-diacetyl-piperazine-2,5-dione (0.06 mol) in dimethylformamide (DMF) (100 ml). The reaction mixture was stirred at room temperature for 36 h, then introduced into a solution of citric acid (10 g) in water (500 ml) and extracted repeatedly with CH₂Cl₂. The organic phase obtained were combined, washed with water, dried over Na₂SO₄, filtered and freed from solvent under reduced pressure to obtain the title compound.

### 14.2 3-[1-(2-Nitrophenyl)-meth-(Z)-ylidene]-6-[1-phenylmeth-(Z)-ylidene]-piperazine-2,5-dione

2-Nitrobenzaldehyde (3.8 g, 0.037 mol) and K₂CO₃ (5.2 g, 0.037 mol) were added to a solution of 1,4-diacetyl-3-[1-phenyl-meth-(Z)-yliden]piperazin-2,5-dione (7.17 g, 0.025 mol) in DMF (100 ml). The reaction mixture was stirred over night at room temperature, a saturated aqueous solution of citric acid was added and the mixture was extracted repeatedly with ethyl acetate. The organic phases were combined, washed with water, dried over Na₂SO₄, filtered and freed from solvent under reduced pressure. 3-[1-(2-Nitrophenyl)-meth-(Z)-ylidene]-6-[1-phenylmeth-(Z)-ylidene]-piperazine-2,5-dione were obtained as isomer mixture in an amount of 3.4 g (yield 41%) and used without further purification in the next step.

HPLC-MS [m/z]: 336.2 [M+H]⁺.

### 14.3 1,4-Dimethyl-3-[1-(2-nitrophenyl)-meth-(Z)-ylidene]-6-[1-phenyl-meth-(Z)-ylidene]-piperazine-2,5-dione

At 0° C., NaH (0.4 g, 60%, 0.01 mol) was added to a solution of 3-[1-(2-nitrophenyl)-meth-(Z)-ylidene]-6-[1-phenylmeth-(Z)-ylidene]-piperazine-2,5-dione (1.67 g, 0.005 mol) in DMF (30 ml). The reaction mixture was stirred at 0° C. for 4 h and then methyl iodide (2.13 g, 0.015 mol) was added. The reaction mixture was stirred at room temperature for further 18 hours, a saturated aqueous solution of citric acid was added and the mixture was extracted repeatedly with ethyl acetate. The organic phases were combined, washed with water, dried over Na₂SO₄, filtered and freed from solvent under reduced pressure. After column chromatography (RP, methanol/water, 7:3), 1,4-dimethyl-3-[1-(2-nitrophenyl)-meth-(Z)-ylidene]-6-[1-phenyl-meth-(Z)-ylidene]-piperazine-2,5-dione was obtained in an amount of 0.45 g (yield 24%). HPLC-MS [m/z]: 364.1 [M+H]⁺

### 14.4 3-(Bromo-phenyl-methyl)-3-hydroxy-1,4-dimethyl-6-[1-(2-nitrophenyl)-meth-(Z)-ylidene]piperazine-2,5-dione

A mixture of 1,4-dimethyl-3-[1-(2-nitrophenyl)-meth-(Z)-ylidene]-6-[1-phenyl-meth-(Z)-ylidene]-piperazine-2,5-dione (0.156 g, 0.00043 mol) and N-bromosuccinimide (NBS, 0.078 g, 0.0004 mol) in dioxane (10 ml) was stirred at room temperature for18 h. The solution was freed from solvent under reduced pressure, the residue was solved in ethyl acetate and washed several times with water. The organic phases were dried over Na₂SO₄, filtered and freed from the solvent under reduced pressure. 3-(Bromo-phenyl-methyl)-3-hydroxy-1,4-dimethyl-6-[1-(2-nitrophenyl)-meth-(Z)-ylidene]piperazine-2,5-dione was obtained as isomer mixtures (1:1) (0.191 g, 96%). The purification using column chromatography (silica gel, hexane/ethyl acetate, 2:1) afforded the compounds 14a and 14b.

Compound 14a: RT HPLC/MS: 3.055 min; m/z=462.0 [M+H]⁺

Compound 14b: RT HPLC/MS: 3.115 min; m/z=462.0 [M+H]⁺

### EXAMPLE 15

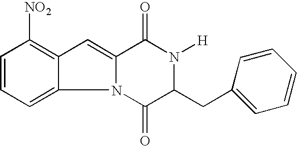

A mixture of 0.42 g 3-benzyl-6-(2-bromo-6-nitrobenzylidene)piperazine-2,5-dione and 0.18 g of CuCN in 10 ml NMP was stirred at 140° C. for 18 h. The reaction mixture was allowed to cool and introduced into 250 ml ethyl acetate. The mixture was extracted 5 times with water and the organic phase was dried over sodium sulfate, filtered and concentrated. Purification of the crude product by flash chromatography (methyl tert-butyl ether) gave a yellow solid (0.11 g).

RT HPLC/MS: 2.956 min; m/z=336.4 [M+H]⁺

### EXAMPLE 16

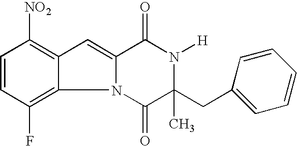

At 0° C., sodium hydride (0.26 g, 2.5 equivalents) was added to a mixture of 3-benzyl-6-(2,3-difluoro-6-nitrobenzylidene)piperazine-2,5-dione (1 g; prepared analogously to Example 1.5) in DMF (20 ml). The mixture was stirred at 0° C. for 1 h, and methyl iodide (1.8 g) was then added. The reaction mixture was stirred at 0° C. for 1 h and at room temperature for 1 h and then quenched with water (50 ml). The aqueous reaction mixture was extracted repeatedly with tent-butyl methyl ether. The resulting organic phase was washed, dried over sodium sulfate, filtered and concentrated. This gave 0.35 g of the title compound after purification by column chromatography.

RT HPLC/MS: 3.403 min., 382.1 [M+H]⁺

### EXAMPLE 17

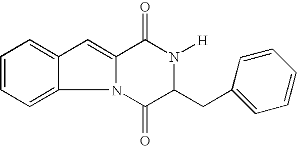

CuCN (0.54 g) was added to 3-benzyl-6-(2-bromobenzylidene)piperazine-2,5-dione from Example 1.5 (1.11 g) in NMP (10 ml). The reaction mixture was stirred at 140° C. for 14 h and then allowed to cool. The reaction mixture was then introduced into 250 ml of water. The mixture was extracted five times with CH₂Cl₂. The organic phase obtained in this manner was washed twice with water, dried over Na₂SO⁴, filtered and concentrated. This gave a brown oil (0.6 g) which solidified on standing. Melting point: 167° C.

RT HPLC/MS: 2.903 min., 290.9 [M+H]⁺

### EXAMPLE 18

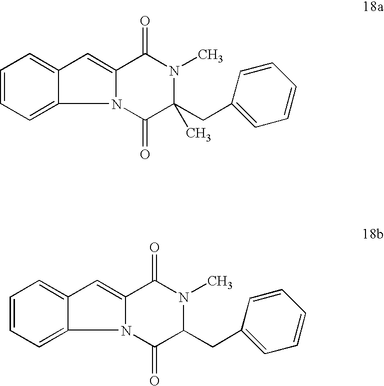

Under argon and at −15° C., sodium hydride (80 mg, 60% pure) was added to the compound from Example 17 (0.32 g) in 5 ml of DMF, the mixture was stirred at −15° C. for 3 h and methyl iodide (1.42 g) was then added. The reaction mixture was stirred at −15° C. for 3 h and at room temperature for 18 h and then introduced into a solution of citric acid (1 g) in water (50 ml). The aqueous reaction mixture was extracted four times with dichloromethane. The organic phase obtained was washed twice with water, dried over sodium sulfate, filtered and concentrated. Purification by flash chromatography (methyl tert-butyl ether) gave the title compound 9a (130 mg) and the title compound 9b (40 mg).

Compound 18a:

RT HPLC/MS: 3.339 min., 319.4 [M+H]⁺

Compound 18b:

RT HPLC/MS: 3.088 min., 304.9 [M+H]⁺

### EXAMPLE 19

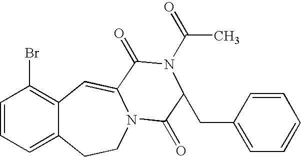

K₂CO₃ (9.1 g) was added to a mixture of 2-bromo-6-vinylbenzaldehyde (9.9 g) and 1,4-diacetyl-3-benzylpiperazine-2,5-dione (14.0 g) from Example 1.3 in 100 ml of DMF. The reaction mixture was stirred at room temperature for 12 h, water was then added and the mixture was extracted repeatedly with CH₂Cl₂. The organic phases obtained in this manner were combined, washed with water, dried over Na₂SO₄ and filtered, and the solvent was removed under reduced pressure. Purification by column chromatography (ethyl acetate: hexane 1:5 1:1) gave 15.7 g of the highly impure title compound.

### EXAMPLE 20

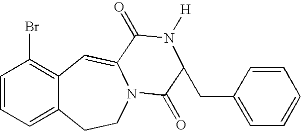

Dilute hydrochloric acid (5% strength, 150 ml) was added to a mixture of 15.7 g of the compound from Example 19 in 100 ml of THF. The reaction mixture was heated under reflux for 8 h and then filtered. The filtrate gave a first precipitate which was washed with water and THF. This gave 8.1 g of the title compound.

RT HPLC/MS: 3.032 min., 399.0 [M+H]⁺

### EXAMPLE 21

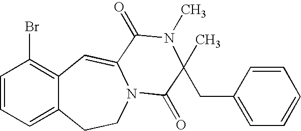

At 0° C., NaH (60% pure, 0.02 mol) was added to the compound from Example 20 (0.01 mol) in DMF (50 ml. The mixture was stirred at 0° C. for 1 h, and methyl iodide (0.1 mol) was then added. The reaction mixture was stirred at room temperature for 18 h and then introduced into a solution of water (500 ml) and citric acid (5 g). The mixture was extracted repeatedly with CH₂Cl₂. The organic phase obtained in this manner was washed with water, dried over Na₂SO₄, filtered and concentrated. Trituration with diisopropyl ether gave the title compound.

RT HPLC/MS: 3.447 min., 426.0 [M+H]⁺

### EXAMPLE 22

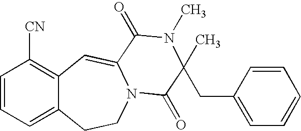

0.63 g CuCN (0.007 mol) was added to the compound from Example 21 (1 g) in 20 ml of NMP. The mixture was stirred at 150° C. for 18 h and then introduced into a solution of water (50 ml) and NaCN (3 g). The mixture was extracted repeatedly with ethyl acetate. The organic phase obtained in this manner was washed with water, dried over Na₂SO₄, filtered and concentrated. After purification by column chromatography and trituration with diisopropyl ether, the desired product was obtained as a solid (0.45 g) of melting point 136-138° C.

RT HPLC/MS: 3.093 min., 372.1 [M+H]⁺

The preparation of the compounds from Examples 23 to 25 was carried out analogously to Examples 15 to 22 shown above.

### EXAMPLE 23

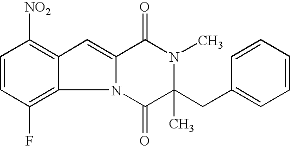

mp.: 170-172° C.; RT HPLC/MS: 3.403 min; m/z=382.1 [M+H]

### EXAMPLE 24

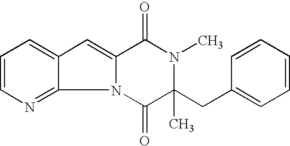

mp.: 180° C.; RT HPLC/MS: 2.514 min; m/z=320.0 [M+H]⁺;

### EXAMPLE 25

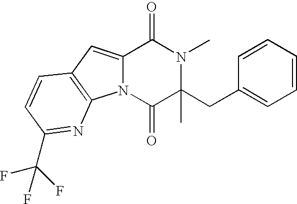

RT HPLC/MS: 3.191 min; m/z=388.1; [M+H]⁺

### EXAMPLE 26

### 3-Benzyl-6-(7-fluoro-3-oxo-2,3-dihydro-1H-isoindol-1-yl)-1,3,4-trimethyl-piperazine-2,5-dione

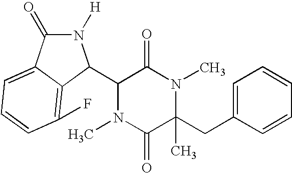

CuCN (8.1 g) was added to 3-benzyl-6-(2-fluoro-6-bromobenzylidene)-1,4,5-trimethylpiperazine-2,5-dione (19.4 g) (prepared analogously to Example 1.6 with an excess of sodium hydride and methyl iodide) in 50 ml NM. The mixture was stirred at 150° C. for 18 h and then introduced into a solution of water (50 ml) and NaCN (3 g). The mixture was extracted repeatedly with ethyl acetate. The organic phase obtained in this manner was washed with water, dried over Na₂SO₄, filtered and concentrated. After purification by column chromatography and trituration with diisopropyl ether, the desired product was obtained as a solid of melting point 183-185° C.

RT HPLC/MS: 2.343 min., 396.1 [M+H]⁺

### EXAMPLE 27

### 3-Benzyl-1,4-dimethyl-6-(3-oxo-1,3-dihydroisobenzofuran-1-yl)-piperazine-2,5-dione

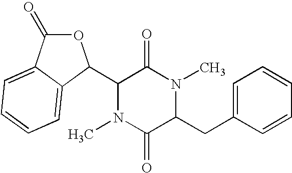

At −78° C., lilthium diisopropylamide (LDA) in THF (20 ml, 2 molar in THF, 0.04 mol) was added to a mixture of 1,4-dimethyl-3-benzylpiperazine-2,5-dione (9.30 g, 0.04 mol, from Example 1.3) in 200 ml of absolute THF. The mixture was stirred at −78° C. for 5 h, and a solution of methyl 2-formylbenzoate (13 g, 0.08 mol) in THF (50 ml) was then added over a period of 30 min. The reaction mixture was stirred at −78° C. for 2 h and at room temperature for 18 h. The mixture was acidified with 1% strength hydrochloric acid solution and concentrated, and the residue was taken up in ethyl acetate. The mixtures was washed four times with sodium bicarbonate solution, and the organic phase was dried over sodium sulfate and concentrated. The residue obtained was triturated with a small amount of acetone, which gave a white solid (0.35 g) of melting point 225° C.

RT HPLC/MS: 2.448 min., 365.1 [M+H]⁺

### EXAMPLE 28

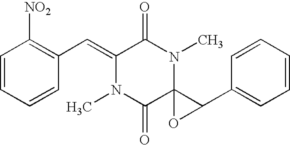

A mixture of 3-(bromo-phenyl-methyl)-3-hydroxy-1,4-dimethyl-6-[1-(2-nitrophenyl)-meth-(Z)-ylidene]piperazine-2,5-dione from Example 14 (0.190 g, 0.0004 mol), triethylamine (0.5 ml) and ethylacetate (20 ml) was refluxed for 4 h. The reaction mixture was cooled, washed with water, dried over Na₂SO₄, filtered and freed from solvent under reduced pressure. Purification by column chromatography (silica gel, hexane/ethyl acetate, 1:1) gave the title compound in an amount of 0.023 g (yield 15%).

RT HPLC-MS: 2.466 min; m/z=398.1 [M+H₂O]⁺.

### EXAMPLE 29

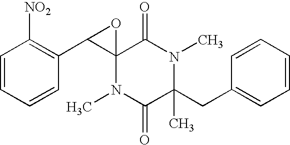

The preparation of the title compound was carried out analogously to Example 28.

RT HPLC/MS: 2.637 min; m/z=395.9 [M+H]⁺.

### EXAMPLE 30

### 6-[1-(2-Nitrophenyl)-meth-(Z)-ylidene]-4,7-dimethyl-1-phenyl-4,7-diazaspiro[2.5]octane-5,8-dione

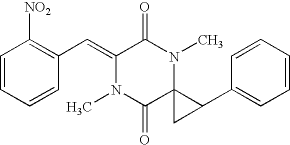

The preparation of the title compound was carried out analogously to steps 1.1 to 1.6 in Example 1, starting from 1-amino-2-phenylcyclopropanecarboxylic acid (prepared as described from Davies, Huw M. L.; McAfee, Melinda J.; Oldenburg, Claes E. M. (1989) Journal of Organic Chemistry 54, 930-936).

mp.: 145° C.; RT HPLC/MS 3.161 min, m/z=378.1 [M+H]⁺

The preparation of the compounds from Example 31 to 34 was carried out in analogy to Example 30.

### EXAMPLE 31

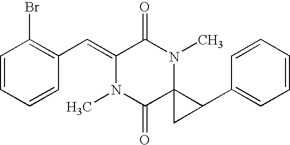

Z-Isomer; mp. 78° C.; RT HPLC/MS: 3.555 min; m/z=413.2 [M+H]⁺

### EXAMPLE 32

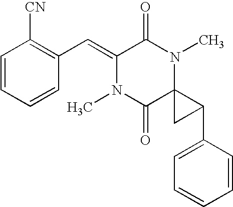

2 Isomers were obtained, in which the exo double bond at the piperazine ring has the (Z) configuration in each case.

(Z)-Isomer 1: mp.: 190° C.; RT HPLC/MS: 3.235 min; m/z=358.2 [M+H]⁺;

(Z)-Isomer 2: mp.: 184° C.; RT HPLC/MS: 3.175 min, m/z=3582 [M+H]⁺

### EXAMPLE 33

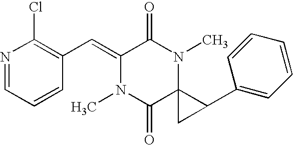

Z-Isomer;

mp.: 169° C.; RT HPLC/MS: 2.826 min, m/z=368.0 [M+H]⁺

### EXAMPLE 34

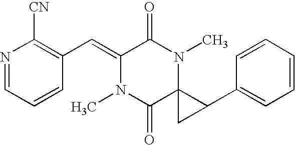

2 Isomers were obtained, in which the exo double bond at the piperazine ring has the (Z) configuration in each case.

(Z)-Isomer 1: mp.: 185° C.; RT HPLC/MS: 2.755 min, m/z=359.1 [M+H]⁺

(Z)-Isomer 2: mp.: 224° C.; RT HPLC/MS 2.827 min m/z=381.1 [M+Na]⁺

### EXAMPLE 35

### 2-[5-Benzyl-1,4,5-trimethyl-3,6-dithioxopiperazin-(2Z)-ylidenemethyl]-benzonitrile

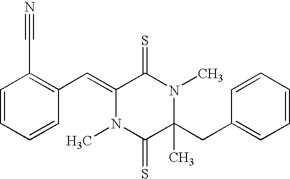

A mixture of 2-[5-benzyl-1,4-dimethyl-3,6-dioxopiperazin-(2Z)-ylidenemethyl]-benzonitrile from Example 1.7 (1.4 g, 3.8 mmol) and phosphorous pentasulfide (5.12 g, 11.5 mmol) in toluene (40 ml) were refluxed for 3.5 h. The reaction mixture was diluted with ethyl acetate (50 ml) and filtered through silica gel. The filtrate was freed from solvent under reduced pressure and purified by column chromatography (silica gel, hexane/ethyl acetate). 505 mg (yield: 34%) of the title compound were obtained as isomer mixture (2 isomers).

RT HPLC-MS: 3.872 min and 3.995 min, [m/z]: 391.8 [M+H]⁺

HPLC-column: RP-18 column (XTerra MS 5 mm from Waters) Eluent: acetonitrile+0.1% formic acid (A)/water+0.1% formic acid in (B). Gradient: from 5:95 (A/B) to 100:0 (A/B) in 8 minutes, at room temperature.

MS: Quadrupol Electrospray Ionisation, 80 V (positive mode)

### EXAMPLE 36

### 2-[5-Benzyl-3,6-bis[ethoxyimin]-1,4,5-trimethylpiperazin-(2Z)-ylidenemethyl]-benzonitrile

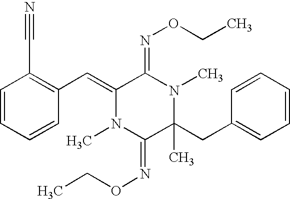

A mixture of 2-[5-benzyl-1,4,5-trimethyl-3,6-dithioxopiperazin-(2Z)-ylidenemethyl]-benzonitrile from Example 35 (0.21 g, 0.5 mmol), O-ethylhydroxylamine hydrochloride (0.17 g, 1.7 mmol), mercury(II) acetate (0.38 g, 1.2 mmol) and diisopropylethylamine (0.9 g, 7.0 mmol) in acetonitrile (15 ml) was stirred at room temperature for 24 h. O-ethylhydroxylamine hydrochloride (0.17 g, 1.7 mmol), mercury(II) acetate (0.38 g, 1.2 mmol) and diisopropylethylamine (0.9 g, 7.0 mmol) were again added and the reaction mixture was stirred for further 24 hours. Then, trichloromethane (20 ml) and a saturated aqueous solution of NH₄Cl (20 ml) were added. The mixture was filtered and the water phase was repeatedly extracted with trichloromethane. The organic phases were combined, washed with water, dried over Na₂SO₄, filtered and freed from solvent under reduced pressure. After purification by column chromatography (silica gel, hexane/ethyl acetate), 147 mg (yield: 61%) of the title compound were obtained as isomer mixture (3 isomers).

HPLC-MS RT: 8.483 min, 8.271 min, 8.030 min; [m/z]: 446.2 [M+H]⁺

HPLC-column: RP-18 column (XTerra MS 5 mm from Waters) Eluent:acetonitrile+0.1% formic acid (A)/water+0.1% formic acid in (B). Gradient: from 5:95 (A/B) to 100:0 (A/B) in 8 minutes, at room temperature.

MS: Quadrupol Electrospray Ionisation, 80 V (positive mode)

### EXAMPLE 37

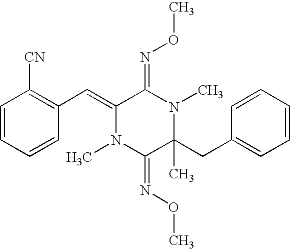

The preparation of the title compound was carried out analogously to Example 36. 2 isomers; RT HPLC MS: 7.617 min and 7.335 min; m/z=418.2 [M+H]⁺

HPLC-column: RP-18 column (XTerra MS 5 mm from Waters) Eluent:acetonitrile+0.1% formic acid (A)/water+0.1% formic acid in (B). Gradient: from 5:95 (A/B) to 100:0 (A/B) in 8 minutes, at room temperature.

MS: Quadrupol Electrospray Ionisation, 80 V (positive mode)

### EXAMPLE 38

### 6-Benzyl-1-(2-bromophenyl)-4,7-dimethyl-4,7-diazaspiro[2.5]octane-5,8-dione

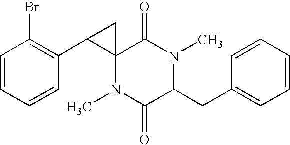

### 38.1 2-(2-Bromophenyl)-1-nitrocyclopropane carboxylic acid methyl ester

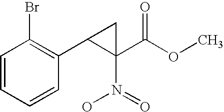

Diazonitroacetic acid methyl ester (prepared as described in O'Bannon, P. E.; Dailey, W. P., Tetrahedron, 1990, 46(21), 7341-7358) (11.9 g, 0.82 mol) was slowly added to a mixture of 2-bromostyrene (15 g, 0.82 mol) and dirhodium(II) tetraacetate (0.5 g, 0.001 mol) in CH₂Cl₂ (500 ml) at room temperature. The reaction mixture was stirred at room temperature for 1 h and freed then from solvent under reduced pressure. After purification by column chromatography (silica gel, hexane/ethyl acetate, 20:1), 2-(2-bromophenyl)-1-nitrocyclopropane carbxylic acid methyl ester was obtained as an oil in an amount of 11.3 g (yield 46%). HPLC-MS [m/z]: 301.1 [M+H]⁺

### 38.2 1-Amino-2-(2-bromophenyl)-cyclopropane carboxylic acid methyl ester

Dilute hydrochloric acid (5% strength, 450 ml) and zinc powder (77 g, 1.18 mol) were added in portions to a mixture of 2-(2-bromophenyl)-1-nitrocyclopropane carboxylic acid methyl ester (17.6 g, 0.59 mol) in isopropanol (450 ml) at room temperature. The reaction mixture was stirred at room temperature for 30 min and then a saturated aqueous solution of sodium hydrogen carbonate was added for neutralization. The precipitated solids were sucked off through silica gel and washed repeatedly with ethyl acetate. The organic phases were combined, washed with water, dried over Na₂SO₄, filtered and freed from solvent under reduced pressure. The isomer mixture obtained was separated by column chromatography (silica gel, hexane/ethyl acetate, 1:1). 3.10 g (yield 20%) of the cis isomer and 7.1 g (yield 45%) of the trans isomer were obtained.

### 38.3 2-(2-Bromophenyl)-1-(1-tert.-butoxycarbonylamino-2-phenyl-ethoxycarbonylamino)-cyclopropanecarboxylic acid methyl ester

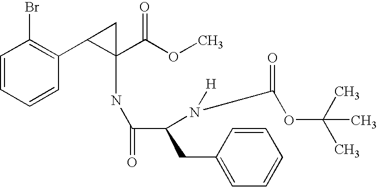

1 Amino-2-(2-bromophenyl)-cyclopropane carboxylic acid methyl ester (3 g, 0.011 mol), BOC-L-phenylalanine (3.15 g, 0.012 mol), O-(7-azabenzotriazole-1-yl)-N,N,N′,N′-tetramethyluronium hexafluorophosphate (HATU) (4.35 g, 0.014 mol) and diisopropylethylamine (4.5 g, 0.035 mol) in CH₂Cl₂ (75 ml) were stirred over night at room temperature and a saturated aqueous solution of citric acid was then added. The mixture was repeatedly extracted ethyl acetate. The organic phases were combined, washed with water, dried over Na₂SO₄, filtered and freed from solvent under reduced pressure. 5.7 g (yield: 99%) of the title compound were obtained as bright oil.

The remaining steps in this synthesis were carried out analogously to Example 1. 4 isomers were aobtained;

RT HPLC MS: 2.936 min; 3.117 min; 2.879 min; 2.874 min; m/z=414.7 [M+H]⁺

### EXAMPLE 39

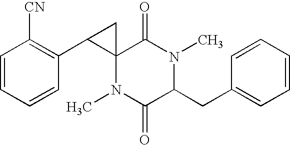

The preparation of the title compound was carried out analogously to Example 1.7, starting from the compound from Example 38.

4 isomers were obtained: RT HPLC MS: 2.500 min; 2.511 min; 2.690 min; 2.689 min; m/z=359.8 [M+H]⁺

Part B: Use Examples

The herbicidal activity of the compounds of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this has been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments belonged to the following species:

| Bayer Code | Scientific name | Common name |
|---|---|---|
| APESV | Apera spica-venti | windgrass |
| SETFA | Setaria faberi | giant foxtail |

The compounds of Examples 1a/1b (ZJE isomer mixture, ZJE ratio=3:1) and 4, applied by the pre-emergence method at an application rate of 0.5 kg/ha, show good to very good herbicidal activity against APESV. The compound of Example 2, applied by the pre-emergence method at an application rate of 1.0 kg/ha, shows very good herbicidal activity against APESV.

The compound of Example 2, applied by the pre-emergence method at an application rate of 1.0 kg/ha, shows good herbicidal activity against SETFA. The compound of Example 4, applied by the pre-emergence method at an application rate of 0.5 kg/ha, shows good herbicidal activity against SETFA.

1-53. (canceled)

54. A piperazine compound of the formula I 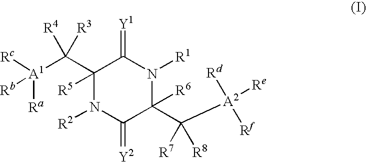
in which
A¹ is phenyl or pyridinyl where R^{a} is attached in the ortho-position to the point of attachment of A¹;
A² is phenyl or thienyl;
Y¹, Y² are oxygen,
R^{a} is selected from the group consisting of halogen, cyano, nitro, SF₅, C₁-C₆-alkyl, C₃-C₆-cycloalkyl, C₂-C₆-alkenyl, C₅-C₈-cycloalkenyl, C₅-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₄-C₁₀-alkadienyl, C₂-C₆-alkynyl, [tri-(C₁-C₆)-alkylsilyl]-(C₂-C₆)-alkynyl, C₇-C₈-cycloalkynyl, aryl, phenyl-(C₁-C₆)-alkyl, phenyl-(C₂-C₆)-alkenyl, phenyl-(C₂-C₆)-alkynyl, phenylsulfonyl-(C₁-C₆)-alkyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, Z¹P(O)(OR⁹)₂, Z¹P(O)(OR⁹)(R^{9a}), Z²B(OR¹⁰)₂, Z³COR¹¹, Z⁴NR¹²R¹³, Z⁵CH═N—O—R¹⁴, Z⁶OR¹⁵, Z⁷SR¹⁶, Z⁷S(O)R¹⁶ and Z⁷SO₂R¹⁶;
where the abovementioned aliphatic, cyclic or aromatic moieties of the substituent R^{a} are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R^{b}, R^{c}, R^{d}, R^{e} and R^{f} are each independently of one another hydrogen or have one of the meanings given for R^{a};
where two radicals R^{a}, R^{b} or R^{c} attached to adjacent ring atoms of A¹ or two radicals R^{d}, R^{e} or R^{f} attached to adjacent ring atoms of A² may also be straight-chain C₃-C₆-alkylene which may be partially or fully halogenated and may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy, in which a CH₂ group in C₃-C₆-alkylene may be replaced by a carbonyl group, thiocarbonyl group or sulfonyl group and in which one or two non-adjacent CH₂ groups in C₃-C₆-alkylene may in each case be replaced by oxygen, sulfur or a group NR³⁴, where R³⁴ has one of the meanings given for R¹²;
R¹ and R² independently of one another are selected from the group consisting of:
cyano, C₁-C₆-alkyl, C₃-C₆-cycloalkyl, C₃-C₆-alkenyl, C₅-C₈-cycloalkenyl, C₃-C⁶-alkynyl, C₇-C₈-cycloalkynyl, C₃-C₆-cycloalkyl-(C₁-C₆)-alkyl, C₅-C₈-cyclo-alkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R²¹, NR²²R²³, OR²⁴, SR²⁴, S(O)R²⁵, SO₂R²⁵ and Si(R^{25a})₃;
where R¹ may additionally be hydrogen and
where the abovementioned aliphatic, cyclic or aromatic moieties of the substituents R¹ and R² independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R³ is halogen, cyano, nitro or a radical R²⁶, OR²⁷, SR²⁸, S(O)R²⁸, SO²R²⁸, NR²⁹R³⁰ or N(OR³¹)R³²;
R⁴ is hydrogen, halogen, cyano, C₁-C₆-alkyl, C₃-C₆-cycloalkyl, C₂-C₆-alkenyl, C₅-C₈-cycloalkenyl, C₂-C₆-alkynyl, C₅-C₈-cycloalkynyl, phenyl, phenyl-(C₁-C₆)-alkyl, heterocyclyl, heterocyclyl-(C₁-C₆)-alkyl, phenyl-[C₁-C₆-alkoxycarbonyl]-C₁-C₆)-alkyl or a radical COR²¹, OR²⁷, SR²⁸, S(O)R²⁸, SO₂R²⁸, NR²⁹R³⁰ or N(OR³¹)R³²,
where the abovementioned aliphatic, cyclic or aromatic moieties of the substituent R⁴ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R⁵ is hydrogen, halogen, cyano, nitro, hydroxyl, C₁-C₈-alkyl, C₂-C₈-alkenyl, C₃-C₈-cycloalkyl, C₅-C₈-cycloalkenyl, C₂-C₈-alkynyl, C₄-C₈-alkadienyl, C₇-C₈-cycloalkynyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, tri-(C₁-C₆)-alkylsilyl, [tri-(C₁-C₆)-alkylsilyl]-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R⁶¹, Z⁸NR⁶²R⁶³, Z¹¹CH═N—O—R⁶⁴, OR⁶⁵, Z⁹SR^{65a}, Z⁹S(O)R⁶⁶, Z⁹S(O)₂R⁶⁶ or Z¹⁰P(O)(OR⁶⁷)₂; or
R³ together with R⁵ is a chemical bond;
R⁶ is halogen, cyano, nitro, C₂-C₈-alkenyl, C₃-C₈-cycloalkyl, C₅-C₈-cycloalkenyl, C₂-C₈-alkynyl, C₄-C₈-alkadienyl, C₇-C₈-cycloalkynyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, tri-(C₁-C₆)-alkylsilyl, [tri-(C₁-C₆)-alkylsilyl]-(C₂-C₆)-alkynyl, phenyl, phenyl-(C₁-C₆)-alkyl, phenyl-(C₂-C₆)-alkenyl, phenyl-(C₂-C₆)-alkynyl, heterocyclyl, heterocyclyl-(C₁-C₆)-alkyl, heterocyclyl-(C₂-C₆)-alkenyl, heterocyclyl-(C₂-C₆)-alkynyl, phenyl-[C₁-C₆-alkoxycarbonyl]-(C₁-C₆)-alkyl, C(O)R⁶¹, Z⁸NR⁶²R⁶³, Z¹¹CH═N—O—R⁶⁴, OR⁶⁵, Z⁹SR^{65a}, Z⁹S(O)R⁶⁶, Z⁹S(O)₂R⁶⁶ or Z¹⁰P(O)(OR⁶⁷)₂;
where the abovementioned aliphatic, cyclic or aromatic moieties of the substituents R⁴, R⁵ and R⁶ independently of one another may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R⁷ is halogen, cyano, nitro or a radical R²⁶, OR²⁷, SR²⁸, S(O)R²⁸, SO₂R²⁸, NR²⁹R³⁰ or N(OR³¹)R³²;
R⁸ has one of the meanings given for R⁴;
R⁹, R¹⁰ and R⁶⁷ are each independently of one another hydrogen or C₁-C₆-alkyl and the radicals R¹⁰ in Z²B(OR¹⁰)₂ may together form a C₂-C₄-alkylene chain; or
R^{9a} is C₁-C₆-alkyl;
R¹¹, R⁶¹ independently of one another are hydrogen, C₁-C₆-alkyl, C₃-C₆-cycloalkyl, C₂-C₆-alkenyl, C₅-C₈-cycloalkenyl, C₂-C₆-alkynyl, C₇-C₈-cycloalkynyl, hydroxyl, C₁-C₆-alkoxy, C₃-C₆-alkenyloxy, C₃-C₆-alkynyloxy, amino, C₁-C₆-alkylamino, di-(C₁-C₆-alkyl)amino, C₁-C₆-alkoxyamino, di-(C₁-C₆-alkoxy)amino, C₁-C₆-alkylsulfonylamino, C₁-C₆-alkylaminosulfonylamino, [di-(C₁-C₆-alkyl)-amino]sulfonylamino, C₃-C₆-alkenylamino, C₃-C₆-alkynylamino, N-(C₂-C₆-alkenyl)-N—(C₁-C₆-alkyl)amino, N—(C₂-C₆-alkynyl)-N—(C₁-C₆-alkyl)-amino, N—(C₁-C₆-alkoxy)-N—(C₁-C₆-alkyl)-amino, N—(C₂-C₆-alkenyl)-N—(C₁-C₆-alkoxy)-amino, N—(C₂-C₆-alkynyl)-N—(C₁-C₆-alkoxy)-amino, phenyl, phenoxy, phenylamino, naphthyl or heterocyclyl;
R¹² and R⁶² independently of one another are hydrogen, C₁-C₆-alkyl, C₁-C₈-alkoxy, C₃-C₆-cycloalkyl, C₃-C₆-cycloalkyloxy, C₃-C₆-alkenyl, C₃-C₆-alkenyloxy, C₅-C₈-cycloalkenyl, C₃-C₆-alkynyl, C₃-C₆-alkynyloxy, C₇-C₈-cycloalkynyl, C₁-C₆-alkylcarbonyl, C₃-C₆-cycloalkylcarbonyl, di-(C₁-C₆-alkyl)aminocarbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, di-(C₁-C₆-alkyl)aminosulfonyl, phenylcarbonyl, phenylaminocarbonyl, phenylsulfonyl, phenylsulfonylaminocarbonyl or heterocyclylcarbonyl;
R¹³ and R⁶³ independently of one another are hydrogen, C₁-C₆-alkyl, C₃-C₆-cycloalkyl, C₃-C₆-alkenyl, C₅-C₈-cycloalkenyl, C₃-C₆-alkynyl, C₃-C₆-alkynyloxy, C₇-C₈-cycloalkynyl, C₁-C₆-alkylcarbonyl, C₃-C₆-cycloalkylcarbonyl, di-(C₁-C₆-alkyl)aminocarbonyl, C₁-C₆-alkoxycarbonyl, C₁-C₆-alkoxycarbonyl-(C₁-C₆)-alkyl, C₁-C₆-alkylsulfonyl, C₁-C₆-alkylaminosulfonyl, di-(C₁-C₆-alkyl)aminosulfonyl, phenylcarbonyl, phenylaminocarbonyl, phenylsulfonyl, phenylsulfonylaminocarbonyl or heterocyclylcarbonyl;
R¹⁴, R⁶⁴ independently of one another are hydrogen, C₁-C₆-alkyl, C₂-C₆-alkenyl or phenyl;
R¹⁵, R^{65a} independently of one another are hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl, phenyl-(C₁-C₆)-alkyl, hetaryl or hetaryl-(C₁-C₆)-alkyl;
R⁶⁵ is C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, C₁-C₆-alkylcarbonyl, C₁-C₆-alkoxycarbonyl-(C₁-C₆)-alkyl, [di-(C₁-C₆)-alkoxycarbonyl]-(C₁-C₆)-alkyl, phenyl or phenyl-(C₁-C₆)-alkyl;
R¹⁶, R⁶⁶ independently of one another are C₁-C₆-alkyl, C₁-C₆-alkoxy, phenyl or phenoxy;
Z¹, Z², Z³, Z⁴, Z⁵, Z⁶, Z⁷, Z⁸, Z⁹, Z¹⁰ and Z¹¹ independently of one another are a bond, —CH₂—, —CH₂—CH₂—, —O—CH(R¹⁷)—, —S—CH(R¹⁸)—, —S(O)—CH(R¹⁹)— or —SO₂CH(R²⁰—, in which R¹⁷, R¹⁸, R¹⁹ and R²⁰ independently of one another are hydrogen or C₁-C₆-alkyl;
R²¹ is hydrogen, C₁-C₆-alkyl, C₃-C₆-cycloalkyl, C₂-C₆-alkenyl, C₅-C₈-cycloalkenyl, C₂-C₆-alkynyl, C₇-C₈-cycloalkynyl, hydroxyl, C₁-C₆-alkoxy, C₃-C₆-alkenyloxy, C₃-C₆-alkynyloxy, amino, C₁-C₆-alkylamino, di-(C₁-C₆-alkyl)amino, C₃-C₆-alkenylamino, C₃-C₆-alkynylamino, C₁-C₆-alkylsulfonylamino, N—(C₂-C₆-alkenyl)-N—(C₁-C₆-alkyl)amino, N—(C₂-C₆-alkynyl)-N—(C₁-C₆-alkyl)-amino, N—(C₁-C₆-alkoxy)-N—(C₁-C₆-alkyl)-amino, phenyl, phenylamino, phenoxy, naphthyl or heterocyclyl;
R²² and R²³ independently of one another are hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl or C₁-C₆-alkylcarbonyl;
R²⁴ is hydrogen, C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, C₃-C₆-cycloalkyl-(C₁-C₆)-alkyl, C₅-C₈-cycloalkenyl-(C₁-C₆)-alkyl, C₅-C₈-cycloalkynyl-(C₁-C₆)-alkyl, C₃-C₆-cycloalkyl-(C₂-C₆)-alkenyl, C₅-C₈-cycloalkenyl-(C₂-C₆)-alkenyl, C₅-C₈-cycloalkynyl-(C₂-C₆)-alkenyl, C₃-C₆-cycloalkyl-(C₂-C₆)-alkynyl, C₅-C₈-cycloalkenyl-(C₂-C₆)-alkynyl, C₅-C₈-cycloalkynyl-(C₂-C₆)-alkynyl, phenyl or phenyl-(C₁-C₆)-alkyl;
R²⁵ is C₁-C₆-alkyl, C₁-C₆-alkoxy, phenyl or phenoxy;
R²⁵′ is C₁-C₆-alkyl, C₃-C₆-cycloalkyl, C₃-C₆-alkenyl, C₅-C₈-cycloalkenyl, C₃-C₆-alkynyl, C₇-C₈-cycloalkynyl, phenyl or phenyl-(C₁-C₆)-alkyl;
where the abovementioned aliphatic, cyclic or aromatic moieties of the substituents R⁹, R^{9a}, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R^{25a}, R⁶¹, R⁶², R^{62a}, R⁶³, R⁶⁴, R⁶⁵, R^{65a}, R⁶⁶ and R⁶⁷ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy;
R²⁶, R²⁷, R²⁸, R²⁹ and R³² independently of one another are hydrogen, C₁-C₆-alkyl, C₃-C₆-cycloalkyl, C₃-C₆-alkenyl, C₃-C₆-alkynyl, formyl, C₁-C₆-alkylcarbonyl, C₃-C₆-cycloalkylcarbonyl, C₂-C₆-alkenylcarbonyl, C₂-C₆-alkynylcarbonyl, C₁-C₆-alkoxy-(C₁-C₆)-alkyl, C₁-C₆-alkoxycarbonyl, C₂-C₆-alkenyloxycarbonyl, C₃-C₆-alkynyloxycarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, C₁-C₆-alkylaminocarbonyl, di-(C₁-C₆-alkyl)aminocarbonyl, N—(C₃-C₆-alkenyl)-N—(C₁-C₆-alkyl)-aminocarbonyl, N—(C₃-C₆-alkynyl)-N—(C₁-C₆-alkyl)-aminocarbonyl, N—(C₁-C₆-alkoxy)-N—(C₁-C₆-alkyl)-aminocarbonyl, N—(C₃-C₆-alkenyl)-N—(C₁-C₆-alkoxy)-aminocarbonyl, N—(C₃-C₆-alkynyl)-N—(C₁-C₆-alkoxy)-aminocarbonyl, di-(C₁-C₆-alkyl)-aminothiocarbonyl, C₁-C₆-alkylcarbonyl-C₁-C₆-alkyl, C₁-C₆-alkoxyimino-C₁-C₆-alkyl, N—(C₁-C₆-alkylamino)-imino-C₁-C₆-alkyl, N-(di-C₁-C₆-alkylamino)-imino-C₁-C₆-alkyl or [tri-(C₁-C₄)-alkyl]silyl, where the abovementioned aliphatic or isocyclic moieties of the substituents may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl, C₁-C₄-alkylcarbonyloxy, phenyl, phenyl-C₁-C₆-alkyl, phenylcarbonyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)-aminocarbonyl, phenyl-C₁-C₆-alkylcarbonyl, heterocyclyl, heterocyclyl-C₁-C₆-alkyl, heterocyclylcarbonyl, heterocyclylcarbonyl-C₁-C₆-alkyl, heterocyclyloxycarbonyl, heterocyclylaminocarbonyl, heterocyclylsulfonylaminocarbonyl, N—(C₁-C₆-alkyl)-N-(heterocyclyl)-aminocarbonyl, or heterocyclyl-C₁-C₆-alkylcarbonyl, where the phenyl or heterocyclyl moieties of the substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or S(O)OR³³, where n is 1 or 2;
R³⁰ and R³¹ independently of one another are hydrogen; C₁-C₆-alkyl; C₃-C₆-cycloalkyl; C₂-C₆-alkenyl; C₂-C₆-alkynyl, where aliphatic or isocyclic moieties of the substituents R³⁰ and R³¹ independently of one another are unsubstituted or may be partially or fully halogenated and/or may carry one to three of the following groups: cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)-amino, C₁-C₄-alkylcarbonyl, hydroxycarbonyl, C₁-C₄-alkoxycarbonyl, aminocarbonyl, C₁-C₄-alkylaminocarbonyl, di-(C₁-C₄-alkyl)aminocarbonyl or C₁-C₄-alkylcarbonyloxy; phenyl; phenyl-C₁-C₆-alkyl; heterocyclyl; or heterocyclyl-C₁-C₆-alkyl, where the phenyl or heterocyclyl moieties of the substituents may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; and
R³³ is C₁-C₆-alkyl, C₁-C₆-haloalkyl or phenyl, where the phenyl substituent may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy;
where one or more of the following provisions may also be met:
a) R² together with one of the radicals R^{a} or R^{b} is a covalent bond or a 1-, 2-, 3- or 4-membered carbon chain in which one carbon atom may be replaced by O, S or a group NR^{F}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
b) R³ together with the radical R⁵ is a 1-, 2-, 3- or 4-membered carbon chain in which one carbon atom may be replaced by O, S or a group NR^{I}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy;
where R⁶ is not C₁-C₆-alkoxy and may be partially or fully halogenated and/or may carry one to three of the following groups:
cyano, hydroxyl, C₁-C₄-alkyl, C₁-C₄-haloalkyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, [di-(C₁-C₄-alkyl)amino]carbonyl or C₁-C₄-alkylcarbonyloxy, if R³ together with R⁵ is a chemical bond;
or a salt thereof.

55. The compound according to claim 54 in which A¹ and A² are each phenyl.

56. The compound according to claim 54 in which:
R^{a} is selected from the group consisting of halogen, cyano, nitro, C(═O)—R¹¹, phenyl and a 5- or 6-membered heterocyclic radical which has 1, 2, 3 or 4 heteroatoms selected from the group consisting of O, N and S as ring atoms, where phenyl and the heterocyclic radical are unsubstituted or may have 1, 2, 3 or 4 substituents independently of one another selected from the group consisting of halogen, CN, NO₂, C₁-C₄-alkyl, C₁-C₄-haloalkyl, C₁-C₄-alkoxy and C₁-C₄-haloalkoxy, where
R¹¹ is hydrogen, C₁-C₆-alkyl, hydroxyl, C₁-C₆-alkoxy, C₃-C₆-alkenyloxy, C₃-C₆-alkynyloxy, amino, C₁-C₆-alkylamino, [di-(C₁-C₆)-alkyl]amino, C₁-C₆-alkoxy-amino, N—C₁-C₆-alkoxy-N—C₁-C₆-alkylamino, C₁-C₆-alkylsulfonylamino, C₁-C₆-alkylaminosulfonylamino, [di-(C₁-C₆)-alkylamino]sulfonylamino, phenyl, phenoxy, phenylamino, naphthyl or heterocyclyl, and the abovementioned aliphatic, cyclic or aromatic moieties of the substituent R¹¹ may be partially or fully halogenated.

57. The compound of claim 54 in which
R^{b} is hydrogen, halogen, nitro, cyano, C₁-C₄-alkyl, C₁-C₄-haloalkyl, C₂-C₄-alkenyl, C₁-C₄-alkoxy, C₁-C₄-haloalkoxy, benzyl or a group S(O)ₙR^{16a} in which R^{16a} is C₁-C₄-alkyl or C₁-C₄-haloalkyl and n is 0, 1 or 2; and
R^{c} is hydrogen or halogen.

58. The compound of claim 54 in which
R^{d}, R^{e} independently of one another are selected from the group consisting of hydrogen, halogen, CN, NO₂, C₁-C₄-alkyl, C₁-C₄-haloalkyl, C₂-C₄-alkenyl, C₁-C₄-alkoxy and C₁-C₄-haloalkoxy; and
R^{f} is hydrogen.

59. The compound of claim 54 in which
R¹ is hydrogen, C₁-C₆-alkyl or C₁-C₆-alkylcarbonyl.

60. The compound of claim 54 in which
R² is C₁-C₆-alkyl or C₁-C₆-alkylcarbonyl.

61. The compound of claim 54 in which
R³ is R²⁶ or OR²⁷, where
R²⁶ and R²⁷ independently of one another are selected from the group consisting of hydrogen, C₁-C₆-alkyl C₁-C₆-alkylcarbonyl, phenyl-C₁-C₆-alkyl, phenylcarbonyl, where the abovementioned aliphatic or aromatic moieties of the substituents may be partially or fully halogenated, and a group SO₂R³³, where
R³³ is C₁-C₆-alkyl or phenyl, and where the phenyl substituent may be partially or fully halogenated and/or may carry one to three C₁-C₆-alkyl groups.

62. The compound according to claim 61 in which R³ is hydrogen.

63. The compound of claim 54 in which R⁴ is hydrogen.

64. The compound of claim 54 in which R⁵ is hydrogen, methyl or hydroxyl.

65. The compound of claim 54 in which R³ together with R⁵ is a chemical bond.

66. The compound of claim 54 in which
R⁶ is halogen, cyano, nitro, C₂-C₈-alkenyl, C₂-C₈-alkynyl or C(O)R⁶¹.

67. The compound of claim 54 in which R⁷ and R⁸ are hydrogen.

68. The compound of claim 54 in which R¹ together with a radical R⁶ is a 3-, 4- or 5-membered carbon chain in which one carbon atom may be replaced by O, S or a group NR^{D}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy.

69. The compound of claim 68 in which R¹ together with the radical R⁶ is CH₂CH₂CH₂ or CH₂CH₂CH₂CH₂ in which 1, 2, 3 or 4 of the hydrogen atoms may be replaced by radicals selected from the group consisting of halogen, cyano, hydroxyl, C₁-C₄-alkyl, C₁-C₄-haloalkyl, C₁-C₄-alkoxy and C₁-C₄-haloalkoxy.

70. The compound of claim 54 in which R³ together with the radical R⁵ is a 1-, 2-, 3- or 4-membered carbon chain in which one carbon atom may be replaced by O, S or a group NR^{I}, where one of the carbon atoms may carry a carbonyl oxygen atom and/or in which the carbon atoms, in addition to hydrogen, may carry 1, 2, 3 or 4 radicals selected from the group consisting of halogen, cyano, hydroxyl, C₁-C₄-alkyl, C₁-C₄-haloalkyl, C₁-C₄-alkoxy and C₁-C₄-haloalkoxy.

71. The compound of claim 70 in which R³ together with the radical R⁵ is CH₂, O or a group NR^{I} in which R^{I} is hydrogen or C₁-C₄-alkyl.

72. A composition comprising a herbicidally effective amount of the piperazine compound of claim 54 or an agriculturally useful salt thereof and auxiliaries customary for formulating crop protection agents.

73. A method for controlling unwanted vegetation wherein a herbicidally effective amount of the piperazine compound of claim 54 or an agriculturally useful salt thereof is applied to on plants, their seed and/or their habitat.
